SEQUENCE LISTING (11 sequences)

SEQ ID NO: 1 - DNA, 44 bp, Homo sapiens
  source 1..44: mol_type = genomic DNA
cgaaggacaacataaagcacgtcccgggaggcggcagcgtgagt

SEQ ID NO: 2 - DNA, 44 bp, synthetic construct
  source 1..44: mol_type = other DNA
cgaaggacaacataaagcacgtctcgggaggcggcagcgtgagt

SEQ ID NO: 3 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - RNA, 21 nt, synthetic construct
  source 1..21: mol_type = other RNA
ggtaagttgctactctttgat

SEQ ID NO: 5 - RNA, 21 nt, synthetic construct
  source 1..21: mol_type = other RNA
ccaatgatggaataggaataa

SEQ ID NO: 6 - RNA, 21 nt, synthetic construct
  source 1..21: mol_type = other RNA
gcaagttcatctacataaccc

SEQ ID NO: 7 - RNA, 19 nt, synthetic construct
  source 1..19: mol_type = other RNA
gcttcgcgccgtagtctta

SEQ ID NO: 8 - RNA, 62 nt, synthetic construct
  source 1..62: mol_type = other RNA
gatccgggtaagttgctactctttgattcaagagatcaaagagtagcaacttaccttttttg

SEQ ID NO: 9 - RNA, 62 nt, synthetic construct
  source 1..62: mol_type = other RNA
gatccgccaatgatggaataggaataatcaagagttattcctattccatcattggttttttg

SEQ ID NO: 10 - RNA, 62 nt, synthetic construct
  source 1..62: mol_type = other RNA
gatccggcaagttcatctacataaccctcaagaggggttatgtagatgaacttgcttttttg

SEQ ID NO: 11 - RNA, 58 nt, synthetic construct
  source 1..58: mol_type = other RNA
gatccggcttcgcgccgtagtcttatcaagagtaagactacggcgcgaagcttttttg

(19) United States
(12) Patent Application Publication
(10) Pub. No.: US 2024/0409890 A1
(43) Pub. Date: Dec. 12, 2024

(54) UFMYLATION INHIBITION TO TARGET TAUOPATHY IN HUMAN NEURONS

(71) Applicants: Li Gan et al., New York, NY (US); Shiaoching Gong, New York, NY (US); Celeste Parra Bravo, New York, NY (US); Zeping Zhao, Queens, NY (US)

(72) Inventors: Li Gan et al., New York, NY (US); Shiaoching Gong, New York, NY (US); Celeste Parra Bravo, New York, NY (US); Zeping Zhao, Queens, NY (US)

(21) Appl. No.: 18/739,113

(22) Filed: Jun. 10, 2024

Related U.S. Application Data
(60) Provisional application No. 63/507,043, filed on Jun. 8, 2023.
(60) Provisional application No. 63/507,978, filed on Jun. 13, 2023.

(51) Int. Cl. C12N 5/0793; A61K 31/343; A61P 25/28; C07K 14/47; C12N 5/00; C12N 9/22; C12N 15/113; G01N 33/50

(52) U.S. Cl. CPC C12N 5/0619; A61K 31/343; A61P 25/28; C07K 14/47; C12N 5/0018; C12N 9/22; C12N 15/113; G01N 33/5026; G01N 33/5058; C12N 2310/122; C12N 2310/20; C12N 2501/13; C12N 2501/999; C12N 2506/45

(57) ABSTRACT

Provided herein are iPSC lines engineered to express 4R-tau and 4R-tau carrying the P301S MAPT mutation when differentiated into neurons. 4R-P301S neurons display progressive Tau inclusions upon seeding with Tau fibrils and recapitulate features of tauopathy phenotypes, including shared transcriptomic signatures, autophagic body accumulation, and impaired neuronal activity. A CRISPRi screening of genes associated with Tau pathobiology identified over 500 genetic modifiers of Tau-seeding-induced Tau propagation, including retromer VPS29 and the UFMylation cascade as top modifiers. In AD brains, the UFMylation cascade is altered in neurofibrillary-tangle-bearing neurons. Inhibiting the UFMylation cascade suppressed seeding-induced Tau propagation. Also provided herein is a platform to identify novel therapeutic strategies for 4R tauopathy.

### PRIORITY

This application claims the benefit of the filing date of U.S. provisional application No. 63/507,043, filed on Jun. 8, 2023, and U.S. provisional application No. 63/507,978, filed on Jun. 13, 2023, the disclosures of which are incorporated by reference herein in their entirety.

### INCORPORATION BY REFERENCE OF SEQUENCE LISTING

This application contains a Sequence Listing which has been submitted electronically in ST26 format and hereby incorporated by reference in its entirety. Said ST26 file, created on Jul. 10, 2024, is name 1676200US1.xml and is 11,381 bytes in size.

### BACKGROUND OF THE INVENTION

Tauopathies, characterized by accumulation of tau aggregates, are a heterogeneous group of neurodegenerative diseases. They include Alzheimer's disease (AD), the most common tauopathy, and frontotemporal lobar degeneration with Tau pathology (FTLD-Tau), such as corticobasal degeneration (CBD), progressive supranuclear palsy (PSP), argyrophilic grain disease (AGD), globular glial tauopathy, chronic traumatic encephalopathy (CTE) and Pick's disease (PiD) (Gotz et al., 2019). The microtubule-associated protein Tau is encoded by a single gene (MAPT) and gives rise to six isoforms, including isoforms containing either three (3R) or four (4R) microtubule-binding repeats, due to alternative splicing of exon 10 (Goedert et al., 1989). Based on the dominant 3R or 4R isoforms, there are three subtypes of tauopathies, 3R, 4R, and 3R/4R mixed tauopathies that exhibit distinct Tau filament structures revealed by cryogenic electron microscopy (cryo-EM). Tau filaments in AD (3R/4R) (Fitzpatrick et al., 2017), PiD (3R), CBD (4R), and PSP (4R) (Shi et al., 2021) are structurally distinct. Among the MAPT mutations that cause familial cases of FTLD-tau, many alter the ratio of 3R to 4R (Hutton et al., 1998; Spillantini et al., 1998), and several of the mutations, including P301 S/L (Mirra et al., 1999), are located in the exon 10 and therefore 4R-specific.

### SUMMARY

One embodiment provides an induced pluripotent stem cell (iPSC) stably expressing 4R-Tau. In one embodiment, the iPSC is prepared from a fibroblast cell. In one embodiment, the iPSC is a human cell. In one embodiment, the iPSC comprising one or more mutations in the 5′ and or 3′ end of exon 10 of the microtubule associated protein tau (MAPT) gene. One embodiment further comprises inducible expression of Neurogenin-2 transcription factor (NGN2). One embodiment further comprises a nucleic acid mutation in one or both alleles of microtubule associated protein tau (MAPT) so at to result in a mutation at amino acid 301 of MAPT protein. In one embodiment, the amino acid mutation is P301S. In one embodiment, the P301 S mutation occurs in SEQ ID NO: 3 or a polypeptide having 90% identity thereto. One embodiment further comprises a Cas enzyme, such as Cas9.

In one embodiment, provides a composition comprising the iPSCs described herein.

One embodiment provides a method to express 4R-tau, comprising differentiating the iPSC of claim 6 to a neuronal cell. In one embodiment, the iPSC is contacted with one or more of brain-derived neurotrophic (BDNF), neurotrophin-3 (NTS), ROCK inhibitor or doxycycline.

One embodiment provides a method to generate tau bundles/inclusions comprising contacting said neuronal cell of claim 12 with Tau fibrils. In one embodiment, the Tau fibrils have one or more mutations compared to wild type.

One embodiment provides a method to inhibit formation of Tau bundles/inclusions comprising contacting a neuronal cell with an inhibitor of an UFMylation pathway protein. In one embodiment, the UFMylation pathway protein is one or more of UBA5 (E1), UFC1 (E2), UFL1, DDRGK1 and/or CDK5RAP3. In one embodiment, the inhibitor a small molecule, such as Usenamine A. In one embodiment, the inhibitor is an inhibitory nucleic acid sequence, including by not limited to an shRNA, a small interfering RNA, a ribozyme or an antisense nucleic acid molecule. In one embodiment, the nucleic sequence knocks down UBA5, UFM1, UFBP1 or a combination thereof. In one embodiment, the shRNA has the sequence of any one of SEQ ID NOs. 4 to 11.

One embodiment provides a method to treat a tauopathy comprising administering to a subject in need thereof an inhibitor of an UFMylation pathway protein. In one embodiment, the tauopathy is Alzheimer's disease (AD), frontotemporal lobar degeneration with Tau pathology (FTLD-Tau), corticobasal degeneration (CBD), progressive supranuclear palsy (PSP), argyrophilic grain disease (AGD), globular glial tauopathy, chronic traumatic encephalopathy (CTE) or Pick's disease (PiD).

One embodiment provides a method to screen for compounds that inhibit formation of Tau bundles/inclusions comprising: contacting said neuronal cell of claim 12 with Tau fibrils and a test agent; and detecting the presence or absence of Tau bundles/inclusions, wherein the absence of Tau bundles/inclusions correlates with the test agent being a compound that inhibits formation of Tau bundles/inclusions.

### BRIEF DESCRIPTION OF THE DRAWINGS

The drawings illustrate generally, by way of example, but not by way of limitation, various embodiments discussed herein.

FIGS. 1A-1H. Generation and characterization of 4R-Tau human iPSC-derived neurons. (A) Stepwise strategy of CRISPR/Cas9-mediated genome editing to generate MAPT 4R and 4R-P301S knock-in. (B) Representative immunoblot of lysates from 3R (i3N), 4R heterozygous, and 4R homozygous 6-week-old neurons (D42) before and after lambda protein phosphatase treatment. n=2 independent experiments. (C) Representative immunofluorescence images of 6-week-old (D44) i3N and 4R neurons stained with ET3. Scale bar, 100 μm. (D and E) Representative immunoblots (D) and the quantification (E) of phosphorylated tau (AT180, AT270, AT8) and total tau (HT7) in 6-week-old (D42) i3N, 4R homozygous, Tau-KO, and 4R homozygous neurons. Normalized to GAPDH. *p<0.05, ***p<0.001, ****p<0.0001. One-way ANOVA, Tukey's multiple comparison's test. (F and G) Gene set enrichment analysis pathways identified for upregulated (F) and downregulated (G) DEGs in 4R-P301S vs 4R neurons. Significantly enriched Gene Ontology terms for biological process, cellular component, and molecular function are shown. (H) Heatmap and hierarchical clustering of DE genes within the Transmembrane Transport and Intracellular Transport pathways in untreated 4R-P301S and 4R neurons. Upregulated genes >0 (red), Downregulated genes <0 (blue). The genes listed include DE genes based on read count, adjusted p-value <0.05. Gene Ontology term for biological process. Abbreviations: phosphorylated Tau (p-Tau), total Tau (t-Tau), 3R homozygous (i3N), 4R homozygous (4R).

FIGS. 2A-2N. Modeling seeding-induced 4R-Tau inclusions in human neurons. (A) Diagram illustrating the 2-step differentiation of 4R and 4R-P301S human iPSCs into Doxycycline-inducible glutamatergic neurons, followed by K18-P301L Tau fibril (K18) seeding at 1 week, and used for experiments >2 weeks post-seeding. This FIG. was created with Biorender.com. (B) Representative immunofluorescence images of K18-seeded D25(7+18) 4R and 4R-P301S neurons stained with MC1. Scale bar, 25 μm. n=3, three independent experiments. (C and D) Representative images (C) and quantification (D) of immunofluorescence time course quantification of MC1/DAPI+ nuclei in 4R-P301 S neurons 1-5 weeks post-K18 treatment. n=4, two independent experiments performed in replicate. Scale bar, 50 μm. (E) Representative immunofluorescence images of D25(7+18) 4R-P301S neurons seeded with K18 showing the presence of phosphorylated tau (AT8) and oligomeric tau (TTC18). Scale bar, 25 μm. (F) Left panel: Representative TEM image of a 3 μg/ml K18-seeded D35(7+28) 4R-P301S neuronal soma with a large tau inclusion (outlined). Scale bar, 2 μm. Right panel: Enlarged image showing distinct individual tau fibrils resolved upon magnification. Scale bar, 200 nm. (G and H) Representative immunoblot images (G) of detergent-fractionated lysates from D59(D7+52) 4R and 4R-P301S neurons −/+1.5 μg/ml K18 seeding stained with total tau (HT7) and p-Tau (AT8). T=Triton soluble, S=SDS soluble. Quantification (H) of lysates from D68(22+46) and D59(D7+52) 4R and 4R-P301S normalized to GAPDH. n=3, two independent experiments, 1 performed in duplicate. *p<0.05, ***p<0.001, one-way ANOVA, Tukey post-hoc test. (I and J) Representative flow cytometry analysis (I) and quantification (J) of the percentage of MC1+ cells in and D42(7+35) 4R and 4R-P301S neurons clone #1 and clone #2−/+1.5 μg/mL K18 groups. n=3-6 biological replicates. ****p<0.0001, two-way ANOVA, Šídák's multiple comparisons test. (K) Venn diagram comparing the number and overlap of downregulated DEGs in 4R-P301S+K18 vs 4R+K18 neurons from bulk RNA-seq analysis, and AT8+ vs AT8− neurons from pseudo bulk RNA-seq analysis of AD brains. Fisher's exact test. (L) Top gene set enrichment analysis pathways identified for overlapping downregulated DEGs in 4R-P301S+K18 vs 4R+K18/AT8+ vs AT8− comparison. Significantly enriched Gene Ontology terms for biological process, cellular component, and molecular function are shown. (M) Venn diagram comparing the number and overlap of upregulated DEGs in 4R-P301S+K18 vs 4R-P301S neurons from pseudo bulk RNA-seq analysis, and AT8+ vs AT8− neurons from pseudo bulk RNA-seq analysis of AD brains. Fisher's exact test. (N) Top gene set enrichment analysis pathways identified for overlapping upregulated DEGs in 4R-P301S+K18 vs P301S/AT8+ vs AT8− comparison. Significantly enriched Gene Ontology terms for biological process, cellular component, and molecular function are shown. Abbreviations: central nervous system (CNS), transmembrane (TM).

FIGS. 3A-3I. Endolysosomal dysfunction promotes propagation of Tau inclusions. (A) Representative TEM images from soma D35 4R neurons, D40 4R-P301 S neurons, and K18-seeded D40(7+33) 4R-P301S neurons. Red arrow=MLB. Scale bar, 1 μm. (B) Quantification of #MLB/neuron from D35 and D40 4R and 4R-P301S, and D43(7+36) 4R-P301S+K18 neurons from TEM images. n=2 replicates per group, two independent experiments. ***p<0.001, one-way ANOVA, Tukey post-hoc test. (C) Representative TEM images from soma of K18-seeded D43(7+36) 4R-P301S neurons at different magnifications. Scale bar left to right: 2 μm, 500 nm, 200 nm. (D) Representative TEM images form neuronal processes of K18-seeded D35(7+28) 4R and 4R-P301S neurons showing dystrophic neurites presenting with Tau inclusions and increased numbers of MLBs in 4R-P301S neurons. Scale bar left to right: 500 nm, 500 nm, 200 nm, 100 nm. (E) Diagram illustrating VAMP7 dominant-negative (VAMP7DN) functional knockdown mechanism. VAMP7 is truncated by removal of the SNARE motif, preventing interaction with SNARE helix bundle and lysosomal membrane fusion. TM=transmembrane. (F) Schematic illustrating 4R and 4R-P301S iPSC differentiation, transduction with GFP or GFP-VAMP7DN lentiviruses 24-48 hours before 3 μg/ml K18 seeding at D14, and collection of cells for ELISA and staining at D29. (G) Quantification of total Tau secreted from 4R and 4R-P301S neurons −/+3 μg/mL K18 overexpressing GFP or GFP-VAMP7DN incubated with 50 mM KCl for 30 minutes to enhance neuronal activity. Secreted Tau measured by ELISA was normalized to total Tau in the neuron culture. n=2-6 replicates per group, two independent experiments. **p<0.01, ***p<0.001, one-way ANOVA, Tukey's multiple comparisons post hoc test. (H) Representative immunofluorescence images of D28(14+14) K18-seeded 4R-P301S neurons overexpressing GFP or GFP-VAMP7DN. (I) Quantification of MC1 area/DAPI+ nuclei from immunofluorescence images of D28(14+14) K18-seeded 4R-P301S neurons overexpressing GFP or GFP-VAMP7DN. n=3-6 replicates per group, 10-12 Z-stack images each, two independent experiments. ***p<0.001 by unpaired t-test. Scale bar, 50 μm.

FIGS. 4A-4J. Tau inclusions impair spontaneous and evoked neuronal activity. (A) Schematic illustrating the CRISPR-mediated knock-in of HaloTag cassette at 5′ end of MAPT locus in 4R-P301S iPSC line. This FIG. was created with Biorender.com. (B) Representative fluorescence image of 3 μg/ml K18-seeded 4R-P301S-HaloTag neurons (DIV24) harboring tau inclusions (red arrow: −inclusion; green arrow: +inclusion). Incubated with JFX549-HaloTag ligand. Scale bar, 50 μm. (C) Representative fluorescence images expressing jGCaMP8f, HaloTag, and merged two sensors in DIV24 K18-seeded 4R-P301S-HaloTag spontaneous activity. white arrowhead: −inclusion; white arrow: +inclusion. Scale bar, 50 μm. (D) Quantification of spike amplitude, firing rate, and synchrony index from calcium imaging of K18-seeded 4R-P301S-HaloTag neurons (DIV 24-30). n=3 replicates, two independent experiments. *p<0.05 by unpaired t-test. (E) Representative fluorescence images of jGCaMP8f, HaloTag, and merged two sensors in DIV30 18-seeded 4R-P301S-HaloTag 50 mM KCl stimulation-induced activity (I). white arrowhead: −inclusion examples; white arrow: +inclusion examples. Scale bar, 50 μm. (F) Representative averaged calcium traces from one KCl stimulation experiment in 4R-P301S-HaloTag neurons (DIV30) displaying KCl stimulation-induced neuronal responses with (red) and without (black) tau inclusions. Recording 400 s. Mean f SEM. (−inclusion: n=11; +inclusion: n=8). (G) Quantification of peak amplitude from KCl stimulation-induced neuronal responses. n=3 replicates, two independent experiments. *p<0.05 by unpaired t-test. (H) Schematic illustrating 4R-P301 S differentiation, transduction with GCaMP8F and hM4Di DREADD lentiviruses at D6, 3 μg/ml K18 seeding at D7, weekly 10 μM CNO addition starting at D14, and fixing/staining at D29. (I) Representative spontaneous calcium traces (right) of each cell from DIV36 4R-P301S expressing hM4Di (left) indicating acute neuronal silencing upon 10 μM CNO addition at 60 s. (J) Quantification of MC1 area/DAPI+nuclei from immunofluorescence images of chronically silenced D29 K18-seeded 4R-P301S neurons. n=1-3 replicates, 10 Z-stack images each, two independent experiments.

FIGS. 5A-5G. Identification of Tau inclusion modifiers by CRISPRi screening. (A) Strategy for CRISPRi screening based on tau inclusions in 4R-P301S neurons. 4R-P301S iPSCs were transduced with pC13N-dCas9-BFP-KRAB lentivirus to express dCas9, generating 4R-P301S-dCas9 iPSCs. iPSCs were transduced with a custom sgRNA library lentivirus targeting genes involved in tau aggregation and propagation (1073 genes) and differentiated, seeded with K18-tau at D7, and collected at D19. Neurons were stained for MC1 and FACS-sorted to separate MC1+ and MC1− populations. Frequencies of neurons expressing a given sgRNA were determined by next-generation sequencing. (B) Volcano plot summarizing knockdown phenotypes and statistical significance (Mann-Whitney U test) for genes targeted in the pooled screen. MC1+, genes reducing tau inclusions. MC1−, genes enhancing tau inclusions. Dashed lines: cutoff for hit genes (FDR=0.1, see STAR Methods). (C) Subcellular classification of gene hits (product above 2.5 and below −2.5, see STAR methods) highlights roles in the ER/Golgi, recycling and vesicle trafficking machinery, and mitochondria. (D and E) Representative immunofluorescence images (D) and quantification (E) of the percentage of MC1+ cells in D21(7+14) 1.5 μg/ml K18-seeded 4R-P301S;VPS29−/− neurons clone #1 and #2 compared to the parent. n=4 replicates, one independent experiment per clone. ****p<0.0001, one-way ANOVA, Dunnett's multiple comparisons test. Scale bar, 50 μm. (F and G) Representative flow cytometry analysis (F) and quantification (G) of the percentage of MC1+ cells in D21(7+14) 1.5 μg/ml K18-seeded 4R-P301S;VPS29−/− neurons clone #1 and clone #2 compared to the parent. n=6 replicates, one independent experiment per clone. ****p<0.0001, one-way ANOVA, Dunnett's multiple comparisons test.

FIGS. 6A-6L CRISPRi screen identifies the UFMylation pathway as a novel modulator of Tau inclusions. (A) Diagram illustrating the protein components necessary for UFMylation of a target substrate. Abbreviation: VGSC is an amino acid motif (valine-glycine-serine-cysteine). (B and C) Representative flow cytometry analysis (B) and the quantification (C) of the percentage of MC1+ cells in D21(7+14) 3 μg/ml K18-seeded 4R-P301S neurons infected with lentivirus (GFP+) containing shRNAs targeting UFMylation cascade genes (UBA5, UFM1). n=3-6 replicates, two independent experiments, ***p<0.001, ****p<0.0001, mixed model analysis, Dunnett's multiple comparisons test. Scale bar, 50 μm. (D and E) Representative immunofluorescence images (D) and the quantification (E) of the percentage of MC1+ cells in D21(7+14) 3 μg/ml K18-seeded 4R-P301S neurons infected with lentivirus (GFP+) containing shRNAs targeting UFMylation cascade genes (UBA5, UFM1). n=6 replicates, two independent experiments. ****p<0.0001, one-way ANOVA, Dunnett's multiple comparisons test. Scale bar, 50 μm. (F) Representative immunoblot (G) and the quantification (H) of relative free UFM1 D21 (7+14) 3 μg/ml K18-seeded 4R-P30 S-HaloTag detergent-fractionated neuronal samples. Triton soluble (soluble), SDS-soluble (insoluble). Normalized to GAPDH. n=3 replicates per group, two independent experiments. *p<0.05, two-way ANOVA, Šídák's multiple comparisons test. (G) Quantification of relative free UFM1 in SDS-soluble fraction of control vs. K18-seeded neurons. **p<0.01 by unpaired t-test. (H) Representative immunofluorescence images of human AD post-mortem tissue stained with MC1 and UFM1. Arrow with shaft, MC1+ neuron. Arrow without shaft, MC1− neuron. Scale bar, 25 μm. (I) Quantification of UFM1 immunofluorescence normalized fold change intensity in MC1+ and MC1− neurons in human AD post-mortem tissue. n=4 male cases and 3 female cases, 12-15 neurons per group, per case. ****p<0.0001, mixed effects analysis.

FIGS. 7A-7E. Generation of isogenic 4R-tau hiPSCs, related to FIG. 1. (A and B) A normal karyotype was confirmed for the 4R line clones #1 (A) and #2 (B). (C) A normal karyotype was confirmed for 95% of heterozygous 4R cells analyzed (clone #1). (D) Pluripotency marker staining (OCT4, TRA-1-60, NANOG, TRA-1-81, SOX2, SSEA4) confirms 4R (clone #1 and #2) hiPSC pluripotency. Scale bar, 100 μm. (E) Representative immunofluorescence images of 6-week-old (D44) i3N and 4R homozygous clone #2 neurons stained with ET3. Scale bar, 100 μm.

FIGS. 8A-8F. Generation and bulk RNA-sequencing of 4R-P301S hiPSCs, related to FIG. 1. (A) Point mutation (C>T) validation was performed to generate the P301S mutation (SEQ ID NOs: 1 and 2). (B and C) A normal karyotype was confirmed for the 4R-P301S line clones #1 (B) and #2 (C). (D) Pluripotency marker staining (OCT4, TRA-1-60, NANOG, TRA-1-81, SOX2, SSEA4) confirms 4R-P301S (clones #1 and #2) hiPSC pluripotency. Scale bar, 100 μm. (E and F) Principal component analysis (E) and sample-sample correlation (F) of D43 4R and 4R-P301S neurons from bulk RNA-seq. n=5 samples 4R, 5 samples 4R-P301 S.

FIGS. 9A-9I. Modeling 4R-Tau inclusions, related to FIG. 2. (A) Representative immunofluorescence images of D25(7+18) 4R and 4R-P301S neurons −/+K18 stained with MC1. Scale bar, 25 μm. n=3, three independent experiments. (B) Immunoblot of phosphorylated tau (AT270) and total tau (HT7) from detergent-fractionated D68(22+46) 4R, 4R-P301S, and Tau-KO neuronal lysates. (C and D) Representative flow cytometry analysis (C) and the quantification (D) of the percentage of MC1+ cells in D21(7+14) 4R and 4R-P301 S neurons clone #1 and clone #2 −/+1.5 μg/mL K18 groups. n=3-6 biological replicates. ****p<0.0001, two-way ANOVA, Šídák's multiple comparisons test. (E and F) Representative flow cytometry analysis (E) and the quantification (F) of the percentage of MC1+ cells in D21(7+14) 4R and 4R-P301S neurons clone #1 and clone #2−/+3 μg/mL K18 groups. n=3-6 biological replicates. ****p<0.0001, two-way ANOVA, Šídák's multiple comparisons test. (G and H) Representative flow cytometry analysis (G) and the quantification (H) of the percentage of MC1+ cells in D42(7+35) 4R and 4R-P301S neurons clone #1 and clone #2−/+3 μg/mL K18 groups. n=3-6 biological replicates. ****p<0.0001, two-way ANOVA, Šídák's multiple comparisons test. (I) Representative immunofluorescence images of D25(7+18)4R and 4R-P301S neurons −/+0N4R stained with MC1. Scale bar, 25 μm. n=4, three independent experiments.

FIGS. 10A-10G. Bulk and single-cell transcriptomic analyses of 4R tauopathy model, related to FIG. 2. (A and B) Principal component analysis (A) and sample-sample correlation (B) of D43(8+35) 4R and 4R-P301S neurons −/+1.5 μg/ml K18 from bulk RNA-sequencing. n=5 samples 4R+K18, 4 samples 4R-P301S+K18. (C-E) Violin plots showing spread of total genes (C), total UMIs (D), and percent of mitochondrial genes (E) detected per cell for each sample from single-cell RNA-sequencing of D39(7+32) 4R and 4R-P301S neurons. n=1 sample P301S, 1 sample P301S+K18. (F and G) UMAP plot of all single cells (F) and separated by sample (G).

FIGS. 11A-11E. Generation of isogenic 4R-P301S-HaloTag hiPSCs and representative images of gCaMP8f- and hM4Di-transduced neurons, related to FIG. 4. (A and B) A normal karyotype was confirmed for the 4R-P301S-HaloTag line clones #1 (A) and #2 (B). (C) Pluripotency marker staining (OCT4, TRA-1-60, NANOG, TRA-1-81, SOX2, SSEA4) confirms 4R-P301S-HaloTag (clone #1 and #2) hiPSC pluripotency. Scale bar, 100 μm. (D) Representative image of D27 4R and 4R-P301S neurons overexpressing GCaMP8-fast genetically encoded calcium sensor at 488 nm, 30% light climate. Scale bar, 50 μm. (E) Representative fluorescent images of hM4Di-mCherry expression levels in live 4R-P301S-HaloTag neurons before (D12) and after (D14) CNO addition. Scale bar, 100 μm.

FIGS. 12A-12G. Characterization of 4R-P301S-dCas9 lines for CRISPRi screen, related to FIG. 5. (A and B) A normal karyotype was confirmed for the 4R-P301S-dCas9 line clone #1 (A) and clone #2 (B). (C) Pluripotency marker staining (OCT4, TRA-1-60, NANOG, TRA-1-81, SOX2, SSEA4) confirms 4R-P301S-dCas9 (clone #1) hiPSC pluripotency. Scale bar, 50 μm. (D) Representative immunofluorescence images of D21 4R-P301S-dCas9 neurons expressing a TFRC-targeting sgRNA or an NTC sgRNA stained with TFRC antibody. Scale bar, 25 μm. (E) Functional validation of constitutively active CRISPRi activity by flow cytometry of TFRC surface protein level stained D21 4R-P301S-dCas9 neurons expressing a TFRC-targeting sgRNA or an NTC sgRNA. n=3, two independent experiments, ****p<0.0001 by unpaired t test. (F and G) Gene set enrichment analysis results for differentially expressed genes in MC1+ neurons (F) and MC1− neurons (G). Significantly enriched Gene Ontology terms for biological process, cellular component, and molecular function are shown.

FIGS. 13A-13D. Characterization and validation of 4R-P301S;VPS29−/− hiPSCs, related to FIG. 5. (A and B) A normal karyotype was confirmed for the 4R-P301S;VPS29−/− line clone #1 (A) and in 95% of cells analyzed of clone #2 (B). (C) Pluripotency marker staining (OCT4, TRA-1-60, NANOG, TRA-1-81, SOX2, SSEA4) confirms 4R-P301S;VPS29−/− (clone #1, #2) hiPSC pluripotency. Scale bar, 50 μm. (D) Quantification of relative mRNA levels for VPS29 in D21(7+14) K18-seeded 4R-P301S; VPS29−/− neuron clones #1 and #2 compared to parent. n=2 replicates, two independent experiments. ****p<0.0001. One-way ANOVA and Dunnett's multiple comparisons test.

FIGS. 14A-14D. Validation of shRNA targeting UBA5 and UFM1 in 4R-P301 S neurons, related to FIG. 6. (A) Flow cytometry gating strategy for GFP+ neurons. (B-D) Quantification of relative mRNA levels for MAPT (B), UBA5 (C), and UFM1 (D) in D21(7+14) K18-seeded 4R-P301S neurons transduced with lentivirus containing shRNAs targeting UBA5 and UFM1 compared to control shRNA. n=2 replicates, two independent experiments. ***p<0.001. One-way ANOVA and Dunnett's multiple comparisons test (B). Unpaired t-test (C-D).

FIGS. 15A-15B. USE treatment prevented the tau inclusion induced by K18 treatment. (A and B) Representative flow cytometry analysis (A) and the quantification (B) of the percentage of MC1+ cells in D21(7+14) 3 μg/ml K18-seeded 4R-P301S neurons pre-treated with USE at various concentrations. n=4 replicates, one independent experiments, **p<0.01, ns P>0.05, student t test.

Unless otherwise indicated, all FIG.s and drawings in this document are not to scale and are chosen for the purpose of illustrating different embodiments of the invention. The dimensions of the various components are depicted in illustrative terms only, and no relationship between the dimensions of the various components should be inferred from the drawings, unless so indicated. Although terms such as “top”, “bottom”, “upper”, “lower”, “under”, “over”, “front”, “back”, “up” and “down”, and “first” and “second” can be used in this disclosure, it should be understood that those terms are used in their relative sense only unless otherwise noted.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a,” “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

“Homologous” or “identity” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0. and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, an “effective amount” means an amount sufficient to produce a selected effect, such as alleviating symptoms of a disease or disorder. The term to “treat,” as used herein, means reducing the frequency with which symptoms are experienced by a patient or subject or administering an agent or compound to reduce the frequency with which symptoms are experienced.

The term “delivery vehicle” or “carrier” refers to any kind of device or material which can be used to deliver the invention in vivo or can be added to a composition comprising RNA and/or lipids administered to an animal.

“Treatment” or “treating” refers to both therapeutic treatment and to prophylactic or preventative measures. Those in need of treatment include those already with the disorder as well as those prone to have the disorder, or those in whom the disorder is to be prevented.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal (domestic (cat, dog) or farm animal (livestock, horse, cow), mammal, or human, who will benefit from the method of this invention.

A disease, condition, or disorder is “alleviated” if the severity of a symptom of the disease or disorder, the frequency with which such a symptom is experienced by a subject, or both, are reduced.

As used herein, an “instructional material” includes a publication, a recording, a diagram, or any other medium of expression which can be used to communicate the usefulness of the of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified invention or be shipped together with a container. Alternatively, the instructional material may be shipped separately from the container with the intention that the instructional material and the invention be used cooperatively by the recipient.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### MAPT

The tau proteins (abbreviated from tubulin associated unit) form a group of six highly soluble protein isoforms produced by alternative splicing from the gene MAPT (microtubule-associated protein tau). They have roles primarily in maintaining the stability of microtubules in axons and are abundant in the neurons of the central nervous system (CNS), where the cerebral cortex has the highest abundance. They are less common elsewhere but are also expressed at very low levels in CNS astrocytes and oligodendrocytes.

Pathologies and dementias of the nervous system such as Alzheimer's disease and Parkinson's disease are associated with tau proteins that have become hyperphosphorylated insoluble aggregates called neurofibrillary tangles. The tau proteins were identified in 1975 as heat-stable proteins involved for microtubule assembly, and since then they have been characterized as intrinsically disordered proteins.

In humans, the MAPT gene for encoding tau protein is located on chromosome 17q21, containing 16 exons (Genome assembly GRCh38.p14; GCF_000001405.40 and GCA_000001405.29, incorporated herein in its entirety). The major tau protein in the human brain is encoded by 11 exons. Exons 2, 3 and 10 are alternatively spliced, which leads to the formation of six tau isoforms. In the human brain, tau proteins constitute a family of six isoforms with a range of 352-441 amino acids. Tau isoforms are different in having either zero, one, or two inserts of 29 amino acids at the N-terminal part (exons 2 and 3) and three or four repeat-regions at the C-terminal part (exon 10). Thus, the longest isoform in the CNS has four repeats (R1, R2, R3 and R4) and two inserts (441 amino acids total), while the shortest isoform has three repeats (R1, R3 and R4) and no insert (352 amino acids total).

Six tau isoforms exist in human brain tissue, and they are distinguished by their number of binding domains. Three isoforms have three binding domains and the other three have four binding domains. The binding domains are located in the carboxy terminus of the protein and are positively charged (allowing it to bind to the negatively charged microtubule). The isoforms with four binding domains are better at stabilizing microtubules than those with three binding domains. Tau is a phosphoprotein with 79 potential serine (Ser) and threonine (Thr) phosphorylation sites on the longest tau isoform.

### 4R-Tau

Tau is a microtubule-binding protein expressed in neurons, and the equal ratios between 4-repeat (4R) and 3-repeat (3R) isoforms are maintained in normal adult brain function. Tau amino acid sequence and regions of the longest 4R tau isoform (2N4R) consisting of 441 amino acids. Six isoforms differ by differential inclusion of N1, N2, and R2. The microtuble-binding repeat region (MTBR) of 4R tau isoforms comprise all four repeats (R1-R4) while that of 3R tau isoforms are missing R2.). The inclusion or exclusion of exon 10 gives rise to 4-repeat (4R) and 3-repeat (3R) tau, respectively, and these two isoform families are expressed at an approximately 1:1 ratio in the healthy adult human brain. Dysregulation of 3R:4R ratio causes tauopathy.

Human mRNA accession numbers for MAPT include, but are not limited to, NM_001123066; NM_001123067; NM_001203251; NM_001203252; and NM_005910, each of which are incorporated herein by reference for the mRNA sequence of MAPT. Human protein accession numbers for MAPT include, but are not limited to, NP_001116538; NP_001116539; NP_001190180; NP_001190181 and NP_005901, each of which are incorporated herein by reference for the protein sequence of MAPT.

Exemplary sequence of 4R-tau:
MAEPRQEFEV MEDHAGTYGL GDRKDQGGYT MHQDQFDDTD AGLKESPLQT PTEDGSEEPG SETSDAKSTP TAEDVTAPLV DEGAPGKQAA AQPHTE [symbol] EG TTAEEAG [symbol] D TPSLEDEAAG HVTQARMVSK SKDGTGSDDK KAKGADGKTK [symbol] TPRGAAPP GQKGQANATR [symbol] AKTPPAPK TPPSSGEPPK SGDRSGYSSP GSPGTPGSRS RTPSLPTPPT REPKKVAVVR TPPKSPSSAK SRLQTAPVPM PDLKNVKSK [symbol] GSTENLKHQP GGGKVQ [symbol] K KLDLSNVQSK CGSKDN [symbol] HV [symbol] GGGSVQ [symbol] Y KPVDLSKVTS KCGSLGN [symbol] H KPGGGQVEVK SEKLDFKDRV QSK [symbol] SIDN [symbol] THVPGGGNKK [symbol] THKTFRE NAKAKTDHGA E [symbol] YKSPVVS GDTSPRHLSN VSSTGS [symbol] MV DSPQLATLAD EVSASLAKQG L (SEQ ID NO: 3), or sequence with at least 55%, 60%, 70%, 80%, 90%, 95%, 96%, 97%, 98%, 99% sequence identity thereto. Identity/alignment may be ascertained or conducted using, for example, the homology alignment algorithm of Needleman and Wunsch, J. Mol. Biol. 48:443-53 (1970).

Phosphorylation of tau is regulated by a several kinases, including PKN, a serine/threonine kinase. When PKN is activated, it phosphorylates tau, resulting in disruption of microtubule organization. Phosphorylation of tau is also developmentally regulated. For example, fetal tau is more highly phosphorylated in the embryonic CNS than adult tau. The degree of phosphorylation in all six isoforms decreases with age due to the activation of phosphatases.

The accumulation of hyperphosphorylated tau in neurons is associated with neurofibrillary degeneration. The actual mechanism of how tau propagates from one cell to another is not well identified. Also, other mechanisms, including tau release and toxicity, are unclear. As tau aggregates, it replaces tubulin, which in turn enhances fibrillization of tau. Several propagation methods have been proposed that occur by synaptic contact such as synaptic cell adhesion proteins, neuronal activity and other synaptic and non-synaptic mechanisms. The mechanism of tau aggregation is still not completely elucidated, but several factors favor this process, including tau phosphorylation and zinc ions.

iPSC(s)

Induced pluripotent stem cells (also known as iPS cells or iPSCs) are a type of pluripotent stem cell that can be generated directly from a somatic cell, such as fibroblast cell. The iPSC technology was pioneered by Shinya Yamanaka and Kazutoshi Takahashi in Kyoto, Japan, who together showed in 2006 that the introduction of four specific genes (named Myc, Oct3/4, Sox2 and Klf4), collectively known as Yamanaka factors, encoding transcription factors could convert somatic cells into pluripotent stem cells.

iPSCs are typically derived by introducing products of specific sets of pluripotency-associated genes, or “reprogramming factors”, into a given cell type. The original set of reprogramming factors (also dubbed Yamanaka factors) are the transcription factors Oct4 (Pou5f1), Sox2, Klf4 and cMyc. While this combination is most conventional in producing iPSCs, each of the factors can be functionally replaced by related transcription factors, miRNAs, small molecules, or even non-related genes such as lineage specifiers. Pro-mitotic factors such as C-MYC/L-MYC or repression of cell cycle checkpoints, such as p53, are conduits to creating a compliant cellular state for iPSC reprogramming. For example, a different set of factors, Oct4, Sox2, Nanog, and Lin28, can also be used (Yu J, et al. (2007). “Induced pluripotent stem cell lines derived from human somatic cells”. Science. 318 (5858): 1917-20).

### Expression Systems

Recombinant expression of nucleic acids (or inhibitory nucleic acids) is can be accomplished using a vector, such as a plasmid. The vector can include a promoter operably linked to nucleic acid segment encoding one or more proteins of interest. In another example, a vector can include a promoter operably linked to nucleic acid segment that encodes an inhibitory nucleic acid.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing a protein of interest and/or inhibitory nucleic acids can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, promoter, or nucleic acid refers to an expression cassette, expression vector, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of proteins of interest or inhibitory nucleic acid molecules therefor from an expression cassette or expression vector can be controlled by any promoter capable of expression in prokaryotic cells or eukaryotic cells. Examples of prokaryotic promoters that can be used include, but are not limited to, SP6, T7, T5, lac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if the gene has been delivered to the cell and once delivered is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the nucleic acid molecules, expression cassette and/or vectors encoding proteins of interest therefor can be introduced to a cell by any method including, but not limited to, calcium-mediated transformation, electroporation, microinjection, lipofection, particle bombardment and the like. The cells can be expanded in culture and then administered to a subject, e.g., a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

### CRISPR/Cas

In some cases, clustered regularly interspaced short palindromic repeats (CRISPR)/CRISPR-associated (Cas) systems can be used to create one or more modifications. Such CRISPR modifications can reduce or activate the expression or functioning of gene products. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6: Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

A CRISPR guide RNA can be used that can target a Cas enzyme ((e.g., Cas9, Cas12, Cas13, Cpf1, and the like)) to the desired location in the genome, where it can cleave the genomic DNA for generation of a genomic modification. This technique is described, for example, by Mali et al. Science 2013 339:823-6: which is incorporated by reference herein in its entirety. Kits for the design and use of CRISPR-mediated genome editing are commercially available, e.g., the PRECISION X CAS9 SMART NUCLEASE™ System (Cat No. CAS900A-1) from System Biosciences, Mountain View, CA.

The guide RNAs and nuclease can be introduced via one or more vehicles such as by one or more expression vectors (e.g., viral vectors), virus like particles, ribonucleoproteins (RNPs), via nanoparticles, liposomes, or a combination thereof. The vehicles can include components or agents that can target particular cell types (e.g., antibodies that recognize cell-surface markers), facilitate cell penetration, reduce degradation, or a combination thereof.

### Inhibitory Nucleic Acids

The expression of one or more proteins can be inhibited, for example by use of an inhibitory nucleic acid that specifically recognizes a nucleic acid that encodes the protein.

An inhibitory nucleic acid can have at least one segment that will hybridize to a nucleic acid of interest under intracellular or stringent conditions. The inhibitory nucleic acid can reduce expression of nucleic acid of interest. A nucleic acid may hybridize to a genomic DNA, a messenger RNA, or a combination thereof. An inhibitory nucleic acid may be incorporated into a plasmid vector or viral DNA, or it may not be. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a nucleic acid interest. Such an inhibitory nucleic acid may be completely complementary to a segment of an endogenous nucleic acid (e.g., an RNA). Alternatively, some variability is permitted in the inhibitory nucleic acid sequences relative to the sequences of interest (e.g., pathway inhibitor). An inhibitory nucleic acid can hybridize to a nucleic acid of interest under intracellular conditions or under stringent hybridization conditions and is sufficiently complementary to inhibit expression of the endogenous nucleic acid of interest. Intracellular conditions refer to conditions such as temperature, pH and salt concentrations typically found inside a cell, e.g., an animal or mammalian cell. One example of such an animal or mammalian cell is a neuronal cell. Another example of such an animal or mammalian cell is a differentiated neural cell derived from an iPSC. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to the coding sequence of interest, each separated by a stretch of contiguous nucleotides that are not complementary to adjacent coding sequences, can inhibit the function of one or more nucleic acids. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g., a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of a target nucleic acid such that translation of the encoded target polypeptide is reduced. SiRNAs mediate post-transcriptional gene silencing in a sequence-specific manner. See, for example, website at invitrogen.com/site/us/en/home/Products-and-Services/Applications/mai.html. Once incorporated into an RNA-induced silencing complex, si RNA mediate cleavage of the homologous endogenous mRNA transcript by guiding the complex to the homologous mRNA transcript, which is then cleaved by the complex. The siRNA may be homologous and/or complementary to any region of the target transcript. The region of homology may be 30 or 40 nucleotides or less in length, such less than 25 nucleotides, and such as about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of targets. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected about 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3's end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target nucleic acids.

An inhibitory nucleic acid may be prepared using available methods, for example, by expression from an expression vector encoding a complementarity sequence of the nucleic acids described herein. Alternatively, it may be prepared by chemical synthesis using naturally occurring nucleotides, modified nucleotides or any mixture of combination thereof. In some embodiments, the nucleic acids described herein are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, nucleic acids can be peptide nucleic acids that have peptide bonds rather than phosphodiester bonds.

Naturally occurring nucleotides that can be employed in the nucleic acids include the ribose or deoxyribose nucleotides adenosine, guanine, cytosine, thymine and uracil. Examples of modified nucleotides that can be employed in the nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-Soxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, inhibitory nucleic acids described herein may include modified nucleotides, as well as natural nucleotides such as combinations of ribose and deoxyribose nucleotides. The inhibitory nucleic acids and may be of same length as wild type. The inhibitory nucleic acids described herein can also be longer and include other useful sequences. In some embodiments, the inhibitory nucleic acids described herein are somewhat shorter. For example, inhibitory nucleic acids described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

### Assay/Screen for Drug Development

Methods are also described herein for evaluating whether test agents can inhibit or treat the formation of Tau bundles/inclusions. Neural cells can be evaluated for susceptibility to treatment with candidate compounds, such as small molecules.

For example, one method includes screening for compounds that can inhibit formation of Tau bundles/inclusions comprising contacting the neuronal cells described herein Tau fibrils and a test agent; detecting the presence or absence of Tau bundles/inclusions, wherein the absence of Tau bundles/inclusions correlates with the test agent being a compound that inhibits formation of Tau bundles/inclusions.

### Compositions

The invention also relates to compositions containing one or more active agents. Such active agents can be a polypeptide, a nucleic acid encoding a polypeptide (e.g., within an expression cassette or expression vector), a modified cell, an inhibitory nucleic acid, a small molecule, a compound identified by a method described herein, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form.

In some embodiments, the active agents of the invention (e.g., polypeptide, a nucleic acid encoding a polypeptide (e.g., within an expression cassette or expression vector), an antibody, an inhibitory nucleic acid, a small molecule, a compound identified by a method described herein, modified cells, or a combination thereof), are administered in a “therapeutically effective amount.” Such a therapeutically effective amount is an amount sufficient to obtain the desired physiological effect, such a reduction of at least one symptom of disease.

For example, active agents can reduce the symptoms of disease by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%. For example, the active agents may Tau bundles/inclusions or the formation of Tau bundles/inclusions by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, or nucleic acid chosen for administration, the disease, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, ribonucleoprotein complexes, and other agents are synthesized or otherwise obtained, purified as necessary or desired. These small molecules, compounds, polypeptides, nucleic acids, expression cassettes, ribonucleoprotein complexes, and other agents can be suspended in a pharmaceutically acceptable carrier and/or lyophilized or otherwise stabilized. The small molecules, compounds, polypeptides, nucleic acids, expression cassettes, ribonucleoprotein complexes, other agents, and combinations thereof can be adjusted to an appropriate concentration, and optionally combined with other agents. The absolute weight of a given small molecule, compound, polypeptide, nucleic acid, ribonucleoprotein complex, and/or other agents included in a unit dose can vary widely. For example, about 0.01 to about 2 g, or about 0.1 to about 500 mg, of at least one molecule, compound, polypeptide, nucleic acid, ribonucleoprotein complexes, and/or other agent, or a plurality of molecules, compounds, polypeptides, nucleic acids, ribonucleoprotein complexes, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the nature of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

While the active agent(s) and/or other agents can sometimes be administered in an oral dosage form, that oral dosage form can be formulated so as to protect the small molecules, compounds, polypeptides, nucleic acids, expression cassettes, ribonucleoprotein complexes, and combinations thereof from degradation or breakdown before the small molecules, compounds, polypeptides, nucleic acids encoding such polypeptides, expression cassettes, ribonucleoprotein complexes, and combinations thereof provide therapeutic utility. For example, in some cases the small molecules, compounds, polypeptides, nucleic acids encoding such polypeptide, expression cassettes, ribonucleoprotein complexes, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The present description is further illustrated by the following examples, which should not be construed as limiting in any way. The contents of all cited references (including literature references, issued patents, published patent applications as cited throughout this application) are hereby expressly incorporated by reference.

### Example

### Introduction

Human iPSC-derived neurons, especially those derived from mutation-carrier patients, are invaluable in modeling neurological diseases, including tauopathies (Karch et al., 2019; Paonessa et al., 2019). Combined with CRISPR-Cas9 technology, iPSC-derived neuronal platforms enable isogenic controls for disease modeling (Sohn et al., 2019) and functional genomics to identify disease modifiers (Tian et al., 2019). However, iPSC-derived neurons express very low levels of 4R-Tau even after extended periods of culture and are thus unsuitable to model 4R tauopathy, such as PSP (Sposito et al., 2015; Verheyen et al., 2015). The low levels of exon-10-containing tau also limit their relevance in modeling the dominant familial FTLD-tau mutations located in exon 10 (Paonessa et al., 2019). Moreover, it has been difficult to capitulate robust Tau aggregation in human iPSC-derived neurons. While no insoluble Tau aggregates were observed in MAPT-P301L or MAPT-IVS10+16 iPSC neurons (Paonessa et al., 2019), limited Tau inclusions were observed in the processes after 120 days. One likely contributing factor lies in modeling a condition that would require many years in aging neurons in young iPSC-neurons in culture for weeks, while another factor is the lack of 4R Tau in iPSC-derived neurons (Capano et al., 2022).

Provided herein is the establishment of a robust and scalable human iPSC 4R tauopathy model. New lines of iPSC-neurons to express 4R-tau and 4R-tau carrying the P301S MAPT mutation have been engineered. Upon seeding with Tau fibrils, it is shown that 4R-P301S neurons develop a progressive spread of Tau aggregation, aberrant neuronal activity, and endolysosomal pathway dysfunction. Using CRISPRi-based functional genomic screening, novel genetic modifiers and pathways are identified and a robust platform to discover therapeutic strategies is provided.

### Materials and Methods

### Cell Cultures

To generate a stable 4R-Tau (4R) iPSC line, human iPSCs described in a previous study (Wang et al., 2017; which is incorporated herein by reference in its entirety) that were engineered for inducible expression of Neurogenin2 (NGN2) transgene were integrated into the AAVS1 locus of WTC11 cells with a wild-type genetic background (Miyaoka et al., 2014). To force the inclusion of exon 10 that allows the predominant expression of 4R tau under the regulation of the endogenous MAPT transcription unit, a donor plasmid containing several mutations around exon 10 was made. The mutated exon 10 region and a puromycin selection cassette were flanked by the left and right homology arms obtained from WTC11 MAPT genomic sequences. Two sgRNAs were selected based on the IDT CRISPR design online tool. Two guide RNAs and Cas9 protein (IDT) were incubated for 10 min at room temperature. RNP was co-electroporated with 1.8 μg of donor DNA into WTC11 iPSCs (0.3×106) using the Lonza Nucleofector system (Lonza). Cells were then seeded in a 12-well plate. After 48 hours, transfected cells were selected with puromycin for 5 days. Knock-in clones were isolated by splitting single cells into 96-well plates. To identify the knock-in monoclonal cells, primers were designed to flank the outside of the homology arms and to be on the transgene of the targeting vector. Genomic DNA was isolated from individual targeted clones grown on 96-well plates. Homologous recombination events were identified by two simple PCR screenings. The PCR products from the positive clones were validated by Sanger sequencing. Non-specific integration and off-target were performed by PCR with the genomic DNA samples, followed by Sanger sequencing. Homozygous clones were further characterized by PCR and expanded for future use.

To excise the FRT-flanked puromycin cassette from the targeted alleles, 2 μg of pCDH-EF1-FLPe DNA was electroporated into 4R iPSCs (0.3×10⁶) using the Lonza Nucleofector system (Lonza). Cells were then seeded in a 12-well plate. After 48 hours, FRT cassette-deleted clones were isolated by splitting single cells into 96-well plates. To characterize the deletion driven by FLP-mediated recombination, primers flanking the FRT cassette were designed. Genomic DNA was isolated from individual clones grown on 96-well plates. After FLP recombination, the FRT-EF1-Puro-T2A-eGFP-FRT cassette was excised, leaving a single FRT that was identified by simple PCR screening. The excised locus yielded a 734-bp fragment, whereas the original locus containing the FRT cassette showed a 3.1-kb fragment. The 734-bp PCR products from the homozygous clones were validated by Sanger sequencing. Homozygous clones were further characterized by PCR and expanded for future use.

To further generate a stable disease-associated MAPT-P301S (4R-P301S) model, a long single-stranded DNA (ssDNA) donor template was designed to introduce C>T to obtain the intended point mutation that leads to the change of proline 301 to serine on Tau. The point mutation was flanked by 60-bp left and right homology arms that were obtained from the WTC11 genomic sequence surrounding the mutation. A sgRNA was designed at the mutated region to prevent recutting after homology-directed recombination. RNP and ssDNA (Ultramer DNA oligos, IDT) were electroporated into 4R iPSCs and selected as described above. Homologous recombination events were identified by PCR with two primers outside of the homology arms, followed by Sanger sequencing to determine the integration of the correct mutations and the absence of any additional unwanted mutations surrounding the site. Homozygous clones were further characterized by PCR and expanded for future use.

To knock out MAPT in the NGN2 iPSC genome, two sgRNAs were designed to delete a ˜1.8-kb fragment on the MAPT locus. One sgRNA specifically recognized the upstream of the MAPT promoter, and another sgRNA specifically recognized the exon containing the ATG start codon of the MAPT gene. RNP was electroporated into NGN2 iPSCs as described above. To validate the deletion, primers flanking the outside of the two sgRNAs target sequences were designed, and positive clones were screened by PCR. Homozygous clones were further characterized by PCR with three primers, two primers outside of the two sgRNAs target sequences, and one primer within the deleted region. The deletion and locus integrity were validated by Sanger sequencing of the PCR products of the homozygous clones. Homozygous MAPT KO clones were expanded for future use.

To generate a stable line expressing endogenous 4R-P301S-HaloTag-Tau fusion protein, a donor plasmid containing HaloTag cDNA fused with the exon containing ATG start codon of the MAPT gene was made. The donor construct consists of HaloTag-5′ Tau and a puromycin selection cassette that were flanked by left and right homology arms obtained from WTC11 MAPT genomic sequences. An sgRNA was selected using the IDT CRISPR design online tool. RNP and 1.8 μg of donor DNA were electroporated into 4R-P301S iPSCs using the Lonza Nucleofector system (Lonza) and selected as described. Integration of HaloTag-5′ Tau at the target locus was verified by two PCRs using primers flanking the outside of the homology arms and on the transgene of the donor construct. The PCR products from the positive clones were validated by Sanger sequencing. Non-specific integration was identified. Homozygous clones were further characterized by PCR and expanded for future use.

To knock in the stable CRISPRi constitutive system into 4R-P301S iPSC genome, 0.3×10⁵ iPSCs were electroporated with 0.5 μg of each TALEN DNA plasmid and 1 μg of pC13N-dCas9-BFP-KRAB (Addgene, 127968) donor DNA using the Lonza Nucleofector system (Lonza) and selected as described. Cells were then seeded in a 12-well plate. After 48 hours, transfected cells were expanded and seeded in 6-well plates and further expanded. iPSCs were dissociated using Accutase to prepare single-cell suspensions, followed by single-cell sorting based on the BFP expression. Sorted cells were recovered in a 12-well plate for 2 days with ROCK inhibitor (Y-27632, Cayman chemicals) and then isolated by splitting single cells into 96-well plates. To identify the dCas9 clones, primers on the CLYBL locus flanking the outside of the homology arms and primers on the transgene of the targeting vector were designed. Genomic DNA was isolated from individual targeted clones grown on 96-well plates. Homologous recombination events were identified by two simple PCR screenings. The PCR products from the positive clones were validated by Sanger sequencing. Non-specific integration was identified and homozygous 4R-P301S-dCas9 clones were further characterized by PCR and expanded for future use.

To stably knock out the VPS29 gene in the 4R-P301S iPSC genome, two sgRNAs were designed to delete an ˜8-kb fragment on the VPS29 locus. One sgRNA specifically recognized the upstream of the VPS29 promoter, and another sgRNA specifically recognized the downstream of exon 3. RNP was electroporated into 4R-P301S iPSCs as described. To screen the deletion, primers flanking the outside of the two sgRNAs target sequences were designed and positive clones were screened by PCR. With efficient CRISPR cutting and repair of DNA through non-homologous end joining, a ˜481-bp product was expected for the deletion. Positive clones were further characterized by PCR with three primers, two primers outside of the two sgRNAs target sequences, and one primer within the deleted region. Monoallelic targeting was indicated by the appearance of two distinct products of wild-type size and targeted size, the wild-type locus yielded a 207-bp fragment, whereas the targeted locus yielded a 400-bp product. A single fragment of 400 bp indicates that both alleles were targeted. The deletion and locus integrity were validated by Sanger sequencing of the PCR products of the homozygous clones. Homozygous clones were expanded for future use.

Pre-differentiation of human iPSCs into neurons was initiated by plating 1.5×10⁶ iPSCs in one well of Matrigel-coated 6-well plates with Knockout DMEM/F-12 medium containing doxycycline (2 μg/mL), N2 supplement, non-essential amino acids, brain-derived neurotrophic factor (10 ng/mL, Peprotech), neurotrophin-3 (10 ng/mL, PeproTech) and ROCK inhibitor (Y-27632, Cayman chemicals). The medium was replaced the next day without ROCK inhibitor, and pre-differentiation was maintained for a total of 3 days. On day 0, the pre-differentiated precursor cells were dissociated with Accutase and re-plated generally onto laminin-coated coverslips (Neuvitro) at 1.5×10⁵ cells/coverslip or 12-well tissue culture plates (Corning) at 3.8×10⁵ cells/well for the growth of neuronal cultures in Neurobasal Plus medium containing B27 supplement, Glutamax, BDNF (10 ng/mL) and NT3 (10 ng/mL) with doxycycline (2 μg/mL). Half of the medium was replaced on day 3, as well as on day 7 with the removal of doxycycline from the fresh medium. The medium volume was doubled on day 21. Thereafter, one-half of the medium was replaced with fresh medium weekly until the cells were collected. All human iPSC lines used in this work have been regularly tested for mycoplasma (Lonza) and karyotyped (MSK Molecular Cytogenetics Core).

### Human Subjects

The tissues used for this study were the mid-frontal cortices from brains of age-matched patients with AD (n=7, 3 females and 4 males). Samples were obtained from the University of Pennsylvania brain bank. All brains were donated after consent from the next-of-kin or an individual with legal authority to grant such permission.

### Preparation and Seeding of Recombinant K18-P301L Tau Fibrils

Myc-tagged K18-P301L Tau was expressed and purified as described (Mok et al., 2018). Briefly, protein expression was induced in Terrific Broth containing the chemical chaperone betaine (10 mM) and IPTG (500 μM) for 3 hours at 30° C. Tau was purified via the following major steps: mechanical lysis, boiling, centrifugation, and cation exchange. Purified Tau fractions were dialyzed into aggregation assay buffer (PBS pH 7.4, 2 mM DTT). To minimize potential endotoxin contamination, purified Tau was incubated with poly(epsilon-lysine)-conjugated resin (Pierce), and then tested post-treatment for endotoxin levels using the ToxinSensor™ Chromogenic LAL Endotoxin Assay Kit (Genscript). Endotoxin levels of tau were <0.1 EU/mL at working concentrations. Purified Tau aliquots were stored at −80° C. prior to aggregation. To induce Tau aggregation, 88 μg/mL of freshly prepared heparin sodium salt (Santa Cruz Biotechnology) was added to K18-P301L Tau (20 μM) in aggregation assay buffer. Aggregation was carried out in low-retention 1.7 mL microcentrifuge tubes at 37° C. with shaking at 800 rpm for 24 hours. Aggregated Tau was isolated by centrifugation at 100,000×g for 1 hour at 4° C. Pelleted Tau aggregates were resuspended in PBS (pH 7.4) with sterile plastic pestles and stored in low-binding tubes (CoStar) at −80° C. Tau fibril preparations were retested to confirm endotoxin levels <0.1 EU/mL at working concentrations. The concentration of aggregated Tau was quantified by Pierce BCA assay. Fibrils were thawed on ice and mixed by pipetting, and the volume needed for seeding was transferred to 100 μL of sterile DPBS. The fibrils were sonicated at 4° C., 10 minutes on/off, 30-second pulse, and amplitude of 40% using a water bath sonicator (EpiSonic 2000, EpigenTek). The fibrils were added to appropriate media volume in wells with neurons to achieve a 1.5 or 3 μg/mL final concentration.

### Western Blot

Human iPSC-derived neurons were washed twice with cold DPBS, centrifuged at 300 g for 5 minutes at 4° C. and homogenized in cold N-PER Neuronal Protein Extraction Reagent (Thermo Fisher) or cold RIPA lysis buffer (Thermo Fisher) supplemented with protease inhibitor cocktail (Millipore Sigma), phosphatase inhibitor cocktail (MilliporeSigma) and deacetylase inhibitors, including nicotinamide (Millipore Sigma) and trichostatin A (Millipore Sigma). N-PER and RIPA lysates were incubated on ice for 10 minutes, centrifuged at 14,000 g for 10 minutes at 4° C., and the supernatants were collected, according to the manufacturer's instructions. For Triton X-100 soluble/insoluble fractionation, cell pellets were resuspended in cold lysis buffer containing 50 mM Tris pH 7.6, 150 mM NaCl, 1% (v/v) Triton-X 100, protease inhibitor cocktail (Roche), phosphatase inhibitor cocktail (Sigma), and deacetylase inhibitors, including nicotinamide and trichostatin A. Samples in lysis buffer were incubated on ice for 30 minutes, centrifuged at 20,000 g for 30 minutes at 4° C., and the supernatant was collected as a Triton-soluble fraction. Triton-insoluble pellets were further resuspended in lysis buffer containing 50 mM Tris, pH 7.6, 150 mM NaCl, 5% (w/v) sodium dodecyl sulfate (SDS), protease inhibitor cocktail (Roche), phosphatase inhibitor cocktail (Sigma), nicotinamide and trichostatin A using one-third of the final volume of the Triton-soluble fraction. Insoluble samples were sonicated at 16° C. for 4 minutes on/off with 2-second pulses and 20% amplitude using a water bath sonicator (EpiSonic 2000, EpigenTek), centrifuged at 20,000 g for 30 minutes at 20° C. and the supernatant was collected as Triton-insoluble fraction. Protein concentration was determined using a Pierce BCA Protein Assay Kit (Thermo Fisher) for all samples except the Triton-insoluble fractions. Equal amounts of protein for each sample were run on a 4-12% SDS-PAGE gel (Invitrogen). Nitrocellulose membranes (GE Healthcare) were used for the transfer of proteins, and blots were blocked with 5% milk in TBS-Tween. Primary and secondary antibodies were diluted and incubated in the blocking solution. Primary antibodies used included mouse anti-tau (HT7, Thermo Fisher), anti-Tau 3-repeat isoform (RD3, Sigma), anti-Tau 4-repeat isoform (ET3, Sigma), anti-phosphorylated Tau (AT8, Thermo Fisher), anti-phosphorylated PHF Tau (AT180, Thermo Fisher), anti-phosphorylated PHF Tau (AT270, Thermo Fisher), and rabbit anti-UFM1 (Abcam), anti-GAPDH (GeneTex). Secondary HRP antibodies (Millipore) and chemiluminescence (BioRad) were used for detection of immunoblotting and bands in immunoblots were quantified by intensity using ImageLab (BioRad) or FIJI (NIH) software.

### Immunocytochemistry and Imaging

For human iPSCs and iPSC-derived neurons, cells were fixed in 4% paraformaldehyde (Electron Microscopy Sciences) diluted in PBS for 15 minutes and washed three times for 5 minutes in DPBS with Mg²⁺ and Ca²⁺. Cells were permeabilized in 0.1% Triton X-100 diluted in DPBS for 10 minutes and then placed in a blocking solution containing 0.1% Triton X-100 and 5% goat serum in DPBS for 1 hour at room temperature. Primary antibodies diluted in blocking solution were added to the cells overnight at 4° C. and then washed three times for 5 minutes with DPBS. Primary antibodies used included: chicken anti-MAP2 (Novus Biologicals), mouse anti-conformationally abnormal Tau (MC1, Peter Davies), anti-phosphorylated Tau (AT8, Thermo Fisher), anti-SSEA (Abcam), anti-TRA-1-60 (Abcam), anti-TRA-1-81 (Abcam), and rabbit anti-oligomeric Tau (TTC18, Rakez Kayed), anti-UFM1 (Abcam), anti-GFP (Abcam), anti-OCT4 (Abcam), NANOG (Abcam), and SOX2 (Cell Signaling). Secondary antibodies diluted in blocking solution included donkey anti-mouse, anti-rabbit, anti-chicken and anti-human antibodies conjugated with Alexa Fluor 488, Alexa Fluor 568, or Alexa Fluor 646 fluorophores (1:500, Life Technologies), and they were added to the cells for 1 hour at room temperature. The coverslips were washed three more times for 5 min with DPBS before being mounted on slides with Vectashield containing DAPI (Thermo Fisher). Images were acquired using Apotome (Zeiss), Keyence (BZ-X710), or LSM 880 Laser Scanning Confocal Microscope (Zeiss) and processed with Zen 3.2 software. The settings used for image acquisition were selected to keep the majority of the brightest pixel intensities from reaching saturation. Quantification was performed using FIJI (NIH) software.

For human AD brain tissue, sections were formalin-fixed and paraffin-embedded into glass slides. Slides were placed for 10 minutes at 60° C. in an oven and then de-paraffinized by washing with xylene three times for 5 mins, 100% ethanol two times for 2 mins, 95% Ethanol for 2-5 mins, and deionized water 3 times for 2 minutes. For antigen retrieval, the slides were placed in working Tris-EDTA buffer (10 mM Tris base, 1 mM EDTA solution, 0.05% Tween 20, pH 9.0) and placed in a pressure cooker (Cuisinart) at high pressure for 15 minutes. The slides were cooled to RT and washed with cool deionized water three times for 2 minutes. Sections were washed with 1×PBS three times for 2 minutes and incubated with 1× TrueBlack in 70% Ethanol for 30 seconds. The reaction was stopped by placing the slides in 1×PBS and further washing with 1×PBS three times for 2 minutes. Sections were blocked with 5% donkey serum in 1×PBS for 1 hour and incubated with UFM1 (Abcam 1:100) and MC1 (1:1000) diluted in 1% donkey serum in 1×PBS solution overnight in a humidified slide chamber. The following day, the slides were washed in 1×PBS three times for 2 minutes and incubated with AF 488 donkey anti-mouse (1:500) and AF 568 donkey anti-rabbit (1:500) diluted in 1% donkey serum in 1×PBS solution for 1 hour in a humidified slide chamber. The slides were washed in 1×PBS three times for 2 minutes and incubated with Hoescht 33342 diluted (1:1000) in 1×PBS for 15 minutes. The slides were washed in 1×PBS three times for 2 minutes and covered with Vectashield mounting medium without DAPI (Vector Labs). Five imaging fields in the gray matter of the same case were captured using a fluorescent microscope (Keyence BZ-X710), and UFM1 mean intensity of n=3 MC1+ and MC1-cells was quantified using NIS-Element Analysis software.

### Flow Cytometry

For flow cytometry, briefly, human iPSC-differentiated neurons were dissociated from the plate with papain solution (20 U/mL papain and 5 mM MgCl₂ in HBSS) at 37° C. for 30 mins. Papain was quenched with 3× volume DMEM with 10% FBS. Cells were fixed with zinc fixation buffer (0.1 M Tris-HCl with pH 6.5, 0.5% ZnCl₂, 0.5% Zn acetate and 0.05% CaCl₂) overnight at 4° C. The next day, samples were washed twice with TBS and resuspended in permeabilization buffer (10% donkey serum, 10% 10×TBS, 3% BSA, 1% glycine, 0.5% Tween-20) for 15 minutes. Primary antibodies are added into permeabilization buffer by pipetting up and down to separate cells into a single-cell suspension, and samples were incubated either at 4° C. overnight or at room temperature for 1 hour. The samples were incubated in permeabilization buffer with secondary antibodies at room temperature for 1 hour after washing twice in TBS. Samples were washed twice with TBS, analyzed with Attune NxT Flow Cytometer (Thermo Fisher) or LSRFortessa cell analyzer (BD), and the data were processed using FlowJo v10 software. To detect endogenous Tau inclusions, anti-MC1 (1:200, Peter Davies) primary antibody was used.

### Bulk RNA-Sequencing

4R and 4R-P301S iPSCs were differentiated into neurons on 12-well tissue culture plates, and half of the samples were seeded with 1.5 pg/ml K18 on D8 as described. Neurons were maintained and collected on D43. Total RNA was extracted from the samples using QuickRNA MicroPrep Kit (Zymo Research). After RNA isolation, total RNA integrity was checked using a 2100 Bioanalyzer (Agilent Technologies), and concentrations were measured by Nanodrop (Thermo Fisher). Preparation of the RNA sample library and RNA-seq were performed by the Genomics Core Laboratory at Weill Cornell Medicine using Illumina Stranded mRNA Sample Library Preparation kit (Illumina), according to the manufacturer's protocol. The normalized cDNA libraries were pooled and sequenced on NovaSeq 6000 (Illumina) with pair-end 100 cycles. The raw sequencing reads in BCL format were processed through bcl2fastq 2.19 (Illumina) for FASTQ conversion and demultiplexing. Paired-end reads were cleaned by trimming adapter sequences and low-quality bases using cutadapt v1.18 (Martin, 2011) and aligned and mapped to the human genome (GRCh38) using STAR v2.5.2 (Dobin et al., 2013). The transcriptome reconstruction was performed by Cufflinks v2.1.2, and the abundance of transcripts was measured with Cufflinks in Fragments Per Kilobase of exon model per Million mapped reads (FPKM) (Trapnell et al., 2010). Raw read counts per gene were extracted using HTSeq-count v0.11.2 (Anders et al., 2015). DEGs were identified using the RStudio DESeq2 package (Love et al., 2014), and plots were generated with the ggplot2 package v3.4.1. To find enriched annotations within hit genes, Gene Ontology analysis was performed using Gene Set Enrichment Analysis software on the four conditions using the Molecular Signatures Database C5 ontology gene sets (Liberzon et al., 2011; Subramanian et al., 2005).

### Single-Cell RNA-Sequencing

4R-P301S iPSCs were differentiated into neurons, K18-seeded, and matured to D39 as described. Neuronal dissociation was performed based on established protocols (Jerber et al., 2021; Tian et al., 2019). Neurons were washed twice with 1×DPBS (Thermo Fisher) before adding a 1:1 Accutase (Thermo Fisher Scientific) and 1×DPBS solution containing 20U/ml papain (Worthington Biochemical) and 50 μg/mL DNase I (Worthington Biochemical). The cells were incubated at 37° C. for up to 35 minutes before adding four times the dissociation solution volume of quenching solution composed of DMEM/F12 (Thermo Fisher Scientific), 10% FBS (Thermo Fisher Scientific), 10 pM ROCK inhibitor (Y-27632, Cayman chemicals) and 50 μg/mL DNase I (Worthington Biochemical) and transferring the neurons coming off as a layer to a 15-mL tube. Cells were centrifuged at 200×g for 4 minutes at RT, resuspended in quenching solution, gently dissociated using a P1000, and collected in a 15-mL tube capped with a 40-μm cell strainer (Corning), and washed three additional times in 1×DPBS containing 0.04% BSA (Sigma Aldrich). Single-cell suspensions were counted using an automated cell counter (TC20, BioRad).

### Transmission Electron Microscopy

Neurons were washed once in DPBS and fixed overnight at 4° C. with 2.5% glutaraldehyde and 4% PFA in 0.2 M sodium cacodylate buffer (pH 7.3) supplemented with 5% saturated aqueous solution of picric acid (Ito and Kamovsky, 1968). The next day, cells were washed three times for 10 minutes each with 0.1 M sodium cacodylate buffer (pH 7.3), postfixed in 1% osmium tetroxide and 1.5% potassium ferricyanide for 1 hour at room temperature, dehydrated in graded alcohols (50, 70, 85, 95%, and three times 100%) for 15 minutes at each concentration, and embedded in epoxy resin. Thin sections (65 nm) were cut on 200-mesh copper grids, stained with 1.5% uranyl acetate, followed by lead citrate (Venable and Coggeshall, 1965), and examined using a digital TEM (JSM1400, JEOL Ltd). Images were acquired using a Veleta 2 k×2 k charge-coupled device camera (Olympus-SIS) at low magnifications to capture the soma or dendrites and then at 150,000× to capture tau fibrils. Multilamellar bodies (MLBs) were identified using morphological criteria. Both the number of MLBs per soma and the total area of MLBs normalized to the soma area were calculated by manual contouring in Fiji (NIH).

### Calcium Imaging

4R-P301S-HaloTag iPSCs were differentiated into neurons on coverslips as described. Neurons were transduced with hSyn-jGCaMP8f lentivirus on D6. This lentiviral construct was made using the 3rd generation lentiviral plasmid FUGW (Addgene, 14883), where the PacI+EcoRI fragment was replaced by the hSyn-GCaMP8f fragment (Zhang et al., 2023). pGP-AAV-hSyn-jGCaMP8f-WPRE was a gift from GENIE Project (Addgene, 162376). The medium was changed on D7, and neurons were seeded with 1.5 pg/mL K18-tau fibrils. Neurons were maintained as described until imaging at 4 and 5 weeks old. On the day of imaging, JFX-549 HaloTag ligand (HHMI Janelia) was diluted into fresh maturation medium to 1 pM, and one-fifth of neuronal medium was replaced with diluted ligand (200 nM final ligand concentration). Cells were incubated at 37° C. for 15 minutes. An equal volume of medium was replaced with half-fresh medium, and half-conditioned medium from a control well. At the time of imaging, the coverslip was gently washed and placed into a glass-bottom chamber (RC-26G, Warner Instruments) containing Ca2+ imaging buffer (20 mM HEPES, 119 mM NaCl, 5 mM KCl, 2 mM MgCl₂, 30 mM glucose, 2 mM CaCl₂, pH 7.2-7.4). The temperature was maintained at 37° C. by a dual chamber heat controller (TC-344C, Warner Instruments). Fluorescence time-lapse images were collected on a Nikon FN1 microscope using a 60×, 1.0 NA objective (CFI APO 60XW NIR, Nikon) and a C-FL GFP filter cube. An X-CITE LED illuminator (Nikon) was used for excitation. Images were collected using an ORCA-Fusion CMOS camera (Hamamatsu) with 4×4 binning (576×576 pixel resolution, 16-bit grayscale depth, 0.43 m/pix) and NIS-Elements AR software (Nikon). HaloTag signal per field of view was collected using a C-FL-DS RED filter cube (Nikon). Exposure time was set to 20 milliseconds. For spontaneous activity, 4-5 fields per coverslip were acquired at 30 Hz for 2 minutes. After imaging spontaneous activity, one field per coverslip was imaged for 10 minutes at 20 Hz to minimize photo-bleaching immediately after 50 mM KCl perfusion.

### DREADDs Neuronal Silencing

4R-P301S-HaloTag iPSCs were differentiated into neurons on coverslips as described. Neurons were transduced with hSyn-hM4Di-mCherry lentivirus on D6. This lentiviral construct was made using the 3rd generation lentiviral plasmid FUGW (Addgene, 14883), where the PacI+EcoRI fragment was replaced by hSyn-hM4D(Gi)-mCherry. pAAV-hSyn-hM4D(Gi)-mCherry was a gift from Bryan Roth (Addgene, 50475; http://n2t.net/addgene). The medium was changed on D7, and neurons were seeded with 1.5 pg/mL K18-tau fibrils. Neurons were maintained as described. Beginning at D14, clozapine-N-oxide (CNO; Tocris) was included in weekly media changes for a final concentration of 10 pM. Neurons were fixed and stained with MC1 on D29. For MC1 quantification, stained neurons were imaged with a fluorescence microscope (Keyence BZ-X710). A 20× objective was used to acquire a series of 10 Z-stack images per coverslip. To validate the neuronal silencing effect of CNO by calcium imaging, 4R-P301S neurons were differentiated and transduced with hSyn-hM4Di-mCherry and hSyn-GCaMP8f lentivirus on D6. At D36, JFX-549 was added to neurons and prepared for imaging as described above. Exposure time was set to 20 milliseconds. Neurons were perfused with 10 μM CNO at 60 seconds and calcium traces in one field were recorded for 400 seconds.

### VAMP7DN Human Tau ELISA and Immunocytochemistry

For Human Tau ELISA, 4R and 4R-P301S iPSCs were differentiated on 12-well tissue culture plates as described previously. Neurons were seeded with 1.5 pg/mL K18 on D14. Two weeks after seeding on D28, the cell culture medium was removed and replaced with high KCl extracellular solution containing (in mM): 68.5 NaCl, 50 KCl, 10 HEPES, 20 glucose, 1 MgCl₂, 2.5 CaCl₂ at pH 7.4. The neurons were incubated with KCl extracellular solution for 30 minutes at 37° C. The extracellular solution was collected for ELISA analysis. The neurons were lysed with cold RIPA buffer (100 μL/well) and homogenized on ice with a handheld homogenizer for 2 minutes for each sample. Samples were centrifuged at 20,000 g for 10 minutes at 4° C. and the supernatants were collected. Protein concentrations for the extracellular solution and cell lysate supernatant were determined by BCA assay with Pierce BCA protein assay kit (Thermo Fisher Scientific) according to manufacture protocol on Synergy H1 microplate reader (Biotek). A concentration series of seven Tau-352 protein standards were prepared for human Tau antibody-coated plates with 96-wells (Human Tau (Total) ELISA Kit, Cat. No. KHB0042). The extracellular solution was diluted 1:100, and the lysate was diluted to 1:1000 for all samples. Each standard (100 μL) or sample (50 μL) plus Standard Diluent Buffer (50 μL) was added to individual blocked wells, tapped to mix, and covered and incubated for 2 hours at room temperature shaking at 100 rpm. The solution was thoroughly aspirated, and wells were washed 4× with 400 μL of 1× wash buffer. Then 100 μL human Tau (Total) biotin conjugate solution was added into each well except the chromogen blanks. The plate was covered and incubated for 1 hour at RT shaking at 100 rpm. The solution was thoroughly aspirated, and wells were washed as described above. Next, 100 μL of 1× streptavidin-HRP solution was added into each well except for the chromogen blanks. The plate was covered and incubated for 30 minutes at RT shaking at 100 rpm. The solution was thoroughly aspirated, and wells were washed as described above. Next, 100 μL of stabilized chromogen was added to each well, following 50 μL streptavidin-HRP solution (1:8000). The plate was incubated for 30 minutes at RT in the dark. Lastly, 100 μL of stop solution was added to each well, tapped to mix, and the plate was read at 450 nm. The amount of secreted Tau was quantified from the percent of extracellular Tau detected out of the total amount of intracellular and extracellular Tau for each well of culture.

For immunocytochemistry, 4R and 4R-P301S iPSCs were differentiated into neurons on coverslips as described. Neurons were transduced with GFP or GFP-VAMP7DN lentivirus on D13. pFUGW-eGFP and pFUGW-eGFP-VAMP72-120 were gifts from Manu Sharma (Xie et al., 2022) The medium was changed on D14, and neurons were seeded with 1.5 pg/mL of K18-Tau fibrils. Neurons were maintained as described, and fixed and stained with MC1 on D28. For MC1 quantification, stained neurons were imaged with a fluorescence microscope (Keyence BZ-X710). A 20× objective was used to acquire a series of 10 Z-stack images per coverslip.

### Lentivirus Generation

Generally, lentiviral particles were generated for pFUGW-hSyn-GCaMP8f, pFUGW-hM4Di-mCherry, pFUGW-eGFP, and pFUGW-eGFP-VAMP7DN as follows. HEK293T cells (ATCC) were thawed, passed, and expanded in T75 flasks with 15 mL of DMEM/F-12 (basal medium supplemented with 10% FBS and 1% penicillin/streptomycin). Cells were passed into 10-cm dishes (1:10), and 2 days later upon reaching ˜80% confluency, transfection mix for each plasmid was prepared in the following manner: 1 μg of transfer plasmid and 2 μg of second-generation packaging DNA mix containing psPAX (Addgene #12260) and pMD2.G (Addgene #12259)(molarity 1:1) were diluted into Opti-MEM I Reduced Serum Medium (GIBCO; Cat. No. 31985070); TransIT-LT1 (Mirus Bio, Cat. No. MIR6600) was diluted into Opti-MEM and incubated at room temperature for 5 minutes; the diluted DNA solution was added to the diluted Lipofectamine solution, inverted several times to mix, and incubated at room temperature for 10 minutes. Volumes depended on the number of 10-cm plates used according to the manufacturer's protocol. After incubation, the transfection solution was gently added dropwise to each 10-cm dish with HEK293T cells, and the plates were briefly and gently swirled to mix. At 48 hours, HEK293T medium was transferred into 50-mL conical tubes, and the plates were replaced with fresh medium. At 72 hours, HEK293T medium was collected again and combined with previously collected media. The supernatant was carefully transferred to a syringe fitted with a 0.45-μm PVDF filter to filter the virus-containing solution into a new 50-mL conical. Cold Lenti-X Concentrator (Takara; Cat. No. 631232) corresponding to one-third volume of the supernatant was added to the filtered solution, which was then mixed well and stored at 4C for 24 hours. After incubation, the solution was centrifuged at 4° C. for 45 minutes at 1,500 g, and the supernatant was decanted. The virus-containing pellet was resuspended in 200 μL of DPBS (ThermoFisher) and stored at −80° C. Lentiviral particles for single sgRNAs (non-targeting, TFRC, VPS29, LAMTOR5, UFM1) and shRNAs (UFM1, UBA) were generated as described above with the following modifications: third-generation packaging DNA mix containing pRSV-REV (Addgene #12253), pMDLg/pRRE (Addgene #12251), and pMD2.G (Addgene #12259) (mixed 1:1:1); 1:4 Lentivirus Precipitation Solution (Alstem, Cat. No. VC125) for concentration step; lentiviral media collection at 48 hours.

Sequences and Primers for shRNAs

| Gene | |
|---|---|
| | shRNA sequences |
| UBA5 | GGTAAGTTGCTACTCTTTGAT (SEQ ID NO: 4) |
| UFM1 | CCAATGATGGAATAGGAATAA (SEQ ID NO: 5) |
| UFBP1 | GCAAGTTCATCTACATAACCC (SEQ ID NO: 6) |
| Control | GCTTCGCGCCGTAGTCTTA (SEQ ID NO: 7) |
| | shRNA primers |
| UBA5 | gatccgGGTAAGTTGCTACTCTTTGATTCAAGAGAT |
| | CAAAGAGTAGCAACTTACCttttttg (SEQ ID |
| | NO: 8) |
| UFM1 | gatccgCCAATGATGGAATAGGAATAATCAAGAGTT |
| | ATTCCTATTCCATCATTGGttttttg (SEQ ID |
| | NO: 9) |
| UFBP1 | gatccgGCAAGTTCATCTACATAACCCTCAAGAGGG |
| | GTTATGTAGATGAACTTGCttttttg (SEQ ID |
| | NO: 10) |
| Control | gatccgGCTTCGCGCCGTAGTCTTATCAAGAGTAAG |
| | ACTACGGCGCGAAGCttttttg (SEQ ID NO: |
| | 11) |

Functional Validation of 4R-P301S-dCas9 iPSC Line

4R-P301S-dCas9 iPSCs were seeded at 5×10⁵ per well in six-well tissue culture plates with ROCK inhibitor. The following day at 24 hours, the medium was replaced with fresh medium, and iPSCs were transduced with single sgRNA lentiviruses (NTC, TFRC). The next day, a complete media change was performed. The MOI, quantified as the fraction of BFP-positive cells by flow cytometry, was ˜12%. The following day, 0.8 pg/mL puromycin selection (ThermoFisher) enriched sgRNA-expressing cells. On day 2 of selection, the cells were split 1:3 into six-well plates. After 2 more days of selection, the cells were assessed by flow cytometry (˜75% expressed high levels of BFP). The following day, the iPSCs were seeded for pre-differentiation and were further differentiated, seeded with 1.5 pg/mL of K18 on D7, and maintained as described. On D21, neurons were dissociated, resuspended, and blocked for 15 minutes with 1:20 human FC block (BD Biosciences; Cat. No. 564220) and then stained with 1:66 PE-Cy7 anti-human CD71 (TFRC) (BioLegend: Cat. No. 334112) for 30 minutes in the dark. Cells were washed with DPBS before analyzing them by flow cytometry using the LSRFortessa cell analyzer (BD). Flow cytometry data were analyzed using FlowJo (FlowJo, v.10.7.1); percent BFP+/CD71+ TFRC cells were normalized to those in the NTC samples; and data were plotted as fold-change using Prism 8 (GraphPad, v.9.4.1).

### Primary CRISPRi Screen

The CRISPRi custom Tau library with the top five sgRNAs per gene (Horlbeck et al., 2016) was packaged into lentivirus for transduction of iPSCs as follows. One 15-cm dish was seeded with 12×10⁶ HEK293T cells in 30 mL of DMEM/F-12 (basal medium supplemented with 10% FBS and 1% penicillin/streptomycin). The next day, the custom Tau library transfection mix was prepared in the following manner: 15 μg of Tau custom library plasmid and 15 μg of third-generation packaging DNA mix containing pRSV-REV (Addgene #12253), pMDLg/pRRE (Addgene #12251), and pMD2.G (Addgene #12259)(mixed 1:1:1) were diluted into 3 mL of Opti-MEM I Reduced Serum Medium (GIBCO; Cat. No. 31985070); 90 μL of TransIT-LT1 (Mirus Bio, Cat. No. MIR6600) was diluted into 3 mL of Opti-MEM and incubated at RT for 5 minutes; the diluted DNA solution was added to the diluted Lipofectamine solution, inverted several times to mix, and incubated at room temperature for 10 minutes. After incubation, half of the transfection solution was gently added dropwise to each 15-cm dish with HEK293T cells, and the plates were briefly and gently swirled to mix. Two days later, HEK293T media (approximately 30 mL) was transferred into a 50-mL conical tube. The supernatant was carefully transferred to a syringe fitted with a 0.45 μm PVDF filter to filter the virus-containing solution into a new 50 mL conical. Approximately 7.5 mL of cold Lentivirus Precipitation Solution (Alstem; Cat. No. VC125) was added to the filtered solution, which was then mixed well and stored at 4C for 24 hours. Following incubation, the solution was centrifuged at 4C for 30 min at 1,500 g, and the supernatant was decanted. The virus-containing pellet was resuspended in 1 mL mTeSR Plus medium and stored at −80 C.

For infection with the tau custom library, one T175 Matrigel-coated flask was seeded with 5×106 4R-P301S-dCas9 iPSCs in mTeSR Plus medium with ROCK inhibitor. The following day at 24 hours, the medium was replaced with 35 mL fresh medium plus 500 μL of the virus-containing medium was added to the cells. The next day, a complete media change was performed, adding 30 mL mTeSR Plus medium. Two days later, 0.5 pg/ml Puromycin selection (ThermoFisher) was added into the 30 mL medium, which was the medium volume and formulation used for puromycin treatment to enrich sgRNA-expressing cells. The following day, cells were disassociated and seeded in one T175 Matrigel-coated flask with 5×10⁶ cells in 30 mL mTeSR with ROCK inhibitor. The MOI, quantified as the fraction of BFP-positive cells by flow cytometry, was ˜30%, corresponding to a library representation of ˜250 cells per library element. Puromycin treatment was increased to 0.8 pg/ml for the following three days. At the end of treatment, cells were assessed by blue fluorescence/bright field microscopy (˜75% expressed high levels of BFP) and seeded for the MC1 screen as described below.

For the MC1 screen, 3 T175 Matrigel-coated flasks were each seeded with 12×10⁶ cells in 30 mL N2 Pre-Differentiation Medium and differentiated as previously described into 6 15-cm PDL-coated dishes (Corning), seeded 15×10⁶ precursor cells each. On D7, the cells were seeded with 3 μg/mL K18. On D19, cells from 5 15-cm dishes (cells in 1 dish died) were dissociated by papain, fixed, and stained with MC1 as described previously. Cells were FACS-sorted into 1 mL 30% BSA (Sigma) solution using a FACSAria II (BD) based on MC1, approximately ˜2 million MC1+ cells and ˜6 million MC1− cells, corresponding to a library representation of ˜1,000 cells per library element in MC1− group and ˜333 cells per library element in MC1+ group. The cells were pelleted at 200 g for 20 minutes, the supernatant was carefully removed and stored at −20° C. Genomic DNA was extracted with the NucleoSpin Blood L kit (Macherey Nagel; Cat. No. 740954.20). The sgRNA-encoding regions were amplified, pooled, and sequenced on a MiSeq sequencer (Illumina) with single read 65 cycles including 20% Phi× Sequencing Control DNA at the Genomics Core Laboratory at Weill Cornell Medicine. The raw sequencing reads in BCL format were processed through bcl2fastq 2.20 (Illumina) for FASTQ conversion and demultiplexing, based on previously described protocols (Gilbert et al., 2014; Kampmann et al., 2014; Tian et al., 2019).

### Primary Screen Validation

4R-P301S-dCas9 iPSCs were seeded at 5×10 per well in six-well tissue-culture plates with ROCK inhibitor. The following day at 24 hours, the medium was replaced with 2 mL of fresh medium, and iPSCs were transduced with single sgRNA lentiviruses (NTC, VPS29, LAMTOR5, UFM1). The next day, a complete media change occurred. The MOI, quantified as the fraction of BFP-positive cells by flow cytometry, was ˜-22%. The following day, 0.8 pg/mL puromycin selection (ThermoFisher) enriched sgRNA-expressing cells. On day 2 of selection, the cells were split 1:3 into six-well plates. After 2 more days of selection, the cells were assessed by flow cytometry (˜74% expressed high levels of BFP). The following day, the iPSCs were seeded for pre-differentiation and were further differentiated, seeded with 1.5 pg/mL of K18 on D7, and maintained as described. On D21, neurons were zinc-fixed and stained with antibodies for flow cytometry as described. The samples were analyzed LSRFortessa cell analyzer (BD), and the data were processed using FlowJo v10 software. The following primary antibody was used: anti-MC1 (1:200). NucRed Live 647 ReadyProbes Reagent was used to detect and gate on intact cells.

### VPS29−/− and UFMylation Cascade Phenotype Validation

For VPS29−/− validation, 4R-P301S and 4R-P301S;VPS29−/− iPSCs were seeded at the density of 1.2×10⁷ per plate in 10-cm plates in pre-differentiation medium. On day 0, pre-differentiated neurons were replated at the density of 4×10⁵ per well in poly-D-lysine (PDL) pre-coated 12-well plates (Corning) or at the density of 2×10⁵ per well on laminin pre-coated coverslips (Neuvitro) in PDL pre-coated 24-well plates (Corning) in maturation medium. On day 7, iPSCs differentiated neurons were treated with 1.5 pg/mL of K18 fibrils for 2 weeks. On day 21, neurons in 12-well plates were collected for flow cytometry analysis, and neurons on coverslips were fixed for immunocytochemistry. For UFMylation cascade validation, 4R-P301S-iPSCs were seeded and differentiated as described above. On day 3, lentivirus containing control shRNA and shRNAs for UFM1 and UBA5 were added to the culture medium. The virus-containing medium was replaced with fresh medium after 24 hours. On day 7, iPSCs differentiated neurons were treated with 5 μg/mL of K18 fibrils for 2 weeks. On day 21, neurons in 12-well plates were collected for qPCR analysis and flow cytometry analysis, and neurons on coverslips were fixed for immunocytochemistry.

For flow cytometry, the cells were cultured, dissociated, and analyzed as described. The following primary antibodies were used: anti-MC1 (1:200, Peter Davies) and anti-GFP (1:400, Abcam). For immunocytochemistry, neurons were fixed and stained as described and imaged with LSM 800 confocal microscope (Zeiss). Primary antibodies were used: anti-MC1 (1:1000 Peter Davies), anti-UFM1 (1:250, Abcam), and anti-GFP (1:2000, Abcam). For qPCR, RNA was extracted from neurons using Quick-RNA™ Microprep Plus Kit (Zymo Research), according to the manufacturer's protocol. cDNA was synthesized using the iScript Reverse Transcription Supermix for RT-qPCR (Bio-Rad) and real-time PCR was performed using SsoAdvanced Universal SYBR® Green Supermix (Bio-Rad). The thermal cycling conditions were 95° C. for 10 minutes, 45 cycles of 95° C. for 15 seconds, 60° C. for 30 seconds, and 72° C. for 30 seconds. Data were collected using the BCFX384 Touch Real-Time PCR Detection System (Bio-Rad). Each sample was tested in replicate. Gene expression fold changes were calculated by the ΔΔCT method.

### Statistical Analysis

For statistical analysis, GraphPad Prism 9.2.0 software or R Version 4.2.2 with packages were used. Data are shown as mean±SEM. For two sample comparison, an unpaired two-tailed Student's t-test was used to quantify the data. For three sample comparison, the one-way ANOVA was utilized, followed by a Dunnett post hoc test with multiple testing correction and to set up a control. For comparison of resistant and non-resistant cell lines, a one-way ANOVA and a Tukey test with multiple testing correction. For two phenotypes and two colonies' comparison, two-way ANOVA analysis was used, followed by Šídák's multiple comparison's test. n=2-3 independent biological replicates were used for all experiments. n.s. denotes a non-significant difference. *p<0.05, **p<0.01, ***p<0.001, ****p<0.0001.

### Gene Overlap Analysis AD Brain Single-Cell RNA-Sequencing

Differential gene expression analysis was performed using RNA-sequencing of AT8− and AT8+ (NFT-bearing) excitatory neurons from a published human AD brain dataset (Otero-Garcia et al., 2022) and the FindMarkers function and MAST in Seurat. Differential gene expression overlap analysis was performed using the 4R-P301S+K18 vs 4R+K18 DEGs from the bulk RNA-seq analysis, 4R-P301S+K18 vs 4R-P301S DEGs from the pseudo bulk scRNA-seq analysis, and AT8+ vs AT8− DEGs for the AD brain analysis. Gene overlap statistical significance was assessed using the GeneOverlap R package (Shen, 2022).

### Calcium Imaging Analysis

For every recorded time-lapse image, ROIs were selected covering all identifiable cell bodies using a semi-automated algorithm in NIS-Elements AR (Nikon). Neurons were categorized based on HaloTag signal punctate structures corresponding to large Tau inclusions (−puncta=without inclusion, +puncta=with inclusion). Further quantifications were performed using custom-written MATLAB (Mathworks) scripts. For single-cell activity, the fluorescence time course was measured by averaging all pixels within individual ROIs. Then the CaPTure toolbox was used to extract spike activity by a rolling average method called ‘DFF’ (Jia et al., 2011; Tippani et al., 2022). A percentile-based threshold (mean+2 standard deviations of fluorescence) was employed to detect peak events. The amplitude and frequency of each ROI was analyzed as the single-neuron activity. To determine the network activity, the synchronous firing rate of the entire cell population in the FOV was measured (Sun and Südhof, 2021). For KCl stimulation experiments, each image frame was divided by an average of all frames acquired during the first 0.5-sec window, which functioned as a baseline. Photobleaching was corrected by fitting exponentially weighted moving averages (Jia et al., 2011)

### Primary Screen Analysis

The primary screen was analyzed using the published MAGeCK-iNC bioinformatics pipeline (Tian et al., 2019). Briefly, raw sequencing reads from next-generation sequencing were cropped and aligned to the reference using Bowtie v.0.12.9 to determine sgRNA counts in each sample. The quality of each screen was assessed by plotting the log 10 (counts) per sgRNA on a rank-order plot. Raw phenotype scores and significance p-values were calculated for target genes, as well as for ‘negative-control-quasi-genes’ that were generated by random sampling with replacement of five non-targeting control (NTC) sgRNAs from all NTC sgRNAs. The final phenotype score for each gene was calculated by subtracting the raw phenotype score by the median raw phenotype score of ‘negative-control-quasi-genes’ and then dividing by the standard deviation of raw phenotype scores of ‘negative-control-quasi-genes’. The hit strength, defined as the product of knockdown phenotype score and −log 10(p-value), was then calculated for all genes in the library and for ‘negative-control-quasi-genes’ generated above. Hit genes were determined based on the hit strength cutoff corresponding to a false-discovery rate (FDR) of 0.1. To find enriched annotations within hit genes, Gene Set Enrichment Analysis was performed for MC1− and MC1+ neurons using the Molecular Signatures Database C5 ontology gene sets (Liberzon et al., 2011; Subramanian et al., 2005).

### Usenamine A Preparation

### Usenamine A: (2E,9bR)-6-acetyl-2-(1-aminoethylidene)-7,9-dihydroxy-8,9b-dimethyldibenzouran-1,3-dione

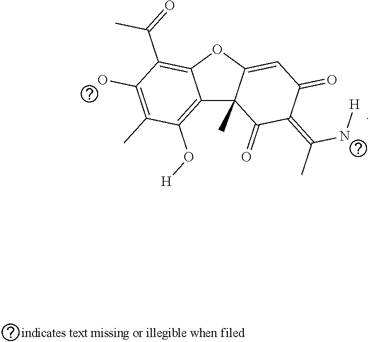 [SMILES: CC(=O)c1c(O)c(C)c(O)c2c1OC1=CC(=O)/C(=C(/C)N)C(=O)[C@@]12C]

Usenamine A was prepared using the commercially available (+)-usnic acid (Combi Block) in one step as described in the literature1. Briefly, (+) usnic acid (900 mg, 2.61 mmol) in absolute ethanol (9 ml) and concentrated ammonium hydroxide (28-30% in water, w/v) under nitrogen was heated with stirring at 80° C. for 2 h. The resulting yellow solution was concentrated to ⅓rd of the original volume and extracted using ethyl acetate (3×). The combined organic layers were washed with water and brine, dried over anhydrous sodium sulfate and concentrated to dryness under reduced pressure. The resulting residues were purified using a short bead of Silica gel to yield Usenamine A (636 mg, 71%), the identity of which was confirmed using MS (342.23, M⁺) and ¹H NMR data found identical to the reported data (Bruno et al. 2013; Mok et al. 2018).

Differentiation of Neurons from 4R-P301S iPSCs.

Pre-differentiation of human iPSCs into neurons was initiated by plating 1.5×10⁶ iPSCs in one well Matrigel-coated 6-well plates with Knockout DMEM/F-12 media containing doxycycline (2 pg/mL), N2 supplement, non-essential amino acids, brain-derived neurotrophic factor (10 ng/mL, Peprotech), neurotrophin-3 (10 ng/mL, PeproTech) and ROCK inhibitor (Y-27632, Cayman chemicals). The media was replaced the next day without ROCK inhibitor, and pre-differentiation was maintained for a total of three days. On day 0, the pre-differentiated precursor cells were dissociated with Accutase and re-plated generally onto Laminin-coated coverslips (Neuvitro) at 1.5×10⁵ cells/coverslip or 12-well tissue culture plates (Corning) at 3.8×10⁵ cells/well for the growth of neuronal cultures in Neurobasal Plus media containing B27 supplement, Glutamax, BDNF (10 ng/mL) and NT3 (10 ng/mL) with doxycycline (2 μg/mL). Half of the medium was replaced on day 3, as well as on day 7 with the removal of doxycycline from the fresh media. The medium volume was doubled on day 21. Thereafter, one-half of the medium was replaced with fresh medium weekly until the cells were collected. All human iPS cell lines used in this work have been regularly tested for mycoplasma (Lonza) and karyotyped (MSK Molecular Cytogenetics Core).

### Preparation and Seeding of Recombinant K18-P301L Tau Fibrils

Myc-tagged K18-P301L Tau was expressed and purified as previously described2. Briefly, protein expression was induced in Terrific Broth containing the chemical chaperone betaine (10 mM) and IPTG (500 uM) for 3 hrs at 30° C. Tau was purified via the following major steps: mechanical lysis, boiling, centrifugation, and cation exchange. Purified Tau fractions were dialyzed into aggregation assay buffer (PBS pH 7.4, 2 mM DTT). To minimize potential endotoxin contamination, purified tau was incubated with poly(epsilon-lysine) conjugated resin (Pierce), then tested post-treatment for endotoxin levels using the ToxinSensor™ Chromogenic LAL Endotoxin Assay Kit (Genscript). Endotoxin levels of tau were <0.1 EU/mL at working concentrations. Purified tau aliquots were stored at −80° C. prior to aggregation. To induce tau aggregation, 88 μg/mL of freshly prepared heparin sodium salt (Santa Cruz Biotechnology) was added to K18-P301L Tau (20 uM) in aggregation assay buffer. Aggregation was carried out in low-retention 1.7 mL microcentrifuge tubes at 37° C. with shaking at 800 rpm for 24 hr. Aggregated tau was isolated by centrifugation at 100,000×g for 1 hr at 4° C. Pelleted tau aggregates were resuspended in PBS (pH 7.4) with sterile plastic pestles and stored in low-binding tubes (CoStar) at −80° C. Tau fibril preparations were retested to confirm endotoxin levels <0.1 EU/mL at working concentrations. The concentration of aggregated tau was quantified by Pierce BCA assay. Fibrils were thawed on ice and mixed by pipetting, and the volume needed for seeding was transferred to 100 uL sterile DPBS. The fibrils were sonicated at 4° C., 10 minutes on/off, 30-second pulse, and amplitude of 40% using a water bath sonicator (EpiSonic 2000, EpigenTek). The fibrils were added to appropriate media volume in wells with neurons to achieve a 3 μg/ml final concentration.

### UBA5 Inhibitor Treatment and Flow Cytometry

4R-P301S iPSCs were seeded at the density of 1.2×10⁷ per plate in 10-cm plates in pre-differentiation medium. On day 0, pre-differentiated neurons were replated at the density of 4×10⁵ per well in poly-D-lysine (PDL) pre-coated 12-well plates (Corning) in maturation medium. On day 5, iPSCs differentiated neurons were treated with three UBA5 inhibitors separately ay various concentrations. On day 7, iPSCs differentiated neurons were treated with 3.0 pg/mL K18 fibrils for 2 weeks. On day 21, neurons in 12-well plates were collected for flow cytometry analysis.

For flow cytometry, briefly, human iPSC-differentiated neurons were dissociated from the plate with Papain solution (20U/mL Papain and 5 mM MgCl2 in HBSS) at 37° C. for 30 mins. Papain was quenched with 3× volume DMEM with 10% FBS. Cells were fixed with zinc fixation buffer (0.1M Tris-HCl with pH=6.5, 0.5% ZnCl₂, 0.5% Zn Acetate and 0.05% CaCl₂) overnight at 4° C. The next day, samples were washed twice with TBS and resuspended in permeabilization buffer (10% Donkey Serum, 10% 10×TBS, 3% BSA, 1% glycine, 0.5% Tween-20) for 15 mins. Primary antibodies are added into permeabilization buffer by pipetting up and down to separate cells into a single cell suspension, and samples are incubated either at 4° C. overnight. The second day, the samples were washed twice with TBS and incubated in permeabilization buffer with secondary antibodies at room temperature for 1 hr. Samples were washed twice with TBS, analyzed with Attune NxT Flow Cytometer (Thermo Fisher), and the data were processed using FlowJo v10 software. To detect endogenous tau inclusions, anti-MC1 (1:200, Peter Davies) primary antibody was used.

| | | |
|---|---|---|
| REAGENT or RESOURCE | SOURCE | IDENTIFIER |

Antibodies

| | | |
|---|---|---|
| Rabbit anti-NANOG | Abcam | Cat#ab21624; RRID: AB_446437 |
| Rabbit anti-OCT4 | Abcam | Cat#ab19857; RRID: AB_445175 |
| Rabbit anti-SOX2 (D6D9) | Cell Signaling | Cat#3579; RRID: AB_2195767 |
| Mouse anti-TRA-1-60 | Abcam | Cat#ab16288; RRID: AB_778563 |
| Mouse anti-TRA-1-81 | Abcam | Cat#ab16289; RRID: AB_2165986 |
| Mouse anti-SSEA4 | Abcam | Cat#ab16287; RRID: AB_778073 |
| Rabbit anti-MAP2 | Millipore Sigma | Cat#AB5622 |
| Chicken anti-MAP2 | Novus Biologicals | Cat#NB300-213; RRID: AB_2138178 |
| Rabbit anti-GAPDH | GeneTex | Cat#GTX100118; RRID: AB_1080976 |
| Mouse anti-Tau 3-repeat isoform (RD3) | Sigma-Aldrich | Cat#05-803; RRID: AB_310013 |
| Mouse anti-Tau 4-repeat isoform (ET3) | Sigma-Aldrich | Cat#05-804; RRID: AB_310014 |
| Mouse anti-p Tau (AT8) | Thermo Fisher Scientific | Cat#MN1020; RRID: AB_223647 |
| Mouse anti-p-PHF-Tau (AT180) | Thermo Fisher Scientific | Cat#MN1040; RRID: AB_223649 |
| Mouse anti-p-PHF-Tau (AT270) | Thermo Fisher Scientific | Cat#MN1050; RRID: AB_223651 |
| Mouse anti-total Tau (HT7) | Thermo Fisher Scientific | Cat#MN1000; RRID: AB_2314654 |
| Rabbit anti-oligomeric Tau (TTC-18) | Rakez Kayed, The University of Texas | Gift |
| | Medical Branch at Galveston | |
| Mouse anti-conformationally abnormal Tau (MC1) | Peter Davies, Albert Einstein College | Cat#MC1; RRID: AB_2314773 |
| | of Medicine | |
| Rabbit anti-UFM1 | Abcam | Cat#ab109305; RRID: AB_10864675 |
| Rabbit anti-GFP | Abcam | Cat#ab6556; RRID: AB_305564 |
| Human FC Block | BD Biosciences | Cat #564220; RRID: AB_2869554 |
| PE/Cy7 anti-human CD71 (TFRC) | BioLegend | Cat #334112; RRID: AB_2563119 |
| Rabbit anti-TFRC | Abcam | Cat#ab84036 |
| Mouse anti-tubulin β 3 (TUBB3) | BioLegend | Cat#801201 |

Biological samples

| | | |
|---|---|---|
| Human brain tissue | University of Pennsylvania brain bank | N/A |

Chemicals, peptides, and recombinant proteins

| | | |
|---|---|---|
| mTeSR Plus kit | Stem Cell Technologies | Cat#05825 |
| ReLeSR | Stem Cell Technologies | Cat#05872 |
| Y-27632 | Stem Cell Technologies | Cat#72308 |
| Matrigel hESC-Qualified Matrix | Corning | Cat#354277 |
| Matrigel GFR Basement Membrane Matrix | Corning | Cat#356231 |
| Doxycycline hyclate (Dox) | Sigma-Aldrich | Cat#D9891 |
| Brain-derived neurotrophic factor (BDNF) | PeproTech | Cat#450-02 |
| Neurotrophin-3 (NT-3) | PeproTech | Cat#450-03 |
| Cultrex 3-D Culture Matrix Laminin I | Trevigen | Cat#3446-005-01 |
| KnockOut DMEM/F-12 | Thermo Fisher Scientific | Cat#12660012 |
| Neurobasal Plus Medium | Thermo Fisher Scientific | Cat#A3582901 |
| DMEM/F-12 | Thermo Fisher Scientific | Cat#11320033 |
| GlutaMAX Supplement | Thermo Fisher Scientific | Cat#35050061 |
| MEM Non-Essential Amino Acids Solution (100X) | Thermo Fisher Scientific | Cat#11140050 |
| StemPro Accutase | Thermo Fisher Scientific | Cat#A1110501 |
| N-2 Supplement | Thermo Fisher Scientific | Cat#17502048 |
| B-27 Plus Supplement | Thermo Fisher Scientific | Cat#A35828-01 |
| DPBS, no calcium, no magnesium | Thermo Fisher Scientific | Cat#14190144 |
| DPBS, with calcium and magnesium | Coming | Cat#21-030-CM |
| PBS, pH 7.4 | Thermo Fisher Scientific | Cat#10010023 |
| Protease Inhibitor Cocktail | Millipore Sigma | Cat#P8340 |
| Phosphatase Inhibitor Cocktail 2 | Millipore Sigma | Cat#P5726 |
| Phosphatase Inhibitor Cocktail 3 | Millipore Sigma | Cat#P0044 |
| Trichostatin A | Millipore Sigma | Cat#T8552 |
| Nicotinamide | Millipore Sigma | Cat#72340 |
| Tau Protein Ladder, 6 isoforms human | Millipore Sigma | Cat#T7951 |
| Lambda Protein Phosphatase | NewEnglandBiolabs | Cat#P0753S |
| Nitrocellulose membrane | Biorad | Cat#1620115 |
| Hanks' Balanced Salt Solution (HBSS) | Sigma-Aldrich | Cat#H9394 |
| Fetal Bovine Serum | Thermo Fisher Scientific | Cat#10439001 |
| Trypsin-EDTA (0.05%), phenol red | Thermo Fisher Scientific | Cat#25300054 |
| Opti-MEM I Reduced Serum Medium | Thermo Fisher Scientific | Cat#31985070 |
| Alt-R ™ S.p. HiFi Cas9 Nuclease V3 | IDT | Cat#1081060 |
| Papain, Lyophilized | Worthington Biochemical | Cat#LS003118 |
| TransIT-Lenti Transfection Reagent | Mirus Bio | Cat#MIR6600 |
| Lipofectamine 3000 | Thermo Fisher Scientific | Cat#L3000015 |
| Lenti-X Concentrator | Takara | Cat#631232 |
| Lentivirus Precipitation Solution | Alstem | Cat#VC125 |
| Puromycin | Thermo Fisher Scientific | Cat#A1113803 |
| Bovine Serum Albumin | Sigma Aldrich | Cat#A7906-100G |
| Janelia Fluor 549 analog JFX-549 | HHMI Janelia | Gift |
| Clozapine N-oxide | Tocris | Cat#4936 |
| NucRed ™ Live 647 ReadyProbes ™ Reagent | Thermo Fisher Scientific | Cat#R37106 |
| iScript ™ Reverse Transcription Supermix | Bio-Rad | Cat#1708840 |
| Real-time PCR was performed using SsoAdvanced | Bio-Rad | Cat#1725270 |
| Universal SYBR ® Green Supermix | | |

Critical commercial assays

| | | |
|---|---|---|
| P3 Primary Cell 4D- Nucleofector ™ X Kit S | Lonza | Cat#V4XP-303 |
| Pierce BCA Protein Assay Kit | Thermo Fisher Scientific | Cat#23225 |
| QuickRNA MicroPrep Kit | Zymo | Cat#R1051 |
| Illumina Stranded mRNA Prep | Illumina | Cat#20040532 |
| Human Tau (Total) ELISA Kit | Thermo Fisher Scientific | Cat#KHB0042 |
| Qiagen Plasmid Maxi kit | Qiagen | Cat#12963 |
| Laminin coated coverslips | Neuvitro Corporation | Cat#GG-12-15-Laminin |
| Poly-D-Lysine 150 mm TC-treated Gridded Culture | Corning | Cat#354550 |
| Dishes | | |
| ToxinSensor Chromogenic LAL Endotoxin Assay Kit | GenScript | Cat#L00350C |
| VECTASHIELD ® Antifade Mounting Medium without | Vector Laboratories | Cat#H-1000-10 |
| DAPI | | |
| VECTASHIELD ® Antifade Mounting Medium with | Vector Laboratories | Cat#H-1200-10 |
| DAPI | | |

Deposited data

| | |
|---|---|
| Immunoblot data | This paper |
| Raw and processed data (bulk RNA-sequencing) | This paper |

Experimental Models: Cell Lines

| | | |
|---|---|---|
| HEK293T | ATCC | Cat#CRL-11268 |
| Human WTC11 iPSC with Ngn2 transgene integration | Wang et al., 2017 | N/A |

Oligonucleotides

| | | |
|---|---|---|
| Primers for sequencing | This paper | N/A |
| Primers for qPCR | This paper | N/A |

Recombinant DNA

| | | |
|---|---|---|
| psPAX | Didier Trono | Addgene #12260 |
| pMD2.G | Didier Trono | Addgene #12259 |
| pMDLg/pRRE | Didier Trono | Addgene #12251 |
| pRSV-REV | Didier Trono | Addgene #12253 |
| pC13N-CLYBL-CAG-dCas9-BFP-KRAB | Martin Kampmann | Addgene #127968 |
| pMK1334 | Martin Kampmann | Addgene #127965 |
| pFUGW-hSyn-jGCaMP8f | This paper | N/A |
| PFUGW-hSyn-hM4D(Gi)-mCherry | This paper | N/A |
| pFUGW-eGFP | Manu Sharma | Gift |
| pFUGW-GFP-VAMP72-120 (VAMPDN) | Manu Sharma | Gift |

Software and Algorithms

| | | |
|---|---|---|
| R 4.2.2 | The R project | https://www.r-project.org/ |
| RStudio 2022.07.2 | RStudio: Integrated Development for | https://rstudio.com |
| | R. RStudio | |
| GSEA | Mootha et al., 2003 | https://www.gsea-msigdb.org/gsca/msigdb/index.jsp |
| Adobe Illustrator | Illustrator v26.5.2 | https://www.adobe.com/products/illustrator.html |
| GraphPad Prism 6 | Prism v9.2 | https://www.graphpad.com |
| FlowJo | FlowJo v10 | https://www.flowjo.com/ |
| NIS Elements AR 5.21.03 64-bit | Nikon Instruments Inc. | https://www.microscope.healthcare.nikon.com/ |
| | | products/software/nis-elements |
| Bowtie- 0.12.9 | Langmead et al., 2009 | https://bowtie-bio.sourceforge.net/index.shtml |
| MAGeCK- 0.5.7 | Li et al., 2014 | https://sourceforge.net/p/mageck/wiki/Home/ |
| MAGeCK-iNC | Tian et al., 2019 | https://kampmannlab.ucsf.edu/mageck-inc |
| MATLAB | MATLAB R2022a | https://www.mathworks.com/products/matlab.html |
| Seurat | Seurat v4.3 | https://satijalab.org/seurat/ |
| Fiji/ImageJ | Fiji v2.8 | RRID: SCR_002285 |

### Results

Generation and Characterization of 4R-Tau and Human iPSC-Derived Neurons

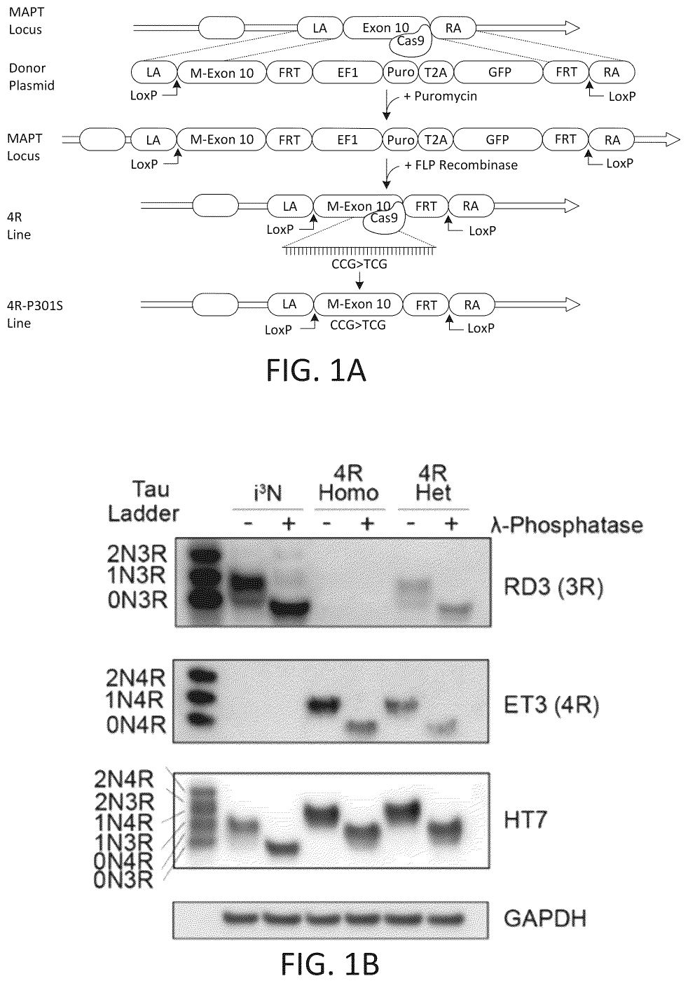

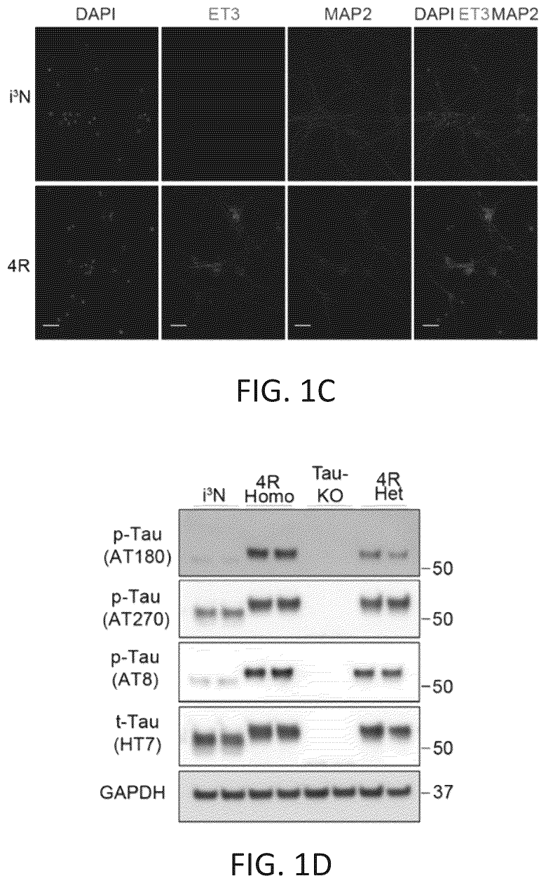

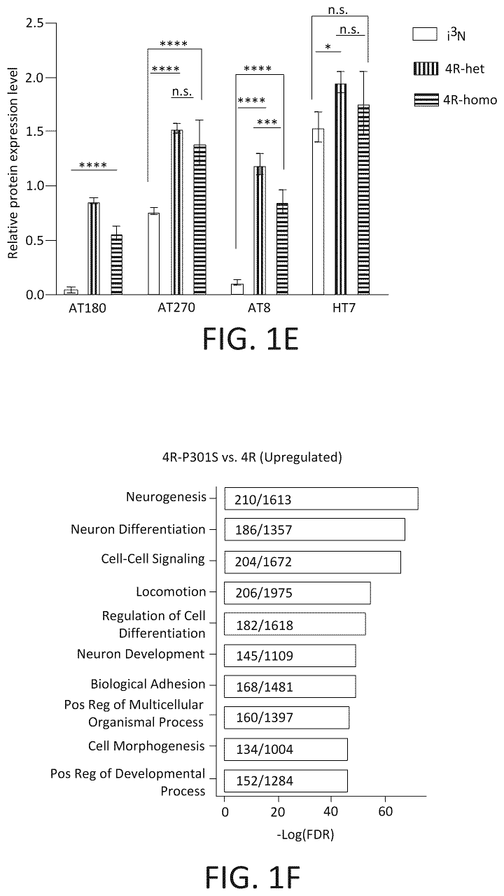

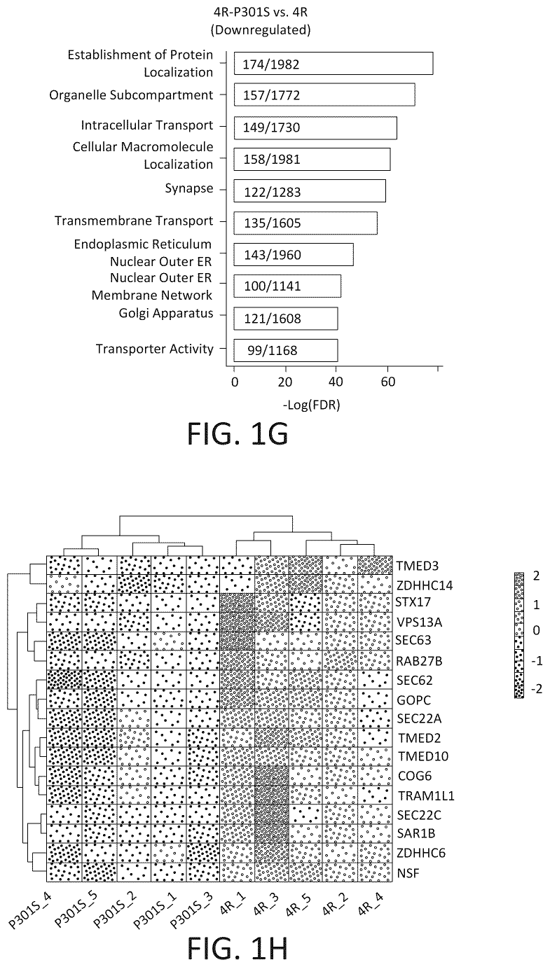

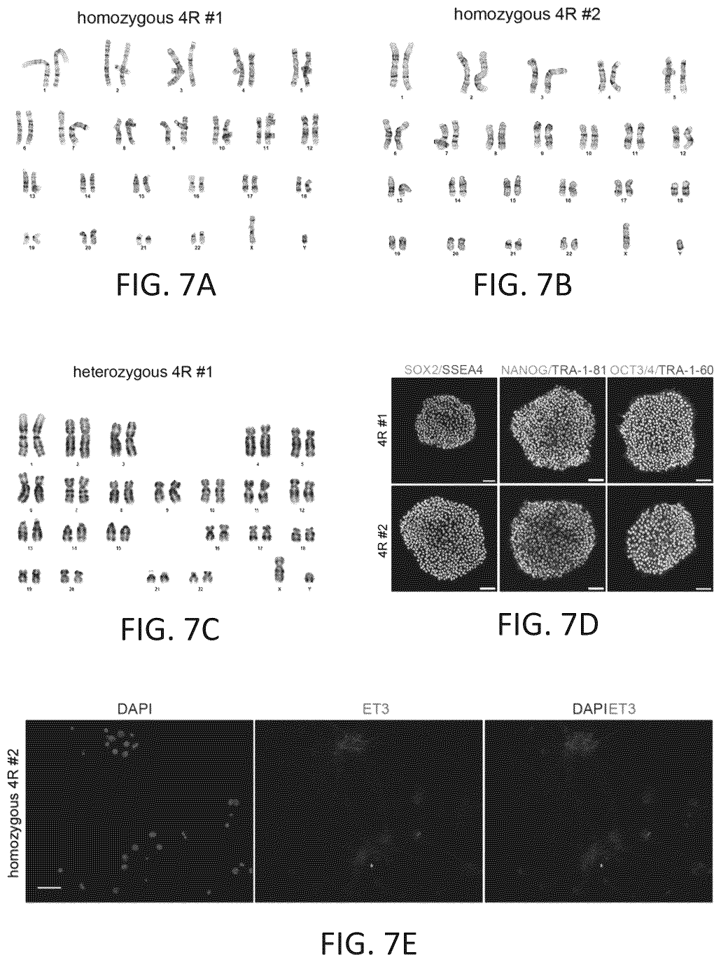

Among the six isoforms of MAPT, the 4-repeat (4R) Tau, resulting from the inclusion of exon 10 via alternative splicing, expresses four microtubule-binding domains (MTBD) and plays roles in the pathogenesis of tauopathies. However, modeling 4R tauopathy in iPSC neurons has been difficult since human iPSC-derived neurons, including i3Neurons that express inducible Neurogenin-2 transcription factor, express very low levels of 4-repeat Tau (Sposito et al., 2015; Verheyen et al., 2015; Wang et al., 2017). To elevate 4R-tau expression, the MAPT locus of i3Neurons was edited via CRISPR/Cas9 using a donor plasmid containing point mutations at the 3′ and 5′ ends of exon 10 that prevent snRNP binding and splicing of the pre-mRNA (FIG. 1A). After puromycin selection and FLP recombinase treatment to excise the GFP marker, clones with one or both alleles of MAPT edited to express 4R-Tau were selected. Two 4R-homozygotic clones (clone #1 and clone #2) and one heterozygotic clone were confirmed with normal karyotypes and markers of pluripotency, including SOX2, SSEA4, NANOG, TRA-1-81, OCT3/4, and TRA-1-60 (FIG. 7A-D).

Homozygotic clone #1 was differentiated into excitatory neurons with the addition of doxycycline, as described (Wang et al., 2017). The parental i3N line and a heterozygotic line with one allele of MAPT locus edited were included as controls. Western blot analyses were performed using antibodies recognizing 3R MTBDs and ET3 specific for exon 10 (Espinoza et al., 2008), and HT7, a pan-tau antibody. Treatment with phosphatase allowed more accurate alignment with the six isoforms of recombinant Tau and served as MW controls (FIG. 1B). Compared to i3N neurons, which express only 3R-tau labeled with RD3 antibody, neurons derived from homozygotic 4R tau (clone #1) express exclusively 4R-tau labeled with ET3 (FIG. 1B). In contrast, neurons derived from a normal karyotype 4R-hetero clone #1 (FIG. 7C) express both 3R-tau and 4R-tau (FIG. 1B). Immunocytochemistry confirmed the immunoreactivity of ET3, a 4R-specific antibody in 4R homozygous clone #1 neurons (FIG. 1C) and clone #2 (FIG. 7E), but not in i3N neurons (FIG. 1C). Interestingly, compared with i3N neurons, both 4R homozygous and heterozygous neurons express higher levels of phospho-tau species, which have been associated with AD and primary tauopathies (FIG. 1D, 1E).

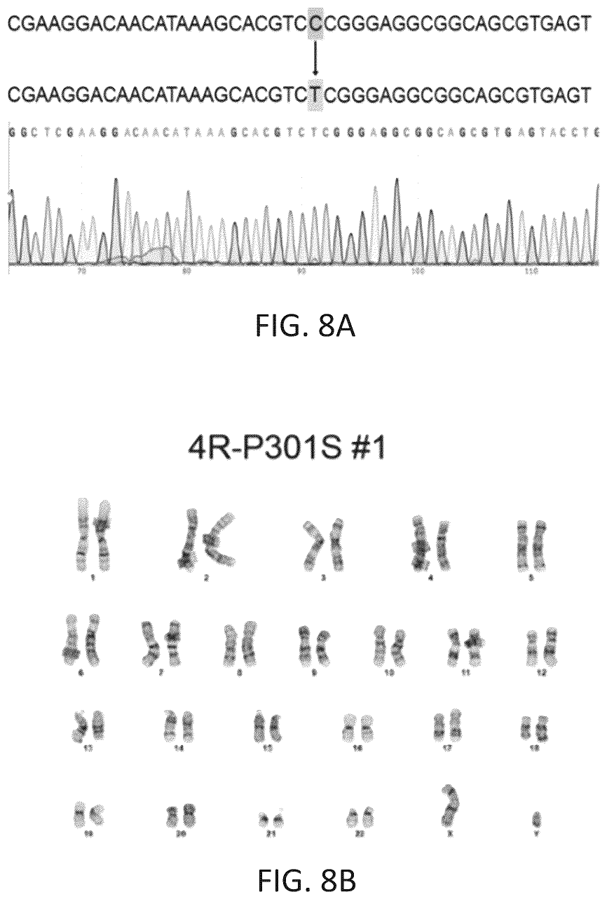

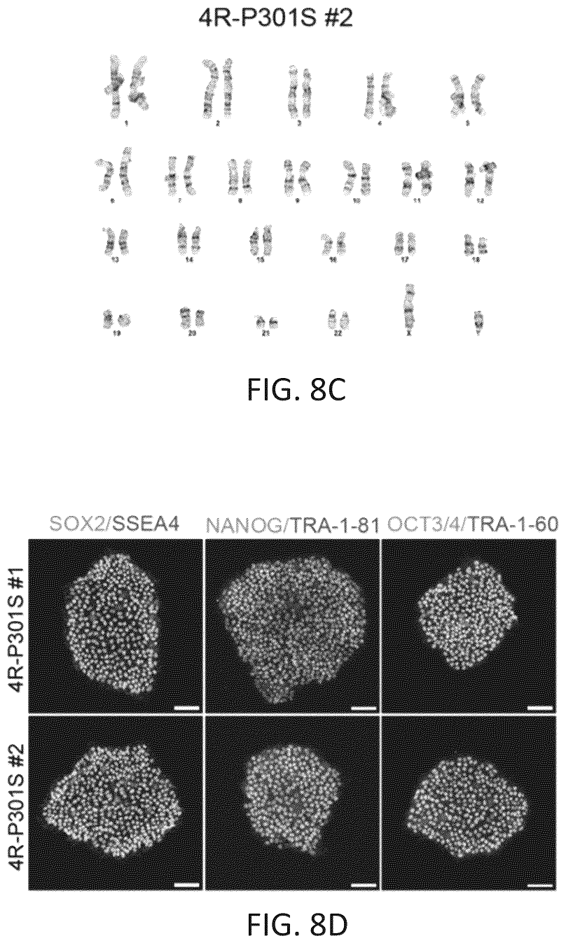

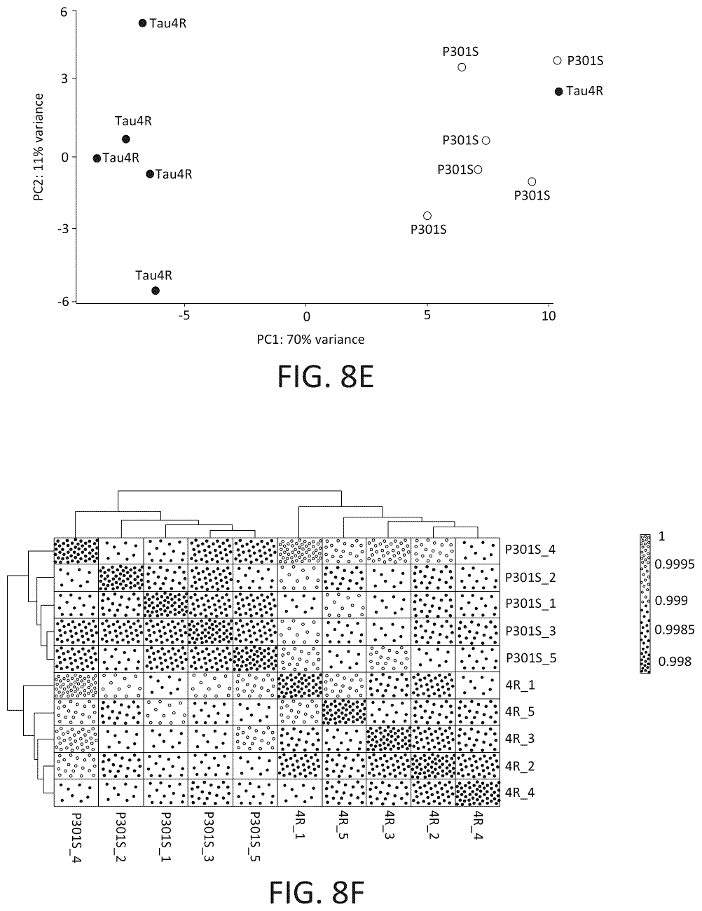

One of the most common frontotemporal dementia (FTD)-linked MAPT mutations, P301S, is located in exon 10 (Yasuda et al., 2005). P301S is also highly aggregation-prone (Allen et al., 2002; Berriman et al., 2003). To model FTD tauopathy, the homozygous 4R-tau (4R) line was edited to include the P301S mutation using a single-stranded DNA oligonucleotide and replacing a proline with a serine at residue 301 (FIG. 1A, FIG. 8A). Two clones of homozygous 4R-P301S-tau-expressing (4R-P301S) iPSCs (FIG. 8B, 8C) were confirmed for normal karyotypes and pluripotency using markers (i.e., SOX2, SSEA4, NANOG, TRA-1-81, OCT3/4, TRA-1-60) (FIG. 8D). To dissect the molecular alterations induced by the P301S mutation, bulk RNA-sequencing analyses of 4R and 4R-P301S neurons were performed (FIG. 8). Unsupervised cluster and principal component analyses (PCA) revealed that 4R neurons and 4R-P301S neurons were clustered separately (FIG. 8E-F), consistent with the pathogenic nature of P301S mutation. Over 2200 genes were altered by P301S mutation. The upregulated genes showed enrichment in Neuron differentiation and Cell-Cell signaling pathways (FIG. 1F), and the downregulated ones were enriched in genes related to Organelle localization, Intracellular transport, and Transmembrane transport (FIG. 1G), some of which were highlighted in the heatmap (FIG. 1H). The P301S mutation downregulated expression of Transmembrane p24 trafficking protein (TMED)3, TMED10, and TMED2 (FIG. 1H), which are all involved in the transport of proteins between the ER and the Golgi apparatus and play roles in maintaining cellular homeostasis and proper folding and transport of proteins in the cell. Other downregulated genes include Syntaxin-17 (STX-17), involved in the fusion of autophagosomes with lysosomes, and Vacuolar Protein Sorting 13 Homolog A (VPS13A), a protein facilitating non-vesicular lipid transfer between organelles, such as the endoplasmic reticulum (ER) and mitochondria, or the ER and endosomes/lysosomes (FIG. 1H). These findings suggest that 4R-P301S neurons are likely to be more vulnerable to proteostasis imbalance.

### Modeling Propagation of 4R-Tau Inclusions

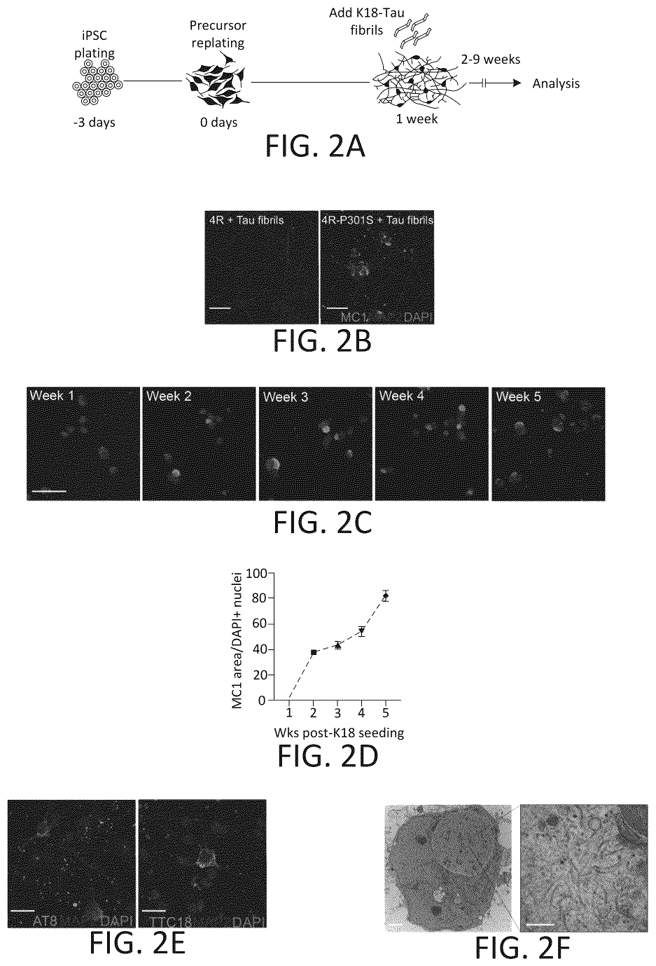

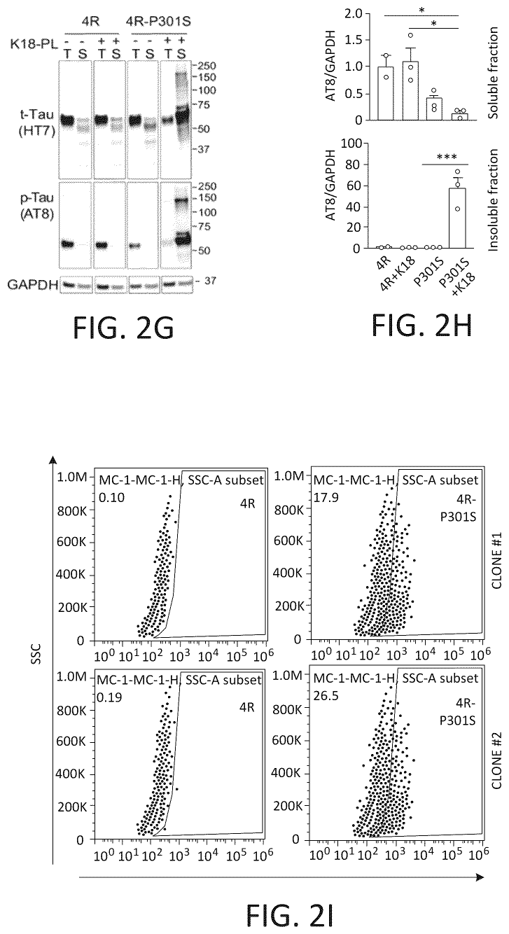

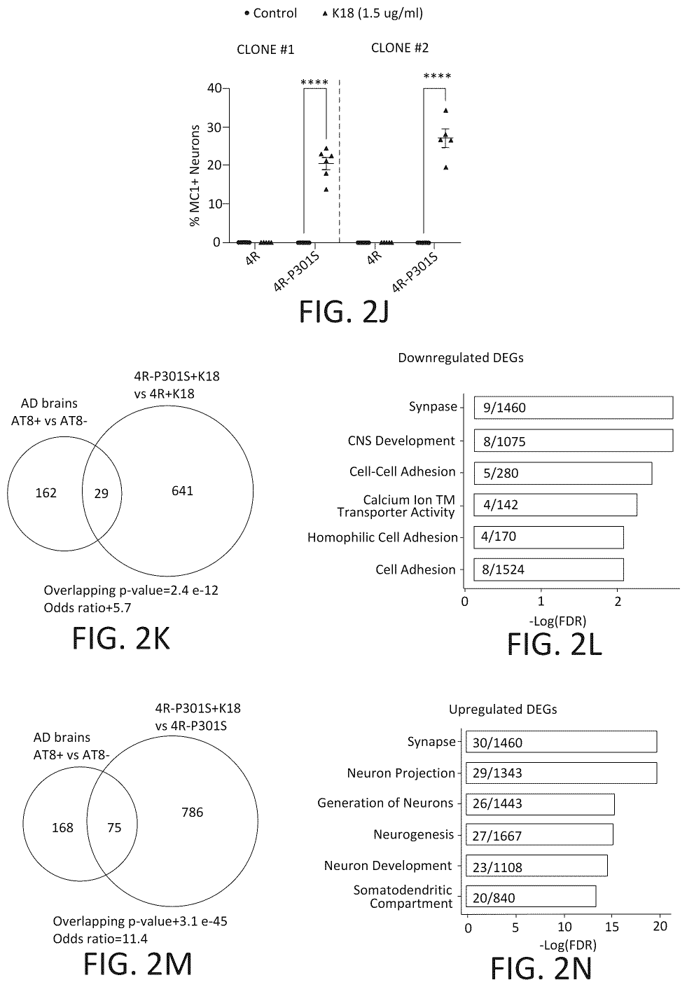

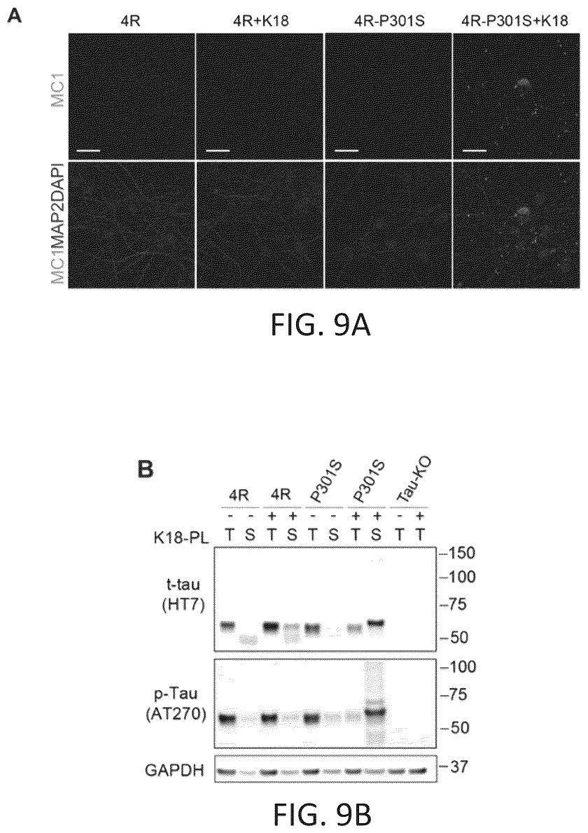

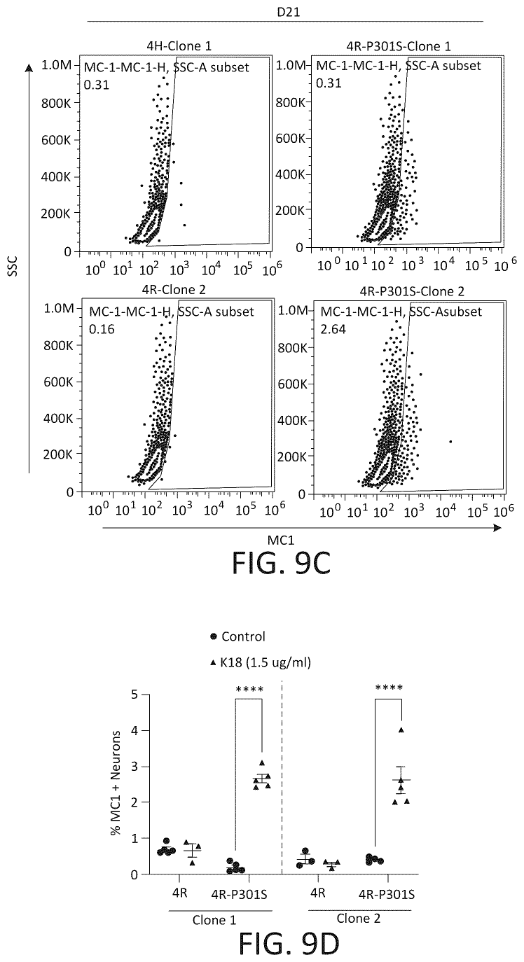

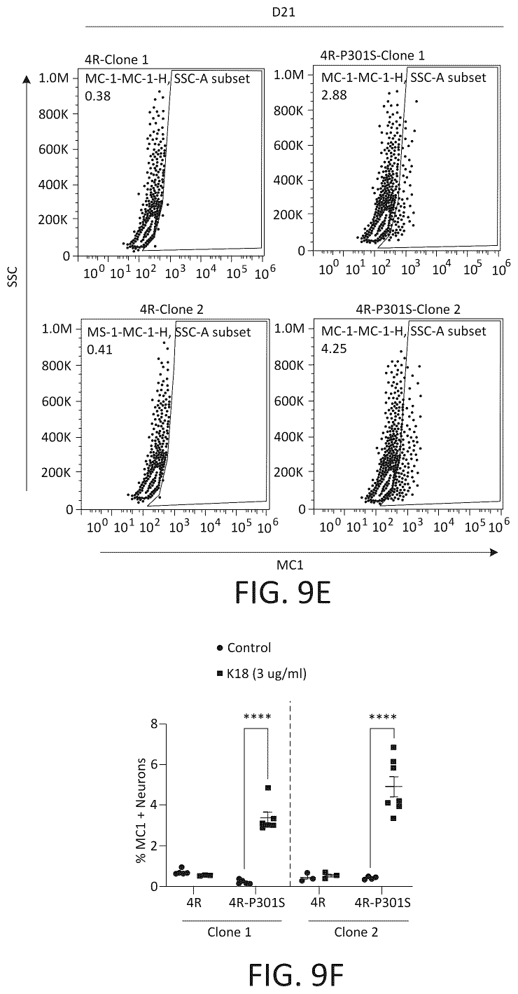

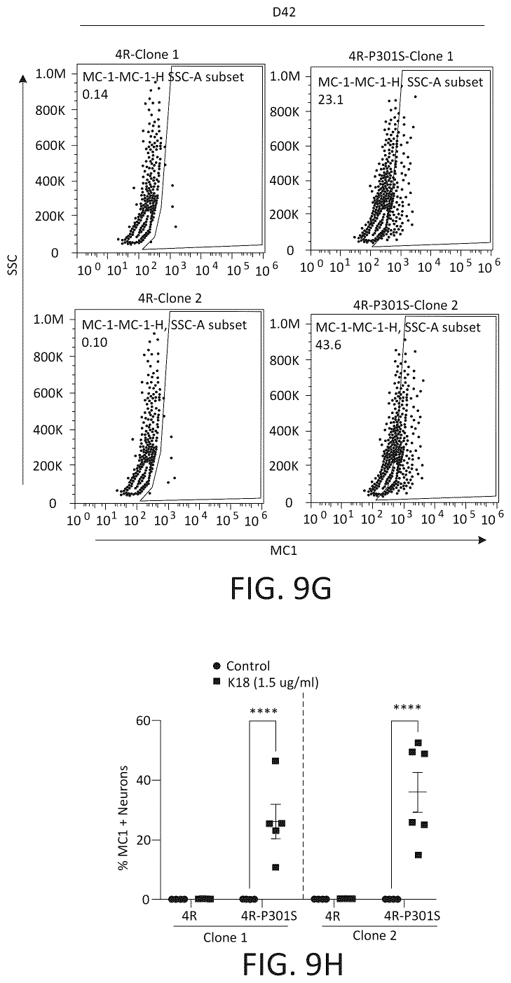

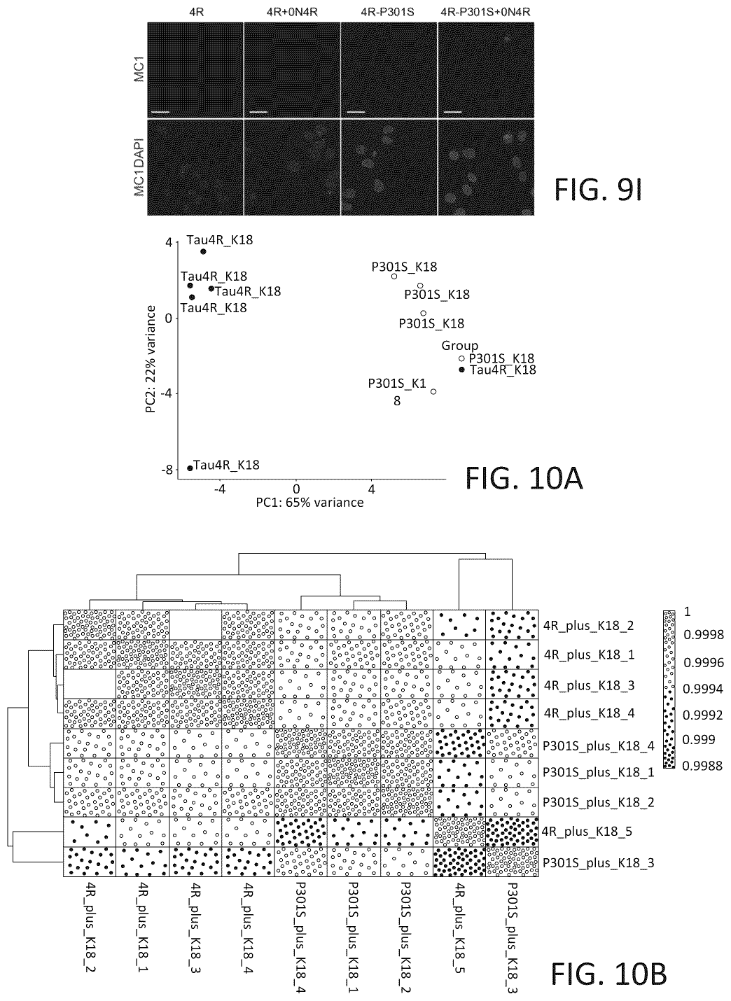

Using the MC1 antibody, a conformation-specific antibody that recognizes disease-specific forms of Tau from human patients (Jicha et al., 1997), no obvious insoluble Tau aggregates were detected in 4R and 4R-P301S neurons even after weeks in culture (FIG. 9A). To model seeding-induced Tau propagation, the 4R or 4R-P301S neurons were treated with Tau fibrils of K18-P301L-tau (K18), a truncated form of human Tau containing only the aggregation-prone repeat domain of the microtubule-binding domain (Gustke et al., 1994)(FIG. 2A). After a 3-5-week treatment, there were robust MC1-positive inclusions only in 4R-P301S, not in 4R neurons (FIG. 2B). Since MC1 immunoreactivity uses domains outside of K18, the MC1+ inclusions are made of endogenous 4R Tau upon seeding with Tau fibrils, a process reflecting templating-induced propagation of misfolded protein. Weekly analyses at 1-5 weeks post-seeding showed a progressive increase in MC1+ inclusions (FIG. 2C, 2D), supporting a prion-like model of Tau propagation. Further analyses revealed that these MC1+ inclusions were immunoreactive to antibodies against oligomers and phosphorylation (FIG. 2E). Transmission electron microscopy (TEM) was performed and detected prominent fibrillar Tau structures in the soma of 4R-P301S neurons seeded with tau (FIG. 2F).

To further analyze the biochemical nature of the inclusions, lysates were first solubilized by Triton-X, and the insoluble fractions were further solubilized by SDS. In 4R neurons, the majority of Tau and phospho-Tau was observed in the Triton-soluble fraction with no high-molecule-weight (MW) Tau and phospho-Tau observed, consistent with the lack of MC1+ aggregates with or without K18 Tau seeding (FIG. 2G). Quantification confirmed that Tau-seeding of 4R-P301S neurons significantly reduced levels of AT8-positive phospho-Tau in the Triton-soluble fraction, while markedly elevating those in the triton-insoluble fraction (FIG. 2H). Another species of phospho-tau (AT270) was also observed in the Triton-insoluble/SDS fraction of 4R-P301S neurons seeded with K18-tau fibrils (FIG. 9B).

Next, flow cytometry with MC1 antibody was used to measure somatic Tau inclusions quantitatively. The extent of tau inclusions at two times, using two independent 4R-P301 S clones to confirm the reproducibility of the phenotype. At Day 21 post-seeding of K18 fibrils (1.5 pg/ml), both clone #1 and clone #2 4R-P301S neurons exhibited highly consistent levels of MC1+ population of neurons (FIG. 9C, 9D). The percentage of MC1+ neurons increased 5-6-fold between Day 21 and Day 42 post-seeding in both clone #1 and clone #2, reflecting seeding-induced propagation/amplification (FIG. 2G, 2H). Increasing the Tau fibrils from 1.5 pg/ml to 3.0 pg/ml resulted in a modest increase in tau inclusions in both 4R-P301S clones, either at Day 21 (FIG. 9E, 9F) or Day 42 (FIG. 9G, 9H) post-seeding. To address whether the mutant tau fibrils induce species-specific templating of mutant tau in 4R-P301S neurons, neurons were seeded with WT 0N4R-tau fibrils for 3 weeks and detected MC1-positive inclusions only in 4R-P301S, not in 4R neurons (FIG. 9I). Thus, Tau aggregation in 4R-P301S neurons is not limited to seeding with fibrils with the same mutated residue.

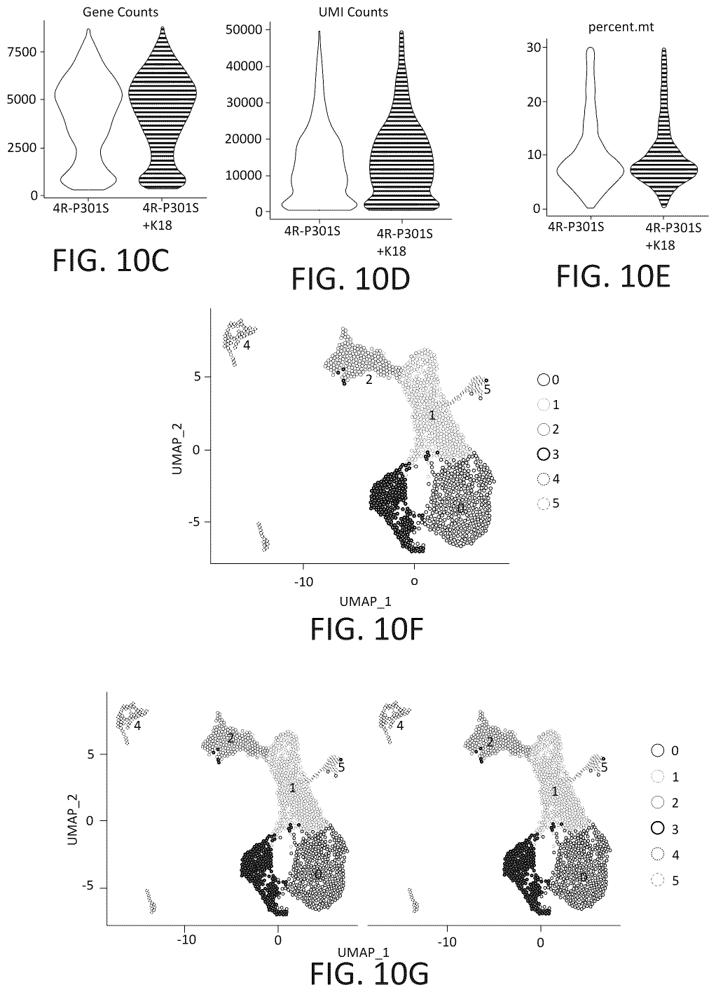

Molecular signatures of tangle-bearing neurons in AD were recently characterized by single-cell RNA sequencing (Otero-Garcia et al., 2022). To interrogate the transcriptomic alterations in MC1+ tau inclusions, bulk RNA-seq was performed and identified the differentially expressed genes (DEGs) in 4R-P301S+K18 that developed seeding-induced inclusions and 4R+K18 neurons that do not, despite the addition of K18 seeds (FIG. 10A, 10B). To assess gene expression similarities between the in vitro platform and post-mortem human AD brains, a gene overlap analysis of the DEGs with those comparing AT8+ vs AT8− excitatory neurons in human AD brains was performed (FIG. 2K). A modest yet significant gene overlap was detected in the downregulated DEGs (odds ratio=5.7, p-value=2.4e-12) with Synapse and Cell-Cell adhesion among the top overlapping pathways (FIG. 2L). To remove possible effects of the P301S mutation, single-cell RNA sequencing was performed to compare the transcriptomes of tau-seeded vs. non-seeded 4R-P301S neurons (FIG. 10C-E). It was presumed the pseudo-bulk analyses of the DEGs would be enriched with molecular signatures of inclusion-containing human neurons. Indeed, comparison with AT8+ vs AT8− excitatory neurons in human AD brains revealed a highly significant overlap in the upregulated DEGs (FIG. 2M) with Synpase and Neuron projection among the top overlapping pathways (odds ratio=11.4, p-value=3.1e-45, FIG. 2N).

### Endolysosomal Dysfunction in 4R-P301S Neurons Promotes Tau Propagation

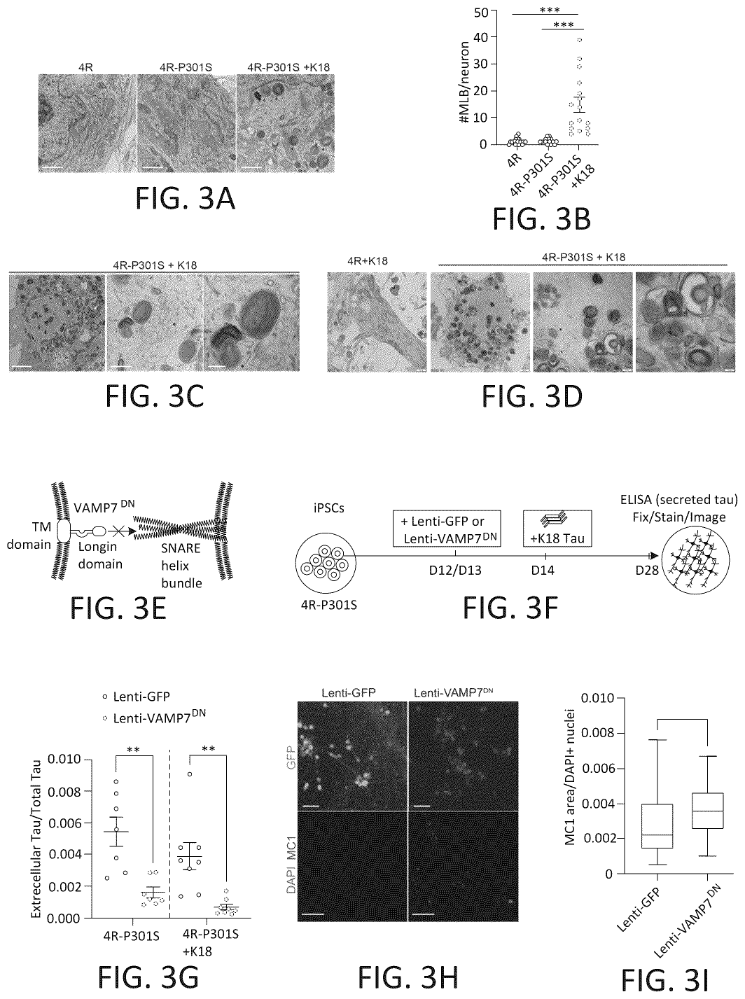

4R-P301S neurons exhibited striking downregulated pathways in Organelle localization, Intracellular transport, and Transmembrane transport (FIG. 1G, 1H). To further dissect alterations of cellular machinery caused by Tau inclusions, TEM analyses were performed to compare subcellular structures in 4R and 4R-P301S neurons with or without K18 seeding. Marked accumulation of abnormal vesicular structures with multilayer membranes was observed in 4R-P301S neurons treated with K18 fibrils, not in 4R neurons or 4R-P301S neurons without seeding (FIG. 3A, 3B). These abnormal structures were reminiscent of multilamellar bodies (MLBs), organelles containing multiple concentric membrane layers of lysosomal origin (Hariri et al., 2000). MLBs were observed in the soma along with Tau inclusions (FIG. 3C), as well as in the processes of 4R-P301 S neurons (FIG. 3D).

The accumulation of MLBs in 4R-P301S neurons with Tau inclusions strongly suggests dysfunctional lysosomal membrane trafficking. Since lysosomal exocytosis has been linked with the spread of misfolded protein aggregates, including alpha-synuclein (Xie et al., 2022), it was reasoned that impaired lysosomal fusion could be involved in the seeding-induced tauopathy model. To block lysosomal fusion, VAMP7 activity was inhibited, a calcium-dependent v-SNARE protein that mediates lysosomal membrane fusion (Arantes and Andrews, 2006), by overexpressing dominant-negative VAMP7 (VAMP7DN) (Xie et al., 2022) (FIG. 3E). 4R-P301 S neurons were infected with Lenti-GFP-VAMP7DN or Lenti-GFP-control, followed by seeding with K18 fibrils, and analyses were performed 2 weeks later (FIG. 3F). Blockage of VAMP7-mediated membrane fusion significantly reduced the amount of extracellular Tau released after KCl-induced depolarization, relative to total Tau, measured with ELISA as described (Tracy et al., 2022) (FIG. 3G). Strikingly, blockage of VAMP7 activity resulted in a significant increase in MC1+ Tau inclusions (FIG. 3H, 3I). Together, these results suggest that impaired membrane fusion, possibly lysosomal exocytosis, exacerbates seeding-induced Tau propagation.

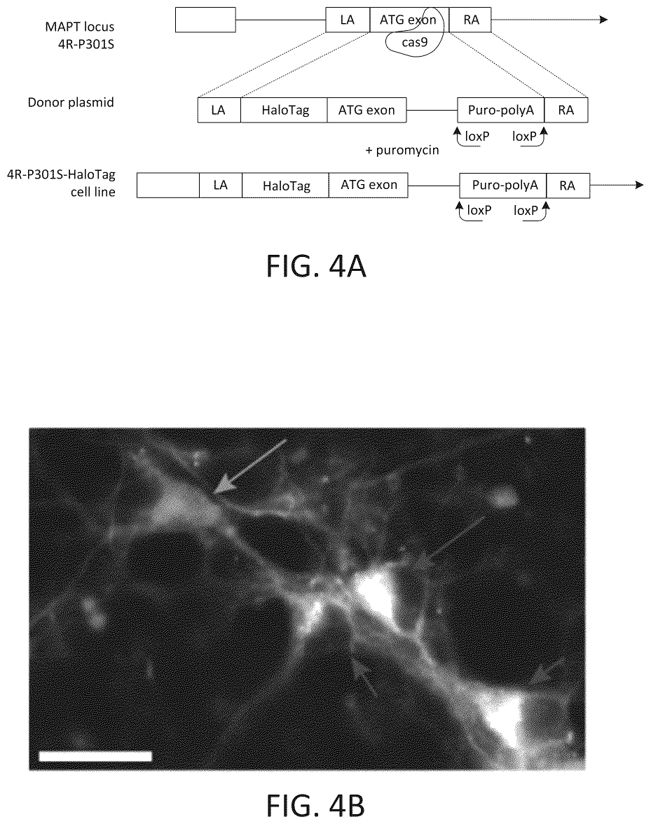

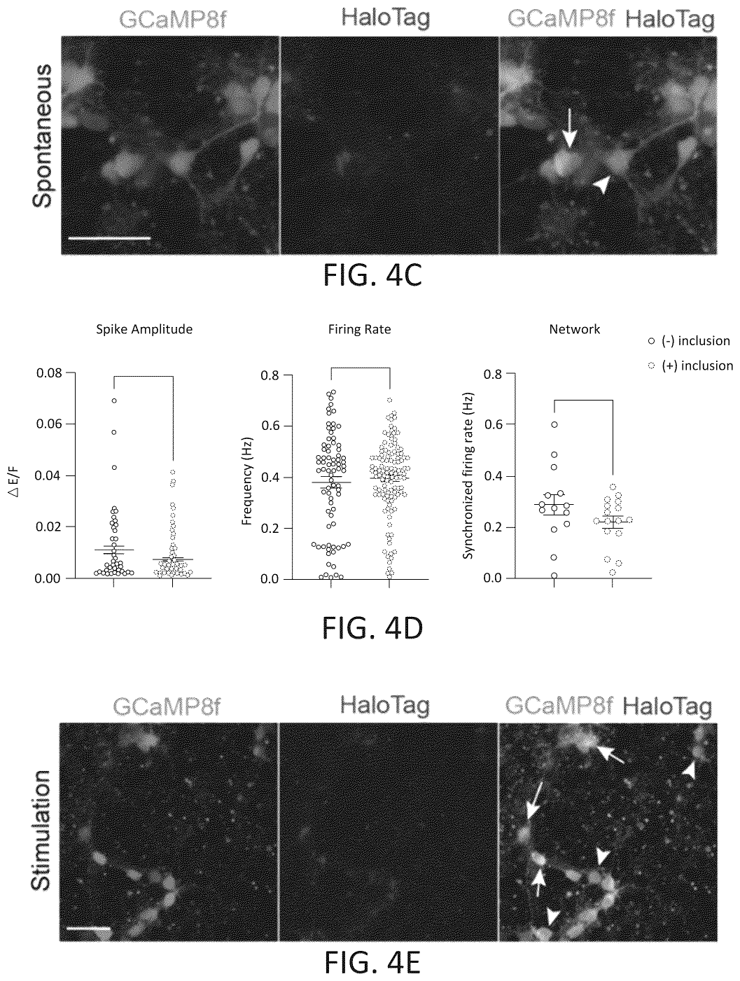

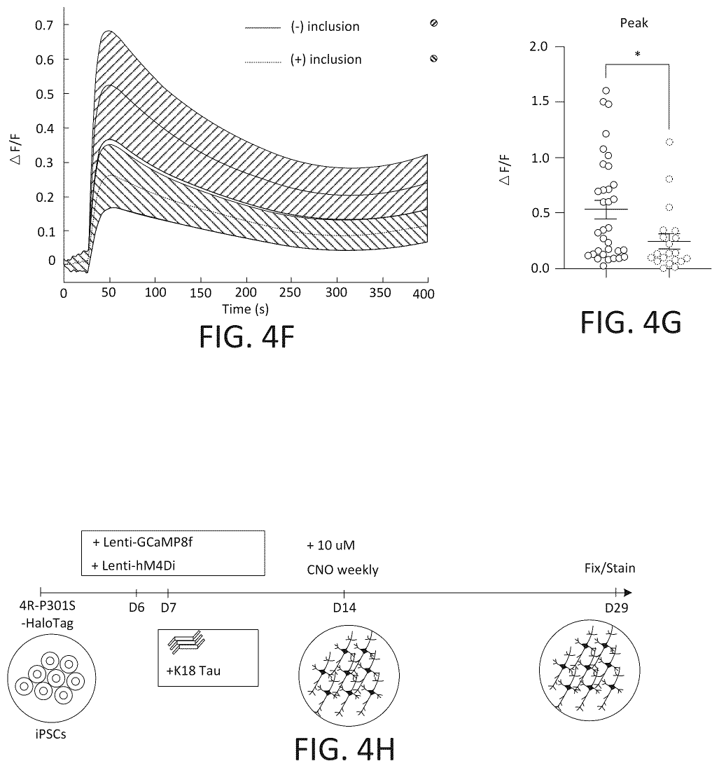

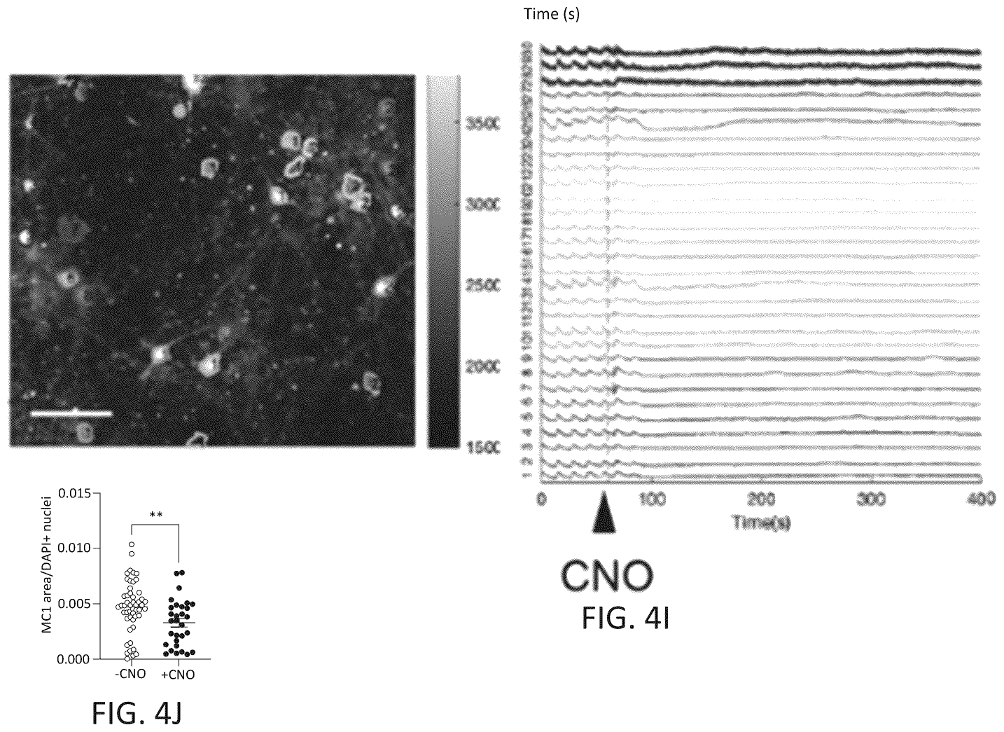

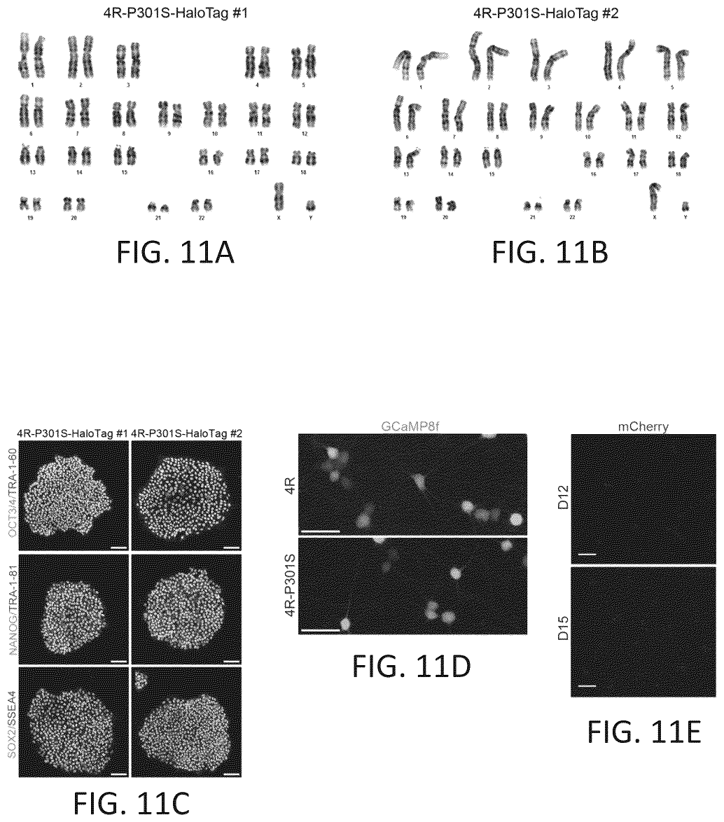

Tau inclusions impair neuronal activity Tau pathology correlates strongly with cognitive decline (Bejanin et al., 2017; Love et al., 2014; Ossenkoppele et al., 2022). However, how neuronal functions are affected by Tau inclusions remains poorly defined due to the challenge of labeling Tau inclusions in live neurons. To track live human neurons with or without Tau aggregates for calcium imaging, the 4R-P301S line was modified to insert a HaloTag at the 5′ end of the MAPT locus using CRISPR-Cas9 gene editing (FIG. 4A). Upon the binding of the HaloTag to a synthetic ligand of choice, HaloTag undergoes a conformational change and fluoresces at a given wavelength, enabling live-cell imaging of endogenous Tau molecules (Grimm et al., 2015). Two independent homozygous 4R-P301S-HaloTag iPSC clones were confirmed for normal karyotypes (FIG. 11A-C), and clone #1 was used for functional experiments. Upon addition of JFX-549 synthetic ligand (Grimm et al., 2015), 4R-P301S neurons with and without obvious aggregated Tau can be visually distinguished at 568 nm (FIG. 4B).

To monitor neuronal activity simultaneously, seeded 4R-P301S neurons were transduced with a new generation of genetically encoded calcium sensor GCaMP8-fast (Lenti-hSynapsin-hGCaMP8f) (Zhang et al., 2023) (FIG. 4C, S5D). Calcium transients, measured by the change in fluorescence over basal fluorescence (dF/F), were recorded over 2 minutes per somal region of interest (ROI) within an image frame (FIG. 4C). ROIs in the soma were then classified into visually distinguishable −inclusion or +inclusion bins, and spontaneous neuronal activities from single neurons were extracted from the calcium trace (FIG. 4C). Compared with neurons lacking inclusions, those with inclusions displayed a significant decrease in spike amplitudes, whereas the firing and synchronicity rates remained unchanged (FIG. 4D). To measure evoke responses, 50 mM KCl was perfused to induce chronic depolarization (FIG. 4E). Neurons with visible somatic inclusions exhibited significant lower trace peak heights than those without visible inclusions (FIG. 4F, 4G). Thus, both spontaneous and stimulation-evoked neuronal activity were impaired in human neurons with Tau inclusions.

### Neuronal Activity Promotes Tau Propagation

The release of Tau is enhanced by neuronal activity in mouse brains and human neurons (Pooler et al., 2013: Tracy et al., 2022; Wu et al., 2016: Yamada et al., 2014). FTD mutations, such as V337M, induce hyperexcitability in human neurons (Sohn et al., 2019). To directly examine the effects of neuronal activity on Tau seeding and spread, chronic silencing of neuronal activity was induced by transducing the viral vector encoding an inhibitory DREADD receptor, hM4Di, genetically modified to respond specifically to the synthetic ligand clozapine-N-oxide (CNO), resulting in suppression of neuronal firing (Zhu and Roth, 2014). 4R-P301 S-HaloTag neurons were infected with mCherry-hM4Di lentivirus, followed by K18 seeding (FIG. 4H). One week after the seeding, CNO was applied to the culture weekly for 2 additional weeks to determine the outcome of chronic suppression of neuronal activity (FIG. 4H). Robust expression of the mCherry-hM4Di receptor was confirmed before and after CNO administration (FIG. 11E). Using calcium imaging, it was confirmed that neuronal activities were silenced upon addition of CNO (FIG. 4I). Levels of Tau inclusions, measured with MC1+aggregates, were significantly reduced by CNO treatment compared with those with vehicle treatment (FIG. 4J). Thus, neuronal activity promotes seeding-induced Tau propagation in human neurons.

### CRISPRi Screen Identifies Genetic Modifiers for Tau Propagation in 4R Tauopathy

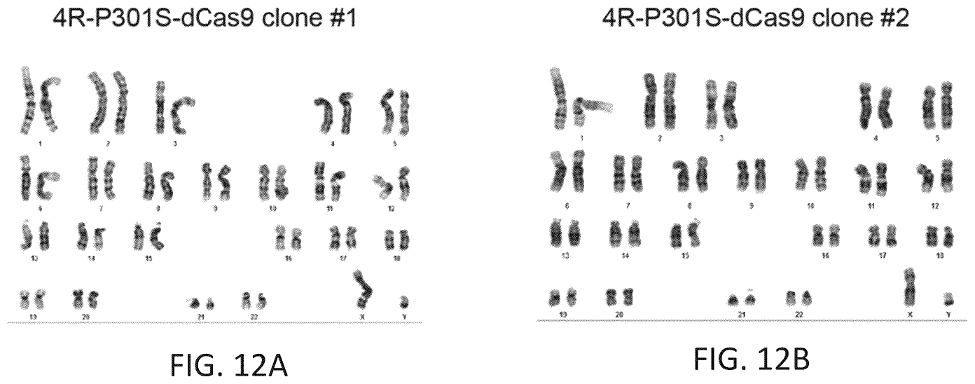

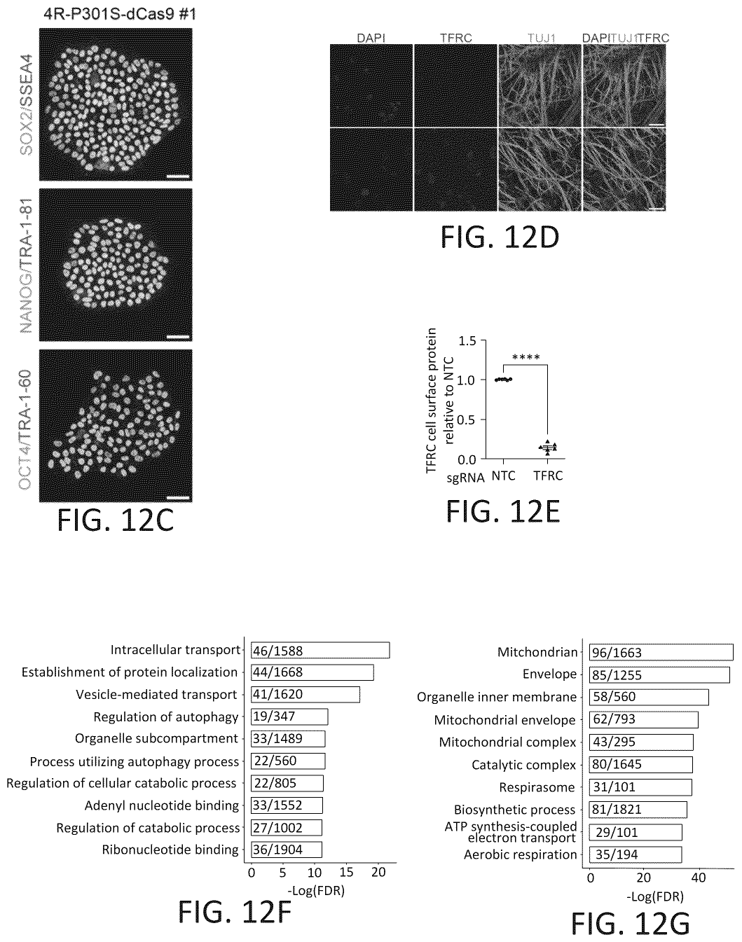

This new model of 4R Tau propagation is engineered from the i3N neuron platform, which facilitates the mass production of iPSC-derived glutamatergic neurons and enables scalable CRISPRi-based functional genomics (Tian et al., 2019). To identify genetic modifiers of Tau propagation, 4R-P301S iPSCs were engineered to stably express dCas9 by inserting CAG promoter-driven dCas9-BFP-KRAB into the CLYBL safe harbor locus by TALENs (Tian et al., 2019). After FACS sorting for BFP and selection with puromycin, two clones of heterozygous 4R-P301S-dCas9 lines were selected and confirmed for normal karyotype, clone #1 (FIG. 12A) and clone #2 (FIG. 12B). 4R-P301S-dCas9 iPSC clone #1 was stained for pluripotency markers OCT4, TRA-1-60, NANOG, TRA-1-81, SOX2, SSEA4 (FIG. 12C) and was used for subsequent CRISPRi experiments. The knockdown efficiency of the 4R-P301S-dCas9 iPSC clone #1 was confirmed by TFRC staining (FIG. 12D).

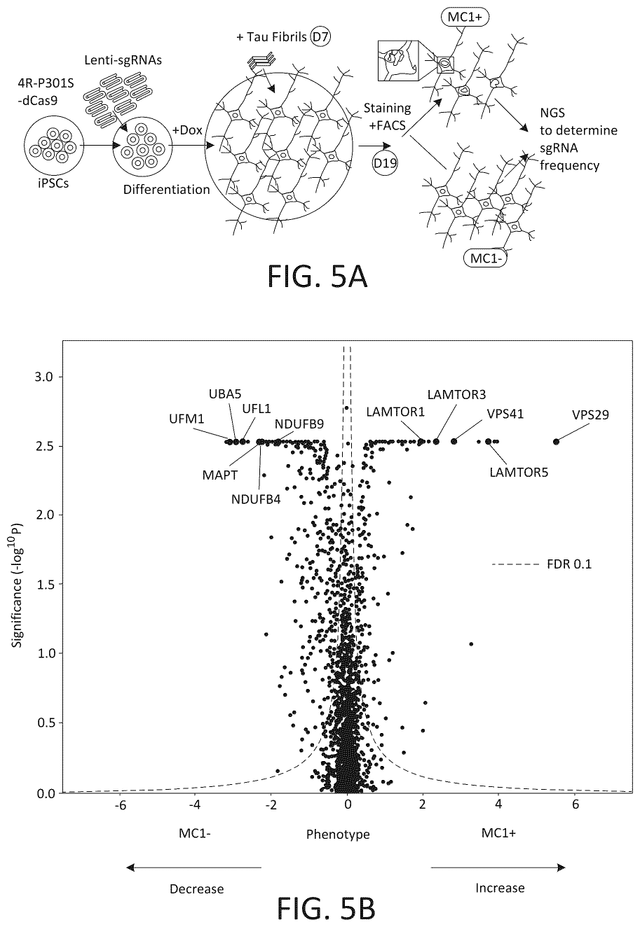

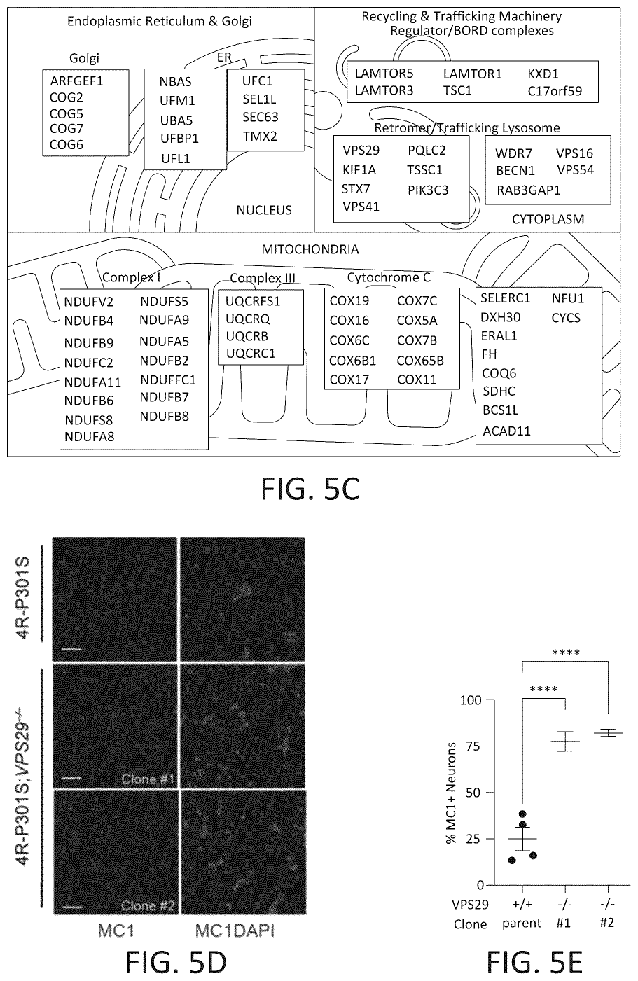

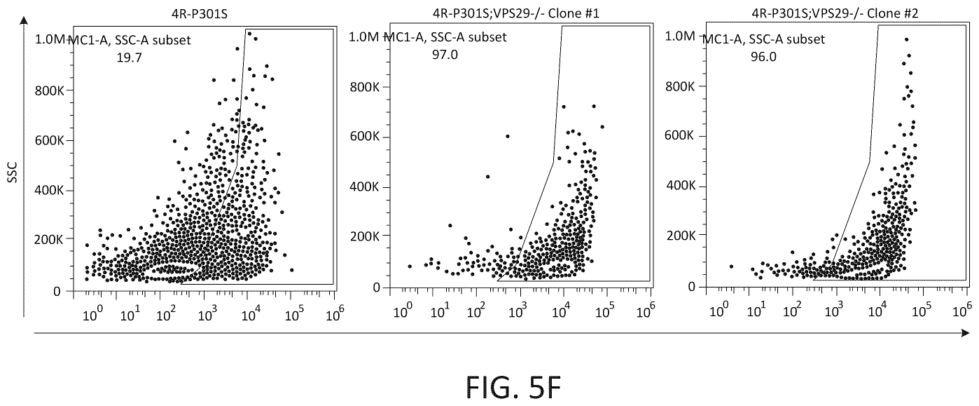

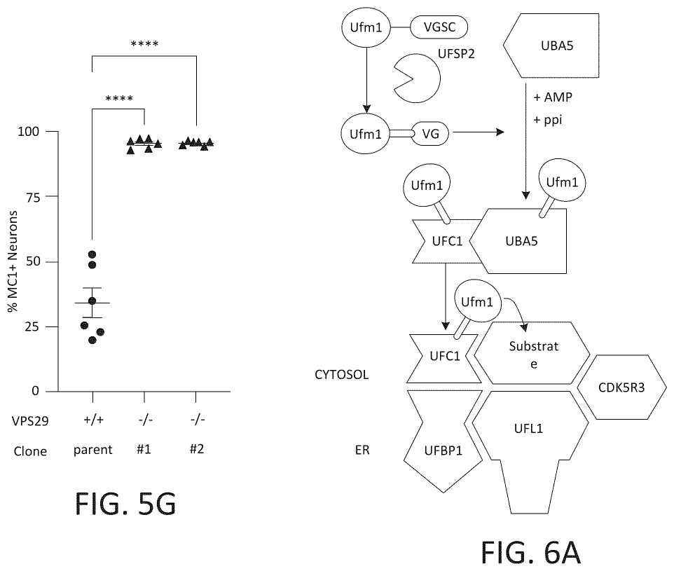

A custom lentiviral CRISPRi sgRNA library targeting genes involved in Tau pathobiology was used to transduce 4R-P301S-dCas9 iPSCs. This library consists of sgRNAs targeting 1,073 genes with five sgRNAs per gene and 250 non-targeting control (NTC) sgRNAs. After library transduction, iPSCs were differentiated into neurons, and K18-Tau was seeded at D7 (FIG. 5A). Neurons were collected at D19, fixed, and stained for MC1, and FACS sorted based on MC1 signal in the 488 nm channel into either MC1+ or MC1− bins (FIG. 5A). Frequencies of cells expressing each sgRNA were determined by next-generation sequencing to uncover genes for which sgRNAs showed significant changes in frequency, either an increase or a decrease in relation to Tau inclusion phenotype (FIG. 5A).

MAPT gRNAs were enriched in cells with reduced Tau inclusions as expected (FIG. 5B). Many of the other hits that reduced Tau inclusions are mitochondria genes, including many components in complex I (NDUFB9, NDUFV2, NDUFB4), complex II (UQCRQ, UQCRB), and cytochrome C (COX6C, COX7B, COX7C) (FIG. 5B, 5C). There was observed enrichment of genes involved in UFMylation, a post-translation modification analogous to ubiquitylation (UBA5, UFM1, UFBP1, UFL1, UFC1) (FIG. 5B, 5C), related to processes such as endoplasmic reticulum (ER)-associated protein degradation, ribosome-associated protein quality control at the ER and ER-phagy (Eldeeb et al., 2021; Liang et al., 2020). Gene set enrichment analyses revealed striking enrichment of distinct mitochondria and biosynthesis-related pathways (FIG. 12F).

The top genes whose knockdown increased Tau inclusions were strikingly enriched with those involved in multiple pathways of vesicular trafficking, as revealed with gene set enrichment analyses (FIG. 12E). Top hits include genes important for endolysosomal biogenesis and trafficking, such as LAMTOR complex (LAMTOR1, LAMTOR3, LAMTOR5), known as Late Endosomal/Lysosomal Adaptor and MAPK and mTOR Activator, as well as core subunits of HOPS (Homotypic fusion and Protein Sorting) complex, VPS16, and VPS41, which play a role in endosome-to-lysosome trafficking and fusion (FIG. 5C). Genes involved in Golgi trafficking, such as Conserved Oligomeric Golgi (COG) complex (COG2, COG5, COG6, COG7) (Sumya et al., 2023) are also strong modifiers of Tau aggregation. Inhibition of VPS54, a subunit of the GARP (Golgi-Associated Retrograde Protein) complex, also exacerbated Tau inclusion pathology (FIG. 5B, 5C). VPS54 is involved in the retrograde transport of vesicles from endosomes to the trans-Golgi network (TGN). Notably, VPS29, a subunit of the retromer complex, emerged as one of the strongest modifiers. The retromer complex is responsible for the retrograde transport of cargo from endosomes to the Golgi apparatus and the plasma membrane, and VPS29 contributes to the cargo recognition and stability of the retromer complex.

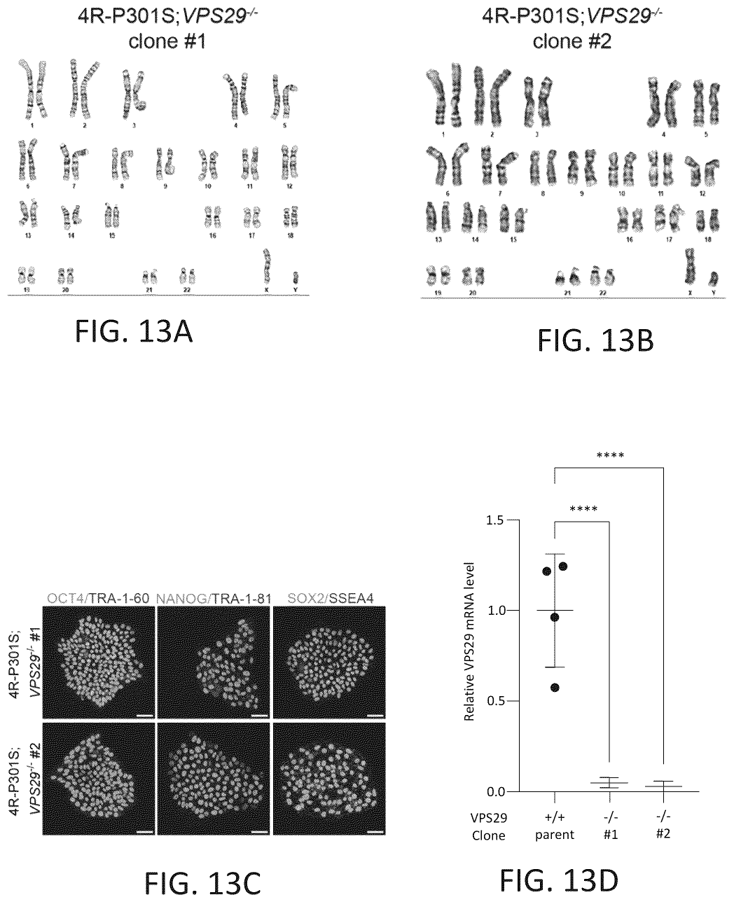

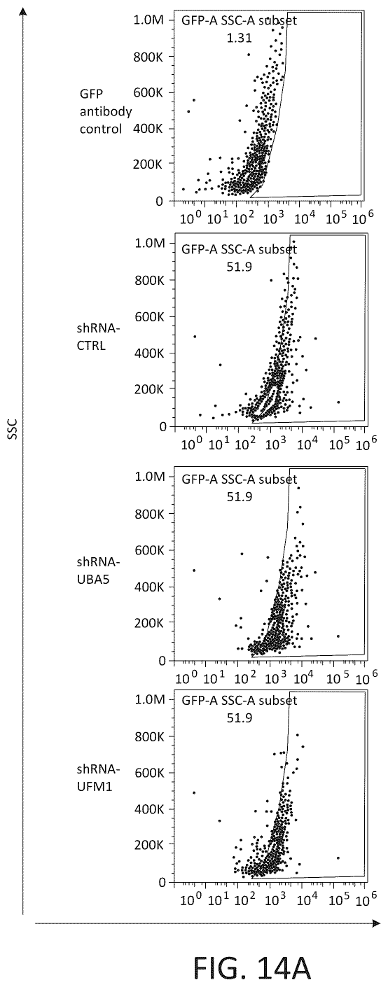

To validate the hits from the CRISPRi screen, VPS29 was deleted in 4R-P301S lines. Three independent clones of 4R-P301S;VPS29−/− lines were generated and confirmed with normal karyotypes and markers of pluripotency (FIG. 13A-C). Using qRT-PC, the deletion of VPS29 in clones #1 and #2 was confirmed (FIG. 13D). All three clones and the 4R-P301S parental line were differentiated into neurons and seeded with K18-tau fibrils. Three weeks after K18-Tau seeding, immunocytochemistry revealed a striking increase of MC1+ inclusions in all three clones of 4R-P301S;VPS29−/−, from 20% in the parental line to >80% in 4R-P301S;VPS29−/− neurons (FIG. 5D, 5E). To further confirm the effects of VPS29, FACS analyses were performed in two of the clones. Strikingly, more than 90% of 4R-P301 S;VPS29−/− neurons exhibit MC+ Tau inclusions, compared with 20-30% in the 4R-P301S neurons (FIG. 5F, 5G), establishing VPS29 as a predominant modifier of Tau propagation in the FTD human tauopathy model.

### UFMylation Cascade as a Novel Modifier of Tau Propagation

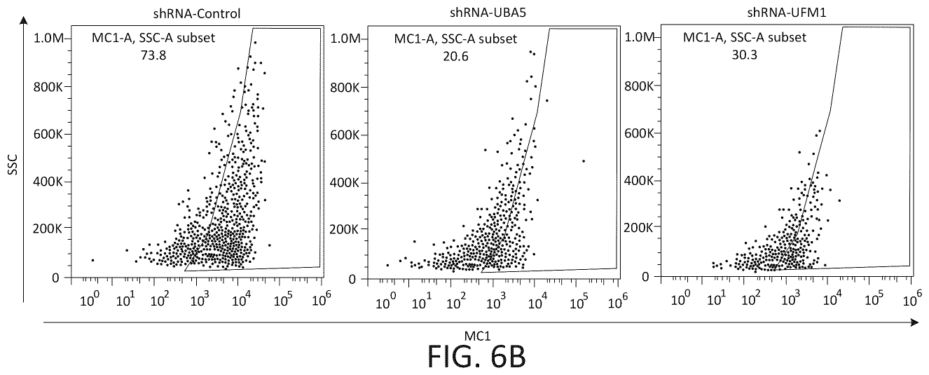

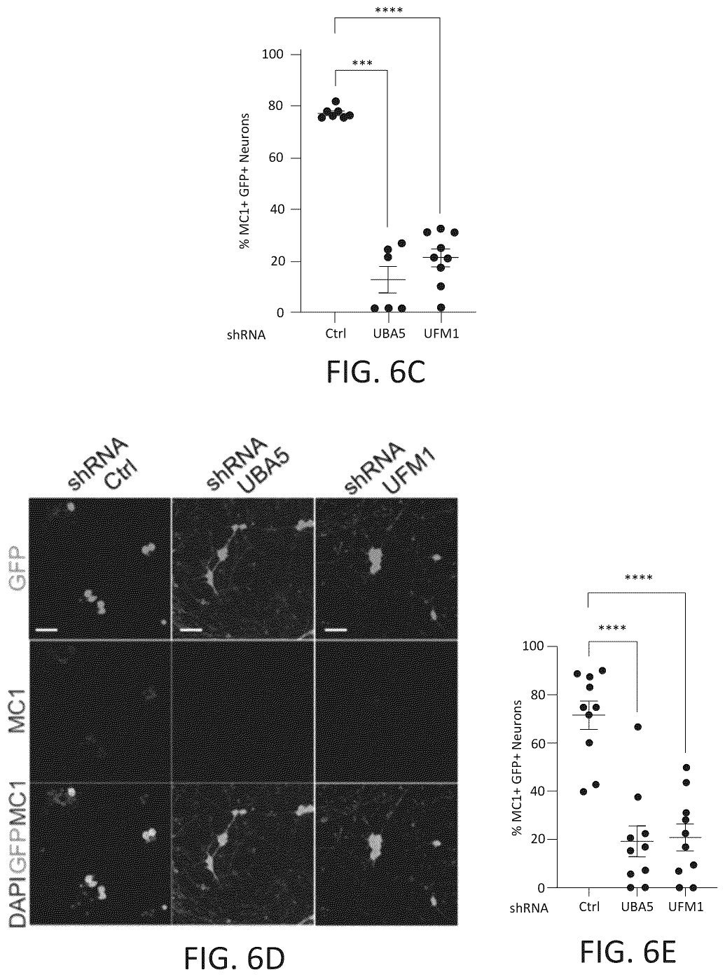

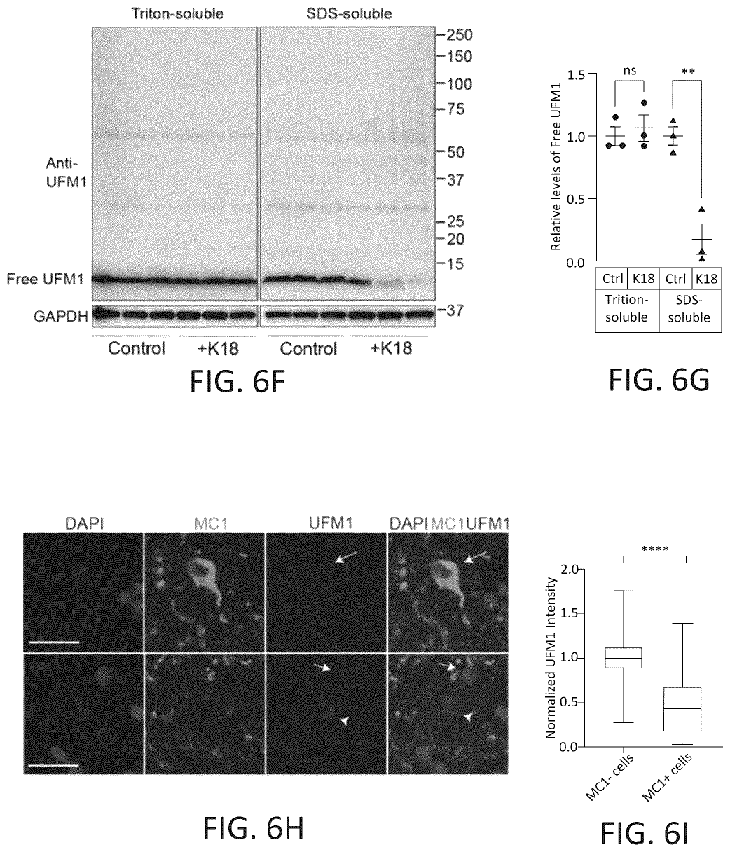

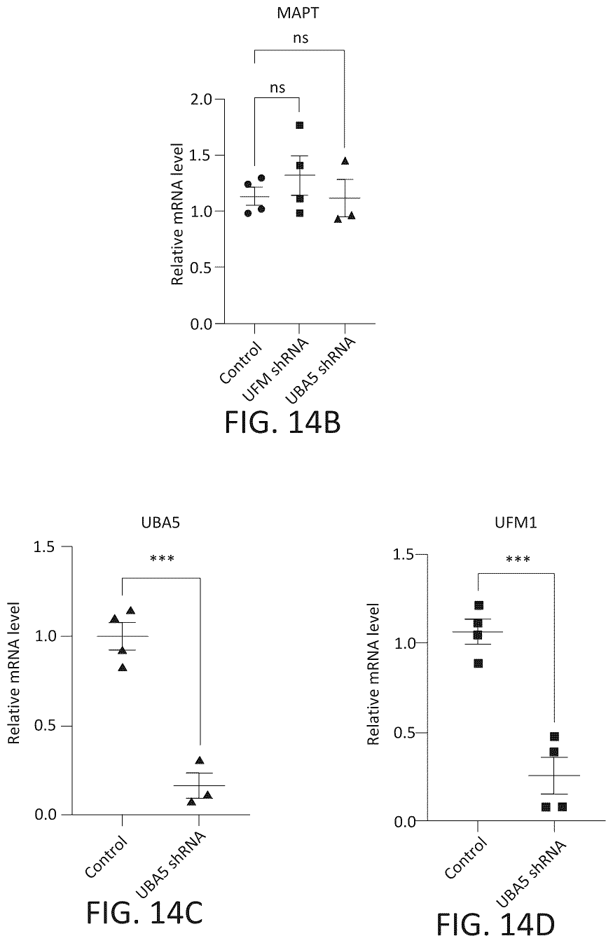

Next, the UFMylation cascade was selected to validate the sgRNA hits that reduce Tau inclusions. Among the top hits are five out of six components in the UFMylation pathway. UBA5 and UFL1 are E1-like enzymes that activate UFM1, UFC1 is an E2-like enzyme that catalyzes activated UFM1 to target proteins, and UFBP1 is involved in the recognition and binding of UFMylated substrates (FIG. 6A). Lentiviral constructs expressing UBA5, UFM1, or UFBP1 shRNAs under the U6 promoter and GFP under the SV40 promoter were generated for knockdown in human iPSC neurons. Using qRT-PCR, there was confirmation of an efficient knockdown of UBA5 and UFM1 (FIG. 14B, 14C), but knockdown of UFBP1 resulted in significant toxicity (data not shown). In flow cytometry analyses performed 21 days post-seeding, the MC1+ signal was measured in GFP+ neurons, so only those expressing the given shRNA were included (FIG. 14A). Seeding induced a robust MC1+ inclusions in neurons infected with control GFP-expressing lentivirus. However, MC1+/GFP+ neurons were markedly fewer with Lenti-shUBA5 or Lenti-shUFM1 than with Lenti-GFP controls (FIG. 6B, 6C). MC1+/GFP+ neurons were also quantified using immunocytochemistry. In agreement with flow cytometry analyses, infection with Lenti-shUBA5 or Lenti-shUFM1 resulting significantly fewer MC1+/GFP+ neurons than the Lenti-GFP controls (FIG. 6D, 6E). The inclusion-promoting effects of UFMylation are not due to its effect on MAPT mRNA since levels of MAPT mRNA were not affected by Lenti-shUBA5 nor Lenti-shUFM1.

The levels of UFMylation in K18-seeded and unseeded 4R-P301S neurons in Triton-soluble and Triton-insoluble (SDS soluble) lysates, a fraction enriched with high MW Tau species from 4R-P301S neurons with Tau inclusions, were quantified. Immunoblotting for an anti-UFM1 antibody preferentially recognizes free UFM1, revealed a strong reduction of free UFM1 only in the triton-insoluble fraction of seeded 4R-P301S neurons (FIG. 6F, 6G). To further explore the relevance of UFMylation in Tau pathology in AD, co-immunostaining of MC1 and UFM1 was performed. In AD brains, it was shown that MC1_tangle-bearing neurons exhibited lower levels of UFM1 immunoreactivity (FIG. 6H, 6I), consistent with lower levels of free UFM1 in lysates from neurons with Tau inclusions. These findings support that UFMylation promotes Tau pathology in AD.

UBA5 inhibitor Result

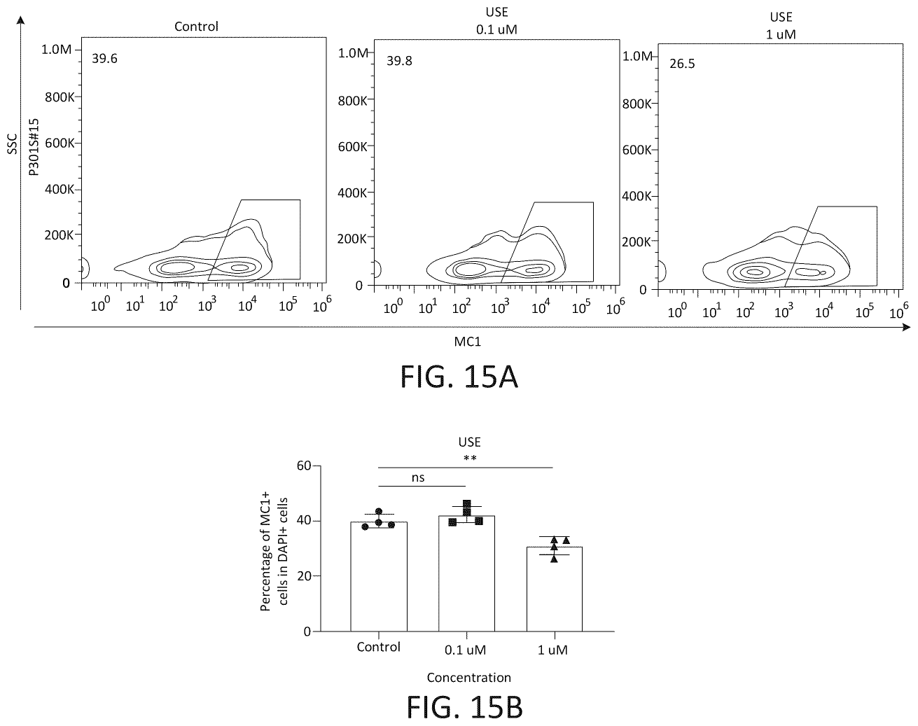

After confirmation that shRNA targeting UBA5 and UFM1 could rescue the tau inclusion induced by K18-seeding with both FACS and confocal-microscopy, the chemical library was examined and three UBA5 inhibitors were found. Next inhibitor rescue of tau inclusion induced by K18 treatment on the iPSC-derived neurons was determined as well. Scientists differentiated neurons in PDL-coated 12 well plates and treated cells on Day 5 with all three UBA5 inhibitors separately at various concentrations. On Day 7, 3.0 ug/mL K18 was administered to neurons and all samples were collected and fixed for FACS analysis on Day 21. Usenamine A at 1 uM successfully prevented tau inclusion induced by K18 seeding (MC1+/DAPI+ neurons) compared with control treatment group (FIGS. 15A and 15B). Usenamine A (USE) at lower concentration (0.1 uM) failed to prevent tau inclusion in neurons (FIGS. 15A and 15B) and exerted toxic effect on neurons at higher concentration (5 uM, 10 uM and 20 uM) (data not shown).

### Discussion

Here a robust 4R tauopathy model in iPSC-derived human neurons is reported, recapitulating seeding-induced propagation of Tau inclusions in human tauopathies with endogenous levels of Tau. This model extends the recent progress in modeling 4R tauopathy in human neurons (Capano et al., 2022; Manos et al., 2022) and provides a unique platform to investigate the cellular machinery underlying Tau propagation. Compared with other iPSC models with limited somatic inclusions (Manos et al., 2022), the pure 4R tauopathy model exhibits robust somatic 4R tau inclusions upon seeding of Tau fibrils in a time-dependent manner. Along with fibrillar Tau structures, striking subcellular alterations were observed in the soma of inclusion-bearing neurons, reminiscent of those in AD brains. Different from the direct programming of fibroblasts by viral-mediated transduction (Capano et al., 2022), the isogenic iPSC lines were engineered via CRISPR editing of splicing sites to express 4R-Tau or 4R-P301S-Tau, which allows the production of large quantities of neurons homogeneously and reproducibly, facilitating the application of CRISPRi/a screenings.

Given the close association of the progression of Tau aggregation with cognitive decline, there is an urgent need to model the propagation of tauopathy in human neurons. Despite obvious differences between the in vitro Tau inclusion model and tangle-bearing neurons in AD brains, significant transcriptional overlaps were observed, highlighting the pathophysiological relevance of the iPSC-derived tauopathy model. Moreover, TEM analyses of inclusion-bearing neurons revealed a striking accumulation of MLBs, consistent with the endolysosomal dysfunction implicated in tauopathy and other neurodegenerative conditions. However, unlike Tau inclusions in AD, PSP, and CBD, which consist of wild-type tau, 4R neurons lacking P301S mutation exhibited no Tau inclusions in the current experimental condition. There are many possible explanations, including the challenges of modeling a chronic process that takes decades in a culture that lasts weeks and the facts that P301S mutation likely accelerates the process greatly. Indeed, transcriptomic analyses revealed that P301S mutation by itself markedly downregulates genes involved in intracellular and membrane trafficking, which could render 4R-P301S neurons more vulnerable to Tau proteostasis imbalance triggered by seeding. Consistent with this notion, functional knockdown of the v-SNARE lysosome-associated protein, VAMP7, promoted Tau propagation. This platform enables systematic investigation on how strain selectivity, and/or selective vulnerability in cellular machinery, contributes to the heterogeneity of tauopathies.

The spread of Tau pathology often follows a predictable, trans-neuronal pattern, consistent with the role of neuronal activity in Tau spread (Franzmeier et al., 2022; Vogel et al., 2020). This tauopathy platform enabled the tracking of Tau pathology in live human neurons and uncovered intriguing crosstalk between neuronal activity and tau propagation. FTDP-17 patients carrying P301S mutation exhibit hyperexcitability (Garcia-Cabrero et al., 2013; Sperfeld et al., 1999). Other FTD mutations, such as V337M, also induce hyperexcitability in human neurons, suggesting a common pathogenic phenotype (Sohn et al., 2019). By chronically silencing neuronal activity using inhibitory DREADDs, Tau propagation was significantly ameliorated, providing direct evidence that hyperexcitability associated with P301S mutation could promote Tau propagation.

Despite the correlation between the spread of Tau aggregates with cognitive decline, the exact toxic species of Tau remain poorly understood (Gendron and Petrucelli, 2009; Guerrero-Munoz et al., 2015; Spires-Jones et al., 2009). It is challenging to measure neuronal function while simultaneously monitoring their Tau pathology, as it often requires antibody staining of fixed neurons. By Halo-Tagging 4R-P301 S tau, direct comparison of calcium influx of live neurons with or without somatic inclusions, provided the first direct evidence that Tau inclusions impair neuronal activity in human neurons. This observation challenges the view that Tau aggregates are not toxic per se and is consistent with the negative correlation of tauopathy measured by PET Tau tracer with neuronal activity measured with FDG PET (Zhang et al., 2021). This platform opens the door for a more detailed functional characterization of the effects of different Tau species in human neurons.

The cellular machinery underlying the spread of Tau aggregates remains poorly understood, partially due to a lack of an authentic robust Tau propagation model in human neurons. The current model enables systematic analyses of cellular machinery using functional genomics. In a CRISPRi screen of a curated set of >1000 genes implicated in Tau pathobiology, these scientists uncovered both known and unknown modifiers of Tau propagation. For example, one of the key steps of Tau propagation that remain poorly understood is how Tau seeds eventually escape the endocytic compartment and seed the aggregation of naive Tau in the cytoplasm. This CRISPRi screen revealed that the top genes whose knockdown increases inclusions are those involved in almost all aspects of vesicular trafficking and autophagy, including those involved in the endolysosomal biogenesis and trafficking, endosome-to-lysosome fusion, Golgi trafficking, retrograde transport of vesicles from endosomes to TGN, as well as a component of retromer complex required to transport cargos from endosome to TGN or plasma membrane. These findings suggest that any deficiency in vesicular trafficking could promote Tau propagation. Based on the CRISPR screen, it is proposed that the rate-limiting factor in propagation is the breaching of the organelle membranes and the escape of seeds from endocytic compartments, enabling the templating of naive Tau in the cytosol (Carosi et al., 2021).

As one of the core components of the retromer complex, VPS29 serves as a hub for vesicular trafficking (Banos-Mateos et al., 2019; Ye et al., 2020) and emerged as the top hit in this targeted screen. The two other components of the retromer complex, VPS35, whose mutation is linked with Parkinson's disease (Williams et al., 2017), and VPS26 are not targeted in the library. The profound modifying effects of VPS29 were validated by establishing two clones of 4R-P301S cells lacking VPS29. VPS29 deficiency markedly enhanced seeding-induced propagation by 2-3-fold. Retromer activity is diminished in the entorhinal cortex of late-onset AD patients (Small et al., 2005), consistent with data supporting the entorhinal cortex as one of the most vulnerable regions for Tau accumulation (Llamas-Rodriguez et al., 2022). In a Drosophila model that overexpresses human Tau, reduction of retromer activity induces a potent enhancement of Tau toxicity, including synapse loss, axon retraction, and survival via the production of a C-terminal-truncated isoform of hTau (Asadzadeh et al., 2022). Alterations in retromer activity induced by VPS35 with D620N mutation promote Tau aggregation in the forebrain of a mouse model of Parkinson's disease (Chen et al., 2019). In agreement with these findings, a deficiency in retromer complex activity exhibit increased levels of pathological Tau species in the cerebrospinal fluid (Simoes et al., 2020).

The proof-of-principle CRISPRi screen also uncovered novel modifiers of Tau propagation. While MAPT is among one of the top hits whose inhibition reduces Tau inclusions as expected, many of the top hits are mitochondrial genes, including many components in complex I, complex II, and cytochrome C. Interestingly, some of these genes overlap with the mitochondria-interacting proteins modified by FTD mutations in previous studies (Tracy et al., 2022). The mechanisms underlying the reduced propagation remain unknown. Since reduced neuronal activity ameliorates Tau propagation, lowering mitochondrial proteins might suppress neuronal activity, thus blocking the propagation.

Out of the top hits that promote Tau propagation are five out of the six genes in the UFMylation cascade, a process mediated by an enzyme cascade involving UBA5 (E1), UFC1 (E2), and an ER-localized trimeric ligase complex (E3) composed of UFL1, DDRGK1 (also known as UFBP1), and CDK5RAP3 (Gerakis et al., 2019, which is incorporated herein by reference for the UFMylation cascade and enzymes/proteins involved therein). Validating the CRISPRi screen, it was shown that inhibition of UFMylation by reducing UBA5 and UFM1 with shRNA markedly blocked Tau propagation. Using an antibody that preferentially binds to free UFM1, free UFM1 was reduced in inclusion-bearing neurons in the human 4R tauopathy model and tangle-bearing neurons in human AD brains. These findings provide the first evidence of involvement of UFMylation in tauopathy and that targeting UFMylation is beneficial to reduce propagation. UFMylation modulates several biological processes, and its deficiency leads to neurodevelopmental disorders (Nahorski et al., 2018). One of the principal targets of UFMylation is RPL26 (Walczak et al., 2019), whose UFMylation activates translocation-associated ribosomal quality control (Wang et al., 2023). Genetic inactivation of UFM1 or UFMylating enzymes causes the accumulation of translocation-stalled proteins at the ER and triggers ER stress. UFMylation is also strongly implicated in ER-phagy, a selective process that helps maintain ER homeostasis and cellular health by removing and recycling damaged, dysfunctional, or excessive ER components (Liang et al., 2020).

In summary, development and characterization of a 4R tauopathy human iPSC platform and identified known and novel genetic modifiers is provided herein. These novel 4R-P301S iPSC lines are a powerful platform to reveal the mechanisms underlying 4R tauopathies and identify new targets for drug development.

### Summary

Tauopathies are age-associated neurodegenerative diseases whose mechanistic underpinnings remain elusive, partially due to lack of appropriate human models. Current human induced pluripotent stem cell (hiPSC)-derived neurons express very low levels of 4-repeat (4R)-tau isoforms that are normally expressed in adult brain. Herein, new iPSC lines were engineered to express 4R-tau and 4R-tau carrying the P301S MAPT mutation when differentiated into neurons. 4R-P301S neurons display progressive Tau inclusions upon seeding with Tau fibrils and recapitulate features of tauopathy phenotypes, including shared transcriptomic signatures, autophagic body accumulation, and impaired neuronal activity. A CRISPRi screening of genes associated with Tau pathobiology identified over 500 genetic modifiers of Tau-seeding-induced Tau propagation, including retromer VPS29 and the UFMylation cascade as top modifiers. In AD brains, the UFMylation cascade is altered in neurofibrillary-tangle-bearing neurons. Inhibiting the UFMylation cascade suppressed seeding-induced Tau propagation. This model provides a powerful platform to identify novel therapeutic strategies for 4R tauopathy.

### BIBLIOGRAPHY

1. Allen, B., Ingram, E., Takao, M., Smith, M. J., Jakes, R., Virdee, K., Yoshida, H., Holzer, M., Craxton, M., Emson, P. C., et al. (2002). Abundant tau filaments and nonapoptotic neurodegeneration in transgenic mice expressing human P301 S tau protein. J Neurosci 22, 9340-9351. 10.1523/JNEUROSCI.22-21-09340.2002.
2. Anders, S., Pyl, P. T., and Huber, W. (2015). HTSeq—a Python framework to work with high-throughput sequencing data. Bioinformatics 31, 166-169. 10.1093/bioinformatics/btu638.
3. Arantes, R. M., and Andrews, N. W. (2006). A role for synaptotagmin VII-regulated exocytosis of lysosomes in neurite outgrowth from primary sympathetic neurons. J Neurosci 26, 4630-4637. 10.1523/JNEUROSCI.0009-06.2006.
4. Asadzadeh, J., Ruchti, E., Jiao, W., Limoni, G., MacLachlan, C., Small, S. A., Knott, G., Santa-Maria, I., and McCabe, B. D. (2022). Retromer deficiency in Tauopathy models enhances the truncation and toxicity of Tau. Nat Commun 13, 5049. 10.1038/s41467-022-32683-5.
5. Banos-Mateos, S., Rojas, A. L., and Hierro, A. (2019). VPS29, a tweak tool of endosomal recycling. Curr Opin Cell Biol 59, 81-87. 10.1016fj.ceb.2019.03.010.
6. Bejanin, A., Schonhaut, D. R., La Joie, R., Kramer, J. H., Baker, S. L., Sosa, N., Ayakta, N., Cantwell, A., Janabi, M., Lauriola, M., et al. (2017). Tau pathology and neurodegeneration contribute to cognitive impairment in Alzheimer's disease. Brain 140, 3286-3300. 10.1093/brain/awx243.
7. Berriman, J., Serpell, L. C., Oberg, K. A., Fink, A. L., Goedert, M., and Crowther, R. A. (2003). Tau filaments from human brain and from in vitro assembly of recombinant protein show cross-beta structure. Proc Natl Acad Sci USA 100, 9034-9038. 10.1073/pnas.1530287100.
8. Bruno, M.; Trucchi, B.; Burlando, B., Ranzato, E.; Martinotti, S., Akkol, E. K.; Suntar, I.; Keleq, H., Verotta, L. (+)-Usnic acid enamines with remarkable cicatrizing properties. Bioorganic & Medicinal Chemistry 2013, 21 (7), 1834-1843. DOI: https://doi.org/10.1016/j.bmc.2013.01.045.
9. Capano, L. S., Sato, C., Ficulle, E., Yu, A., Horie, K., Kwon, J. S., Burbach, K. F., Barthelemy, N. R., Fox, S. G., Karch, C. M., et al. (2022). Recapitulation of endogenous 4R tau expression and formation of insoluble tau in directly reprogrammed human neurons. Cell Stem Cell 29, 918-932 e918. 10.1016/j.stem.2022.04.018.
10. Carosi, J. M., Denton, D., Kumar, S., and Sargeant, T. J. (2021). Retromer dysfunction at the nexus of tauopathies. Cell Death Differ 28, 884-899. 10.1038/s41418-020-00727-2.
11. Chen, X., Kordich, J. K., Williams, E. T., Levine, N., Cole-Strauss, A., Marshall, L., Labrie, V., Ma, J., Lipton, J. W., and Moore, D. J. (2019). Parkinson's disease-linked D620N VPS35 knockin mice manifest tau neuropathology and dopaminergic neurodegeneration. Proc Natl Acad Sci USA 116, 5765-5774. 10.1073/pnas.1814909116.
12. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
13. Eldeeb, M. A., Zorca, C. E., Ragheb, M. A., Rashidi, F. B., and Salah E1-Din, D. S. (2021). Fine-tuning ER-phagy by post-translational modifications. Bioessays 43, e2000212. 10.1002/bies.202000212.
14. Espinoza, M., de Silva, R., Dickson, D. W., and Davies, P. (2008). Differential incorporation of tau isoforms in Alzheimer's disease. J Alzheimers Dis 14, 1-16. 10.3233/jad-2008-14101.
15. Fitzpatrick, A. W. P., Falcon, B., He, S., Murzin, A. G., Murshudov, G., Garringer, H. J., Crowther, R. A., Ghetti, B., Goedert, M., and Scheres, S. H. W. (2017). Cryo-EM structures of tau filaments from Alzheimer's disease. Nature 547, 185-190. 10.1038/nature23002.
16. Franzmeier, N., Brendel, M., Beyer, L., Slemann, L., Kovacs, G. G., Arzberger, T., Kurz, C., Respondek, G., Lukic, M. J., Biel, D., et al. (2022). Tau deposition patterns are associated with functional connectivity in primary tauopathies. Nat Commun 13, 1362. 10.1038/s41467-022-28896-3.
17. Garcia-Cabrero, A. M., Guerrero-Lopez, R., Giraldez, B. G., Llorens-Martin, M., Avila, J., Serratosa, J. M., and Sanchez, M. P. (2013). Hyperexcitability and epileptic seizures in a model of frontotemporal dementia. Neurobiol Dis 58, 200-208. 10.1016fj.nbd.2013.06.005.
18. Gendron, T. F., and Petrucelli, L. (2009). The role of tau in neurodegeneration. Mol Neurodegener 4, 13. 10.1186/1750-1326-4-13.
19. Gerakis, Y, Quintero, M., Li, H., and Hetz, C. (2019). The UFMylation System in Proteostasis and Beyond. Trends Cell Biol 29, 974-986. 10.1016/j.tcb.2019.09.005.
20. Gilbert, L. A., Horibeck, M. A., Adamson, B., Villalta, J. E., Chen, Y, Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661. 10.1016fj.cell.2014.09.029.
21. Goedert, M., Spillantini, M. G., Jakes, R., Rutherford, D., and Crowther, R. A. (1989). Multiple isoforms of human microtubule-associated protein tau: sequences and localization in neurofibrillary tangles of Alzheimer's disease. Neuron 3, 519-526. 10.1016/0896-6273(89)90210-9.
22. Gotz, J., Halliday, G., and Nisbet, R. M. (2019). Molecular Pathogenesis of the Tauopathies. Annu Rev Pathol 14, 239-261. 10.1146/annurev-pathmechdis-012418-012936.
23. Grimm, J. B., English, B. P, Chen, J., Slaughter, J. P., Zhang, Z., Revyakin, A., Patel, R., Macklin, J. J., Normanno, D., Singer, R. H., et al. (2015). A general method to improve fluorophores for live-cell and single-molecule microscopy. Nat Methods 12, 244-250, 243 p following 250. 10.1038/nmeth.3256.
24. Guerrero-Munoz, M. J., Gerson, J., and Castillo-Carranza, D. L. (2015). Tau Oligomers: The Toxic Player at Synapses in Alzheimer's Disease. Front Cell Neurosci 9, 464. 10.3389/fncel.2015.00464.
25. Gustke, N., Trinczek, B., Biernat, J., Mandelkow, E. M., and Mandelkow, E. (1994). Domains of tau Protein and Interactions with Microtubules. Biochemistry 33, 9511-9522. 10.1021/bi00198a017.
26. Hariri, M., Millane, G., Guimond, M P., Guay, G., Dennis, J. W., and Nabi, I. R. (2000). Biogenesis of multilamellar bodies via autophagy. Mol Biol Cell 11, 255-268. 10.1091/mbc.11.1.255.
27. Horlbeck, M. A., Gilbert, L. A., Villalta, J. E., Adamson, B., Pak, R. A., Chen, Y., Fields, A. P., Park, C. Y., Corn, J. E., Kampmann, M., and Weissman, J. S. (2016). Compact and highly active next-generation libraries for CRISPR-mediated gene repression and activation. Elife 5. 10.7554/eLife.19760.
28. Hutton, M., Lendon, C. L., Rizzu, P, Baker, M., Froelich, S., Houlden, H., Pickering-Brown, S., Chakraverty, S., Isaacs, A., Grover, A., et al. (1998). Association of missense and 5′-splice-site mutations in tau with the inherited dementia FTDP-17. Nature 393, 702-705. 10.1038/31508.
29. Ito, S., and Karnovsky, M. J. (1968). Formaldehyde-Glutaraldehyde Fixatives Containing Trinitro Compounds Journal of Cell Biology 39, 168A-169A.
30. Jerber, J., Seaton, D. D., Cuomo, A. S. E., Kumasaka, N., Haldane, J., Steer, J., Patel, M., Pearce, D., Andersson, M., Bonder, M. J., et al. (2021). Population-scale single-cell RNA-seq profiling across dopaminergic neuron differentiation. Nat Genet 53, 304-312. 10.1038/s41588-021-00801-6.
31. Jia, H., Rochefort, N. L., Chen, X., and Konnerth, A. (2011). In vivo two-photon imaging of sensory-evoked dendritic calcium signals in cortical neurons. Nat Protoc 6, 28-35. 10.1038/nprot.2010.169.
32. Jicha, G. A., Bowser, R., Kazam, I. G., and Davies, P. (1997). Alz-50 and MC-1, a new monoclonal antibody raised to paired helical filaments, recognize conformational epitopes on recombinant tau. J Neurosci Res 48, 128-132. 10.1002/(sici)1097-4547(19970415)48:2<128::aid-jnr5>3.0.co;2-e.
33. Kampmann, M., Bassik, M. C., and Weissman, J. S. (2014). Functional genomics platform for pooled screening and generation of mammalian genetic interaction maps. Nat Protoc 9, 1825-1847. 10.1038/nprot.2014.103.
34. Karch, C. M., Kao, A. W., Karydas, A., Onanuga, K., Martinez, R., Argouarch, A., Wang, C., Huang, C., Sohn, P. D., Bowles, K. R., et al. (2019). A Comprehensive Resource for Induced Pluripotent Stem Cells from Patients with Primary Tauopathies. Stem Cell Reports 13, 939-955. 10.1016/j.stemcr.2019.09.006.
35. Liang, J. R., Lingeman, E., Luong, T., Ahmed, S., Muhar, M., Nguyen, T., Olzmann, J. A., and Corn, J. E. (2020). A Genome-wide ER-phagy Screen Highlights Key Roles of Mitochondrial Metabolism and ER-Resident UFMylation. Cell 180, 1160-1177 e1120. 10.1016/j.cell.2020.02.017.
36. Liberzon, A., Subramanian, A., Pinchback, R., Thorvaldsdottir, H., Tamayo, P, and Mesirov, J. P. (2011). Molecular signatures database (MSigDB) 3.0. Bioinformatics 27, 1739-1740. 10.1093/bioinformatics/btr260.
37. Llamas-Rodriguez, J., Oltmer, J., Greve, D. N., Williams, E., Slepneva, N., Wang, R., Champion, S., Lang-Orsini, M., Fischl, B., Frosch, M. P., et al. (2022). Entorhinal Subfield Vulnerability to Neurofibrillary Tangles in Aging and the Preclinical Stage of Alzheimer's Disease. J Alzheimers Dis 87, 1379-1399. 10.3233/JAD-215567.
38. Love, M. I., Huber, W., and Anders, S. (2014). Moderated estimation of fold change and dispersion for RNA-seq data with DESeq2. Genome Biol 15, 550. 10.1186/s13059-014-0550-8.
39. Manos, J. D., Preiss, C. N., Venkat, N., Tamm, J., Reinhardt, P., Kwon, T., Wu, J., Winter, A. D., Jahn, T. R., Yanamandra, K., et al. (2022). Uncovering specificity of endogenous TAU aggregation in a human iPSC-neuron TAU seeding model. iScience 25, 103658. 10.1016/j.isci.2021.103658.
40. Martin, M. (2011). Cutadapt Removes Adapter Sequences From High-Throughput Sequencing Reads. EMBnet.journal 17, 10-12. doi.org/10.14806/ej.17.1.200.
41. Mirra, S. S., Murrell, J. R., Gearing, M., Spillantini, M. G., Goedert, M., Crowther, R. A., Levey, A. I., Jones, R., Green, J., Shoffner, J. M., et al. (1999). Tau pathology in a family with dementia and a P301L mutation in tau. J Neuropathol Exp Neurol 58, 335-345. 10.1097/00005072-199904000-00004.
42. Miyaoka, Y, Chan, A. H., Judge, L. M., Yoo, J., Huang, M., Nguyen, T. D., Lizarraga, P. P., So, P. L., and Conklin, B. R. (2014). Isolation of single-base genome-edited human iPS cells without antibiotic selection. Nat Methods 11, 291-293. 10.1038/nmeth.2840.
43. Mok, S. A., Condello, C., Freilich, R., Gillies, A., Arhar, T., Oroz, J., Kadavath, H., Julien, O., Assimon, VA., Rauch, J. N., et al. (2018). Mapping interactions with the chaperone network reveals factors that protect against tau aggregation. Nat Struct Mol Biol 25, 384-393. 10.1038/s41594-018-0057-1.
44. Nahorski, M. S., Maddirevula, S., Ishimura, R., Alsahli, S., Brady, A. F., Begemann, A., Mizushima, T., Guzman-Vega, F. J., Obata, M., Ichimura, Y., et al. (2018). Biallelic UFM1 and UFC1 mutations expand the essential role of ufmylation in brain development. Brain 141, 1934-1945. 10.1093/brain/awy135.
45. Ossenkoppele, R., Pichet Binette, A., Groot, C., Smith, R., Strandberg, O., Palmqvist, S., Stomrud, E., Tideman, P., Ohlsson, T., Jogi, J., et al. (2022). Amyloid and tau PET-positive cognitively unimpaired individuals are at high risk for future cognitive decline. Nat Med 28, 2381-2387. 10.1038/s41591-022-02049-x.
46. Otero-Garcia, M., Mahajani, S. U., Wakhloo, D., Tang, W., Xue, Y. Q., Morabito, S., Pan, J., Oberhauser, J., Madira, A. E., Shakouri, T., et al. (2022). Molecular signatures underlying neurofibrillary tangle susceptibility in Alzheimers disease. Neuron 110, 2929-2948 e2928. 10.1016/j.neuron.2022.06.021.
47. Paonessa, F., Evans, L. D., Solanki, R., Larrieu, D., Wray, S., Hardy, J., Jackson, S. P., and Livesey, F. J. (2019). Microtubules Deform the Nuclear Membrane and Disrupt Nucleocytoplasmic Transport in Tau-Mediated Frontotemporal Dementia. Cell Rep 26, 582-593 e585. 10.1016/j.celrep.2018.12.085.
48. Pooler, A. M., Phillips, E. C., Lau, D. H., Noble, W., and Hanger, D. P. (2013). Physiological release of endogenous tau is stimulated by neuronal activity. EMBO Rep 14, 389-394. 10.1038/embor.2013.15.
49. Shen, L. S. I. (2022). GeneOverlap: Test and visualize gene overlaps. R package version 1.34.0. http://shenlab-sinai.github.io/shenlab-sinai/.
50. Shi, Y., Zhang, W., Yang, Y., Murzin, A. G., Falcon, B., Kotecha, A., van Beers, M., Tarutani, A., Kametani, F., Garringer, H. J., et al. (2021). Structure-based classification of tauopathies. Nature 598, 359-363. 10.1038/s41586-021-03911-7.
51. Simoes, S., Neufeld, J. L., Triana-Baltzer, G., Moughadam, S., Chen, E. I., Kothiya, M., Qureshi, Y. H., Patel, V., Honig, L. S., Kolb, H., and Small, S. A. (2020). Tau and other proteins found in Alzheimer's disease spinal fluid are linked to retromer-mediated endosomal traffic in mice and humans. Sci Transl Med 12. 10.1126/scitranslmed.aba6334.
52. Small, S. A., Kent, K., Pierce, A., Leung, C., Kang, M. S., Okada, H., Honig, L., Vonsattel, J. P., and Kim, T. W. (2005). Model-guided microarray implicates the retromer complex in Alzheimer's disease. Ann Neurol 58, 909-919. 10.1002/ana.20667.
53. Sohn, PD., Huang, C. T., Yan, R., Fan, L., Tracy, T. E., Camargo, C. M., Montgomery, K. M., Arhar, T., Mok, S. A., Freilich, R., et al. (2019). Pathogenic Tau Impairs Axon Initial Segment Plasticity and Excitability Homeostasis. Neuron 104, 458-470 e455. 10.1016fj.neuron.2019.08.008.
54. Sperfeld, A. D., Collatz, M. B., Baier, H., Palmbach, M., Storch, A., Schwarz, J., Tatsch, K., Reske, S., Joosse, M., Heutink, P., and Ludolph, A. C. (1999). FTDP-17: an early-onset phenotype with parkinsonism and epileptic seizures caused by a novel mutation. Ann Neurol 46, 708-715. 10.1002/1531-8249(199911)46:5<708::aid-ana5>3.0.co;2-k.
55. Spillantini, M. G., Murrell, J. R., Goedert, M., Farlow, M. R., Klug, A., and Ghetti, B. (1998). Mutation in the tau gene in familial multiple system tauopathy with presenile dementia. Proc Natl Acad Sci USA 95, 7737-7741. 10.1073/pnas.95.13.7737.
56. Spires-Jones, T. L., Stoothoff, W. H., de Calignon, A., Jones, P. B., and Hyman, B. T. (2009). Tau pathophysiology in neurodegeneration: a tangled issue. Trends Neurosci 32, 150-159. 10.1016/j.tins.2008.11.007.
57. Sposito, T., Preza, E., Mahoney, C. J., Seto-Salvia, N., Ryan, N. S., Morris, H. R., Arber, C., Devine, M. J., Houlden, H., Warner, T. T., et al. (2015). Developmental regulation of tau splicing is disrupted in stem cell-derived neurons from frontotemporal dementia patients with the 10+16 splice-site mutation in MAPT. Hum Mol Genet 24, 5260-5269. 10.1093/hmg/ddv246.
58. Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550. 10.1073/pnas.0506580102.
59. Sumya, F. T., Pokrovskaya, I.D., D'Souza, Z., and Lupashin, V. V. (2023). Acute COG complex inactivation unveiled its immediate impact on Golgi and illuminated the nature of intra-Golgi recycling vesicles. Traffic 24, 52-75. 10.1111/tra.12876.
60. Sun, Z., and Sudhof, T. C. (2021). A simple Ca2+-imaging approach to neural network analyses in cultured neurons. Journal of Neuroscience Methods 349, 109041. https://doi.org/10.1016/j.jneumeth.2020.109041.
61. Tian, R., Gachechiladze, M. A., Ludwig, C. H., Laurie, M. T., Hong, J. Y., Nathaniel, D., Prabhu, AV, Fernandopulle, M. S., Patel, R., Abshari, M., et al. (2019). CRISPR Interference-Based Platform for Multimodal Genetic Screens in Human iPSC-Derived Neurons. Neuron 104, 239-255 e212. 10.1016/j.neuron.2019.07.014.
62. Tippani, M., Pattie, E. A., Davis, B. A., Nguyen, C. V., Wang, Y., Sripathy, S. R., Maher, B. J., Martinowich, K., Jaffe, A. E., and Page, S. C. (2022). CaPTure: Calcium PeakToolbox for analysis of in vitro calcium imaging data. BMC Neurosci 23, 71. 10.1186/s12868-022-00751-7.
63. Tracy, T. E., Madero-Perez, J., Swaney, D. L., Chang, T. S., Moritz, M., Konrad, C., Ward, M. E., Stevenson, E., Huttenhain, R., Kauwe, G., et al. (2022). Tau interactome maps synaptic and mitochondrial processes associated with neurodegeneration. Cell 185, 712-728 e714. 10.1016/j.cell.2021.12.041.
64. Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat Biotechnol 28, 511-515. 10.1038/nbt.1621.
65. Venable, J. H., and Coggeshall, R. (1965). A Simplified Lead Citrate Stain for Use in Electron Microscopy. J Cell Biol 25, 407-408. 10.1083/jcb.25.2.407.
66. Verheyen, A., Diels, A., Dijkmans, J., Oyelami, T., Meneghello, G., Mertens, L., Versweyveld, S., Borgers, M., Buist, A., Peeters, P., and Cik, M. (2015). Using Human iPSC-Derived Neurons to Model TAU Aggregation. PLoS One 10, e0146127. 10.1371/journal.pone.0146127.
67. Vogel, J. W., Iturria-Medina, Y., Strandberg, O. T., Smith, R., Levitis, E., Evans, A. C., Hansson, O., Alzheimer's Disease Neuroimaging, I., and Swedish BioFinder, S. (2020). Spread of pathological tau proteins through communicating neurons in human Alzheimer's disease. Nat Commun 11, 2612. 10.1038/s41467-020-15701-2.
68. Walczak, C. P., Leto, D. E., Zhang, L., Riepe, C., Muller, R. Y., DaRosa, P. A., Ingolia, N. T., Elias, J. E., and Kopito, R. R. (2019). Ribosomal protein RPL26 is the principal target of UFMylation. Proc Natl Acad Sci USA 116, 1299-1308. 10.1073/pnas.1816202116.
69. Wang, C., Ward, M. E., Chen, R., Liu, K., Tracy, T. E., Chen, X., Xie, M., Sohn, P. D., Ludwig, C., Meyer-Franke, A., et al. (2017). Scalable Production of iPSC-Derived Human Neurons to Identify Tau-Lowering Compounds by High-Content Screening. Stem Cell Reports 9, 1221-1233. 10.1016/j.stemcr.2017.08.019.
70. Wang, L., Xu, Y, Yun, S., Yuan, Q., Satpute-Krishnan, P., and Ye, Y. (2023). SAYSD1 senses UFMylated ribosome to safeguard co-translational protein translocation at the endoplasmic reticulum. Cell Rep 42, 112028. 10.1016/j.celrep.2023.112028.
71. Williams, E. T., Chen, X., and Moore, D. J. (2017). VPS35, the Retromer Complex and Parkinson's Disease. J Parkinsons Dis 7, 219-233. 10.3233/JPD-161020.
72. Wu, J. W., Hussaini, S. A., Bastille, I. M., Rodriguez, G. A., Mrejeru, A., Rilett, K., Sanders, D. W., Cook, C., Fu, H., Boonen, R. A., et al. (2016). Neuronal activity enhances tau propagation and tau pathology in vivo. Nat Neurosci 19, 1085-1092. 10.1038/nn.4328.
73. Xie, Y. X., Naseri, N. N., Fels, J., Kharel, P., Na, Y, Lane, D., Burre, J., and Sharma, M. (2022). Lysosomal exocytosis releases pathogenic alpha-synuclein species from neurons in synucleinopathy models. Nat Commun 13, 4918. 10.1038/s41467-022-32625-1.
74. Yamada, K., Holth, J. K., Liao, F., Stewart, F. R., Mahan, T. E., Jiang, H., Cirrito, J. R., Patel, T. K., Hochgrafe, K., Mandelkow, E. M., and Holtzman, D. M. (2014). Neuronal activity regulates extracellular tau in vivo. J Exp Med 211, 387-393. 10.1084/jem.20131685.
75. Yasuda, M., Nakamura, Y, Kawamata, T., Kaneyuki, H., Maeda, K., and Komure, O. (2005). Phenotypic heterogeneity within a new family with the MAPT p301s mutation. Ann Neurol 58, 920-928. 10.1002/ana.20668.
76. Ye, H., Ojelade, S. A., Li-Kroeger, D., Zuo, Z., Wang, L., Li, Y., Gu, J. Y., Tepass, U., Rodal, A. A., Bellen, H. J., and Shulman, J. M. (2020). Retromer subunit, VPS29, regulates synaptic transmission and is required for endolysosomal function in the aging brain. Elife 9. 10.7554/eLife.51977.
77. Zhang, K., Mizuma, H., Zhang, X., Takahashi, K., Jin, C., Song, F., Gao, Y., Kanayama, Y., Wu, Y., Li, Y., et al. (2021). PET imaging of neural activity, beta-amyloid, and tau in normal brain aging. Eur J Nucl Med Mol Imaging 48, 3859-3871. 10.1007/s00259-021-05230-5.
78. Zhang, Y., Rozsa, M., Liang, Y., Bushey, D., Wei, Z., Zheng, J., Reep, D., Broussard, G. J., Tsang, A., Tsegaye, G., et al. (2023). Fast and sensitive GCaMP calcium indicators for imaging neural populations. Nature. 10.1038/s41586-023-05828-9.
79. Zhu, H., and Roth, B. L. (2014). Silencing synapses with DREADDs. Neuron 82, 723-725. 10. 1016/j.neuron.2014.05.002.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications. In the event that the definition of a term incorporated by reference conflicts with a term defined herein, this specification shall control.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed is:

1. An induced pluripotent stem cell (iPSC) stably expressing 4R-Tau.

2. The iPSC of claim 1, wherein the iPSC is prepared from a fibroblast cell.

3. The iPSC of claim 1, wherein the iPSC is a human cell.

4. The iPSC of claim 1, wherein the iPSC comprising one or more mutations in the 5′ and or 3′ end of exon 10 of the microtubule associated protein tau (MAPT) gene.

5. The iPSC of claim 1, further comprising inducible expression of Neurogenin-2 transcription factor (NGN2).

6. The iPSC of claim 1, further comprising a nucleic acid mutation in one or both alleles of microtubule associated protein tau (MAPT) so at to result in a mutation at amino acid 301 of MAPT protein.

7. The iPSC of claim 6, wherein the amino acid mutation is P301S.

8. The iPSC of claim 7, wherein the P301S mutation occurs in SEQ ID NO: 3 or a polypeptide having 90% identity thereto.

9. The iPSC of claim 6 further comprising a Cas enzyme.

10. The iPSC of claim 9, wherein the Cas enzyme is Cas9.

11. A composition comprising iPSCs of claim 1.

12. A method to express 4R-tau, comprising differentiating the iPSC of claim 6 to a neuronal cell.

13. The method of claim 12, wherein the iPSC is contacted with one or more of brain-derived neurotrophic (BDNF), neurotrophin-3 (NTS), ROCK inhibitor or doxycycline.

14. A method to generate tau bundles/inclusions comprising contacting said neuronal cell of claim 12 with Tau fibrils.

15. The method of claim 15, wherein the Tau fibrils have one or more mutations compared to wild type.

16. A method to inhibit formation of Tau bundles/inclusions comprising contacting a neuronal cell with an inhibitor of an UFMylation pathway protein.

17. The method of claim 16, wherein the UFMylation pathway protein is one or more of UBA5 (E1), UFC1 (E2), UFL1, DDRGK1 and/or CDK5RAP3.

18. The method of claim 16, wherein the inhibitor a small molecule.

19. The method of claim 18, wherein the small molecule inhibitor is Usenamine A.

20. The method of claim 16, where in the inhibitor is an inhibitory nucleic acid sequence.

21. The method of claim 20, wherein the nucleic sequence is an shRNA, a small interfering RNA, a ribozyme or an antisense nucleic acid molecule.

22. The method of claim 21, wherein the nucleic sequence knocks down UBA5, UFM1, UFBP1 or a combination thereof.

22. The method of claim 21, wherein the shRNA has the sequence of any one of SEQ ID NOs: 4 to 11.

23. A method to treat a tauopathy comprising administering to a subject in need thereof an inhibitor of an UFMylation pathway protein.

24. The method of claim 23, wherein the tauopathy is Alzheimer's disease (AD), frontotemporal lobar degeneration with Tau pathology (FTLD-Tau), corticobasal degeneration (CBD), progressive supranuclear palsy (PSP), argyrophilic grain disease (AGD), globular glial tauopathy, chronic traumatic encephalopathy (CTE) or Pick's disease (PiD).

25. A method to screen for compounds that inhibit formation of Tau bundles/inclusions comprising
contacting said neuronal cell of claim 12 with Tau fibrils and a test agent;
detecting the presence or absence of Tau bundles/inclusions,
wherein the absence of Tau bundles/inclusions correlates with the test agent being a compound that inhibits formation of Tau bundles/inclusions.
